SEQUENCE LISTING (11 sequences)

SEQ ID NO: 1 - protein, 6 aa, Artificial Sequence
  Synthetic Peptide
SLIGRL

SEQ ID NO: 2 - DNA, 24 bp, Artificial Sequence
  Synthetic primer
ggggagccaaaagggtcatcatct

SEQ ID NO: 3 - DNA, 24 bp, Artificial Sequence
  Synthetic primer
gacgcctgcttcaccaccttcttg

SEQ ID NO: 4 - DNA, 24 bp, Artificial Sequence
  Synthetic primer
cggaggtcatcgccagcatcatca

SEQ ID NO: 5 - DNA, 24 bp, Artificial Sequence
  Synthetic primer
gtcccgccgaatccccgcaaacag

SEQ ID NO: 6 - DNA, 24 bp, Artificial Sequence
  Synthetic primer
tgaacacagacgctatgcgctcag

SEQ ID NO: 7 - DNA, 25 bp, Artificial Sequence
  Synthetic primer
cacctttatgtgagtggacacagag

SEQ ID NO: 8 - DNA, 25 bp, Artificial Sequence
  Synthetic primer
agaccctttgaagtcaaggacaccg

SEQ ID NO: 9 - DNA, 25 bp, Artificial Sequence
  Synthetic primer
ccattgctgaagaccttagtgatgc

SEQ ID NO: 10 - DNA, 20 bp, Artificial Sequence
  Synthetic primer
tctatgagggctagcctttg

SEQ ID NO: 11 - DNA, 20 bp, Artificial Sequence
  Synthetic primer
cctgactggaaggtagatgg

(19) United States
(12) Patent Application Publication
(10) Pub. No.: US 2012/0121546 A1
(43) Pub. Date: May 17, 2012

(54) Method of Producing Progenitor Cells from Differentiated Cells

(76) Inventor: Vishal Bhasin, Wellington (NZ)

(21) Appl. No.: 13/146,584

(22) PCT Filed: Feb. 5, 2010
(86) PCT No.: PCT/AU10/00120

§ 371 Date: Dec. 12, 2011

(30) Foreign Application Priority Data
Feb. 5, 2009 (AU) 2009900435
Feb. 5, 2009 (AU) 2009900436
Feb. 5, 2009 (AU) 2009900437
Feb. 5, 2009 (AU) 2009900438
Sep. 22, 2009 (AU) 2009904598
Sep. 22, 2009 (AU) 2009904599
Sep. 22, 2009 (AU) 2009904600
Sep. 22, 2009 (AU) 2009904601

(51) Int. Cl. A61K 35/12; C12N 5/071

(52) U.S. Cl. 424/93.7; 435/377

(57) ABSTRACT

The present invention provides a method of producing progenitor cells, such as cells capable of being differentiated into a plurality of different cell types, from differentiated cells. Methods of using progenitor cells in differentiation and/or tissue or organ repair and/or regeneration and/or building are also provides. Methods of using progenitor cells in treatment and prophylaxis of conditions alleviated by administering stem cells or tissue or organ derived from stem cells to a subject or by grafting stem cells or tissue or organ derived from stem cells into a subject or by transplanting stem cells or tissue or organ derived from stem cells into a subject are also provided. Also included are progenitor cells and differentiated cells and/or tissues and/or organs derived therefrom, and kits comprising same.

### RELATED APPLICATIONS

This application claims priority from Australian Patent Application Nos: AU 2009900435 filed Feb. 5, 2009; AU 200990436, filed Feb. 5, 2009; AU 2009900437 filed Feb. 5, 2009; AU 2009900438 filed Feb. 5, 2009; AU 2009904598 filed Sep. 22, 2009; AU 2009904599 filed Sep. 22, 2009; 2009904560 filed Sep. 22, 2009; and 2009904561 filed Sep. 22, 2009, the contents of which are incorporated herein by reference in their entirety.

### FIELD OF THE INVENTION

The present invention is in the field of medicinal cell biology and more particularly to cell culture, especially the culture of primary cells and cell lines that are differentiated or terminally differentiated. The present invention also relates to methods for producing cells with the ability to differentiate into a plurality of cell types such as for use in medicine and/or veterinary applications and/or for animal improvement.

### BACKGROUND OF THE INVENTION

The utility of stem cells (SCs), including hematopoietic SCs, mesenchymal SCs or multipotent adult progenitor cells such as endothelial progenitor cells (EPCs) and embryonic stem cells (ESCs), is well established, especially for generating multiple distinct cell types in medicine and/or veterinary applications and/or for animal improvement.

In particular, stem cells may be used as a source of cells that can be differentiated into various cell types to repopulate damaged cells. For example, joint pain is a major cause of disability, which most often results from damage to the articular cartilage by trauma or degenerative joint diseases such as primary osteoarthritis. Current methods of treatment for cartilage damage are often not successful in regenerating cartilage tissue to a fully functional state, and there is often considerable donor-site rejection. A resolution of this disease state can be provided by regenerating cartilage tissue using stem cells. There are many other tissue degenerative diseases, which can be treated using stem cells, including autoimmune disorders. For example, in the treatment and/or therapy of diabetes, the pancreatic islet cells of a diabetic patient can be regenerated using stem cells that are implanted and/or infused into the patient.

Despite the pluripotency of embryonic stem (ES) cells, legal and moral controversies concerning their use, and the lack of available human ES lines, have prompted researchers to turn to investigating new sources for isolating stem cells from tissues that are not of fetal origin. However, such adult stem cells still involve complicated isolation procedures, and are in limited supply.

Because of the numerous obstacles and technical difficulties in producing and using ES cells and adult stem cells in sufficient quantity for a large number of clinical applications, many researchers are now looking to develop strategies to reprogram somatic cells from adult tissues to thereby create cells having stem cell-like attributes, in particular the ability to differentiate into different cell types.

In one approach, mature cells are fused with embryonic germ cells by a process known as somatic-cell nuclear transfer (SCNT). After fusion, mature cell nuclei display pluripotent properties similar to that of the embryonic germ cells (Tada et al., 1997, EMBO J. 16:6510-6520). This fusion-process essentially returns the mature adult cell to an earlier developmental state (immature state), from which the cell can then mature into differentiated cell types. However, such reprogramming does not escape the requirement for isolated ES-cells or embryonic germ cells. Moreover, the ethical and religious issues associated with using human embryos apply equally to this technology. There are also practical difficulties in SCNT, including the short supply of human oocytes for SCNT.

In another approach, the sequential exposure of primary oligodendrocyte precursor cells (OPCs) to fetal calf serum and basic fibroblast growth factor (bFGF) produces cells that resemble multipotent stem cells (Kondo et al., 2000, Science 289:1754-1757). However, the procedure has not been shown to be applicable to other cells types and, as OPCs are not an abundant cell type, there is limited prospect for the large-scale application of this technology.

Finally, human fibroblasts have been shown to be capable of being made into pluripotent cells by ectopic expression of four factors: Oct3/4, Sox2, Klf4, and c-Myc (Kzutoshi et al., Cell 131:861-872 (2007); Park et al., Nature epub (2007)). The so-called “induced pluripotent stem cells” (iPSCs) produced by this technology were shown to be similar to human embryonic stem (ES) cells in morphology, proliferation, surface antigens, gene expression, epigenetic status of pluripotent cell-specific genes, and telomerase activity. On the other hand, the iPSCs were also shown to give rise to teratomas, raising concerns about the application of the technology to medicine and/or the veterinary industry and/or for animal improvement.

Accordingly, there is a need in the art for an abundant source of cells that are capable of being differentiated into different cell types without extracting or using egg cells or stem cells such as ES cells or the like, and with minimal deleterious effects. More particularly, there is a need in the art for alternative and/or improved methods of culturing differentiated cells and culture media suitable for producing cells capable of differentiating into a plurality of cells types, and which are preferably capable of supporting self-renewal of cells having this capacity. There is also a need for culture systems that permit maintenance of cells capable of differentiating into a plurality of cells types in vitro until the cells are required for subsequent cell or tissue regeneration or repair.

### SUMMARY OF THE INVENTION

In work leading up to the present invention, the inventor sought to identify conditions for producing cells having the ability to differentiate into multiple cell types i.e., that could be used as a source of different cell types in a similar manner to mesenchymal stem cells. Against conventional wisdom in the art, the inventor reasoned that the de-differentiation of already-differentiated cells might provide an abundant source of such cells for medical applications e.g., as an “off-the-shelf” supply of stem cell-like cells. The inventor also went against conventional wisdom in not merely seeking to expand existing populations of stems cells from primary tissues, by using differentiated cells as starting material.

As exemplified herein, the inventor has shown that it is possible to produce a cell having the ability to differentiate into different cell types by culturing human fibroblasts then detaching the cells.

The inventor has also shown that it is possible to produce a cell having the ability to differentiate into a different cell type by culturing human fibroblasts then detaching the cells and incubating the cells under high cell density conditions in a high density plating medium before adherence of the cells compared to standard culturing conditions where cells are incubated under standard cell density conditions e.g., at concentrations of about or below 20,000 cells per standard size culture well/plate. The inventor has also reasoned that it is possible to produce a cell having the ability to differentiate into a different cell type by culturing human fibroblasts in media comprising a modulator of 5′AMP-activated protein kinase or AMPK, compared to standard culture medium without a modulator of 5′AMP-activated protein kinase or AMPK. The inventor has also reasoned that it is possible to produce a cell having the ability to differentiate into a different cell type by culturing human fibroblasts in a medium comprising a phorbol ester or active derivative thereof, compared to standard culture medium without a phorbol ester or active derivative thereof. The inventor has further reasoned that it is possible to produce a cell having the ability to differentiate into a different cell type by culturing human fibroblasts in media comprising a retinoid, compared to standard culture medium without a retinoid.

Accordingly, in one example, the invention provides a method for producing a progenitor cell that is capable of being differentiated into a plurality of different cell types, said method comprising incubating or culturing differentiated cells and detaching the cells e.g., by incubating the cells in detachment medium comprising a protease or a ligand of a protease activated receptor (PAR). According to one such example, detaching the cells is induces trans-differentiation of the differentiated cells into the progenitor cells. In one example, the progenitor cells produced by this method are capable of being differentiated into a plurality of different cell types until re-attachment or adherence of the cells to the culture vessel and/or to each other. Alternatively or in, addition, the progenitor cells produced by this method are capable of being differentiated into a plurality of different cell types until contact to the culture vessel and/or to each other.

Accordingly, in another example, the present invention also provides a method for producing a progenitor cell that is capable of being differentiated into a plurality of different cell types, said method comprising incubating differentiated cells under high cell density conditions in a high density plating medium and detaching the cells e.g., by incubating the cells in detachment medium comprising a protease or a ligand of a protease activated receptor (PAR).

According to this example, the order of detachment and incubation of the cells under high density conditions in high density plating medium is not necessarily essential to the production of cells capable of undergoing subsequent differentiation into a plurality of different cell types. Conveniently, the differentiated cells are incubated in media comprising serum or with supplementation of factors normally present in serum for at least 2 days before detachment of the cells e.g., by incubating the cells in detachment media containing a protease or a ligand of a protease activated receptor (PAR). Conveniently, the differentiated cells are incubated in media containing serum and a medium containing a protease or a ligand of a protease activated receptor (PAR) before incubating the cells under high density conditions. Also where high density conditions are used, conveniently the detached cells are incubated under high density conditions before adherence directly in a high density plating medium.

As used herein, “detachment” or variations such as “detaching the cells” shall be taken to include any method of detaching cells from each other and/or from a surface of a culture vessel in which they are maintained known in the art. In one example, the cells are incubated in detachment medium comprising protease or PAR ligand for a time and under conditions sufficient for the cells to detach from each other and/or from a surface of a culture vessel in which they are maintained or to become rounder in appearance. By “PAR ligand” or equivalent term is meant a ligand capable of activating a protease-activated receptor, such as PAR-1 and/or PAR-2 and/or PAR-3 and/or PAR4. Without being bound by any theory or mode of action, detachment of cells e.g., by incubation in detachment medium comprising a protease or PAR ligand for a time and under conditions sufficient to detach the cells, or for their appearance to be modified in this manner, is sufficient for a partial or complete breakdown of integrins that normally mediate cell adhesion or at least for the promotion of cellular signalling pathways mediated by an integrin.

Alternatively, or in addition, the cells are incubated in the presence of a protease or PAR ligand for a time and under conditions sufficient for activation of one or more protease-activated receptors (PARs) such as PAR-1 and/or PAR-2 and/or PAR-3 and/or PAR4 to occur.

Preferred proteases and PAR ligands for performing the invention include chymotrypsin, trypsin, thrombin, pepsin, papain, matrix-metalloproteinase (MMP) and a PAR-2-activating peptide comprising the sequence SLIGRL. More preferably, the protease is trypsin, thrombin, plasmin, or a PAR-2-activating peptide comprising the sequence SLIGRL. In a particularly preferred example, trypsin is employed.

In another example, the cells are detached from each other and/or from a surface of a culture vessel in which they are maintained or become rounder in appearance by incubating the cells in a Ca²⁺-free and Mg⁺-free detachment medium comprising ethylenediaminetetraacetic acid (EDTA) for a time and under conditions sufficient for detachment of integins from the cellular matrix. In a further example, cells are detached from each other and/or from a surface of a culture vessel in which they are maintained or become rounder in appearance by incubating the cells in a detachment medium comprising citric saline for a time and under conditions sufficient for detachment of the cells and/or integins from the cellular matrix.

As used herein, the term “high density” or similar term such as “high density conditions” or “high cell density conditions” shall be taken to mean that the cells are maintained, cultured or incubated until confluence or cell-to-cell contact is achieved or at a starting density of cells of about 50,000 cells to about 200,000 cells per standard-size culture well/plate, including about 60,000 cells or greater per standard-size culture well/plate, or about 70,000 cells or greater per standard-size culture well/plate, or about 80,000 cells or greater per standard-size culture well/plate, or about 90,000 cells or greater per standard-size culture well/plate, or about 100,000 cells or greater per standard-size culture well/plate, or about 200,000 cells per standard-size culture well/plate. Higher cell densities above about 200,000 cells per standard-size culture well/plate may also be employed. By “standard-size” in this context is meant about 27 mm² plating surface area in a well or plate:

Alternatively or in addition, high density conditions include the maintenance, culture or incubation of cells at a starting density of cells of about 1500 cells/mm² plating surface area to about 10,000 cells/mm² plating surface area, including about 1,850 cells/mm² surface area of the culture vessel or greater, or about 2,220 cells/mm² surface area of the culture vessel or greater, or about 2,590 cells/mm² surface area of the culture vessel or greater, or about 2,960 cells/mm² surface area of the culture vessel or greater, or about 2,220 cells/mm² surface area of the culture vessel or greater, or about 3,330 cells/mm² surface area of the culture vessel or greater, or about 3,703 cells/mm² surface area of the culture vessel surface area of the culture vessel or greater, or about 7,407 cells/mm² surface area of the culture vessel surface area of the culture vessel or greater.

The term “high density medium” or “high density plating medium” means any cell medium capable of supporting progenitor cells produced by the method of the present invention. In one example progenitor cells produced by the method of the invention undergo minimal or no cell division when cultured, maintained or incubated in the high density plating medium. Exemplary high density plating medium includes Medium-199 comprising 170 nM insulin, 0.5 mM 3-isobutyl-1-methylxanthine, 0.2 mM indomethacin, 1 μM dexamethasone, and 15% rabbit serum. Alternatively, high density plating medium includes Dulbecco's Modified Eagle Medium (DMEM) or basal Medium 199 supplemented with 10% fetal calf serum (FCS). However other media may be employed.

The optimum period of maintenance, culture or incubation in high density plating medium is determined empirically e.g., by calculating the optimum number of differentiated cells produced from aliquots of progenitor cells incubated at high density over a time course and subsequently incubated under conditions sufficient for differentiation to occur. Alternatively, or in addition, the optimum period of maintenance, culture or incubation in high density plating medium is determined empirically e.g., by determining fibroblast-specific and/or progenitor cell-specific marker expression by aliquots of progenitor cells incubated at high density over a time course. Alternatively, or in addition, the optimum period of maintenance, culture or incubation in high density plating medium is a period of time until adherence is achieved, i.e., a shorter time than required for cells to become adherent. Alternatively, or in addition, the optimum period of maintenance, culture or incubation in high density plating medium is up to about 5 days, including up to about 4 days or up to about 3 days or up to about 2 days or up to about 1 day i.e., up to about 24 hours.

According to one example, the cells are detached and optionally incubated under high cell density conditions in a high density plating medium for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division.

In one example of the invention, cells are introduced to high density culture conditions within about 6 hours to about 10 hours from their detachment, including within about 6 hours to about 9 hours from their detachment, or within about 6 hours to about 8 hours from their detachment, or within about 6 hours to about 7 hours from their detachment. In another example, cells are introduced to high density culture conditions within about 1 hour to about 6 hours from their detachment, including within about 5 hours to about 6 hours from their detachment, or within about 4 hours to about 5 hours from their detachment, or within about 3 hours to about 4 hours from their detachment, or within about 2 hours to about 3 hours from their detachment, or within about 1 hours to about 2 hours from their detachment. In another example, cells are introduced to high density culture conditions in less than about 5 hours from their detachment, including less than about 4 hours from their detachment, or less than about 3 hours from their detachment, or less than about 2 hours from their detachment, or less than about 1 hour from their detachment. In yet another example, the differentiated cells are incubated in a low-serum media, subjected to one or more means of achieving their detachment, and simultaneously introduced to high density culture conditions.

In another example, cells are incubated under high density conditions e.g., until confluence or cell-to-cell contact is achieved, before detaching the cells. Such cells may be subsequently seeded at any density e.g., on a biocompatible matrix or in culture medium such as to promote their differentiation.

In one example the method of the present invention optionally further comprises incubating differentiated cells in media comprising a low serum concentration and without supplementation of factors normally present in serum. In one such example, the culture medium for the incubation of the differentiated cells may be a low-serum medium. The order of detachment, incubating differentiated cells under high cell density conditions in a high density plating medium and incubation in low serum medium is not necessarily essential to the production of cells capable of undergoing subsequent differentiation into a plurality of different cell types. Conveniently, the differentiated cells are incubated under high cell density conditions in a high density plating medium simultaneously with incubation in media comprising a low-serum concentration and without supplementation of factors normally present in serum before detachment. In one example, the differentiated cells are incubated in a high density plating medium and also comprising low serum concentration and without supplementation of factors normally present in serum before detachment.

The term “serum” means the non-cellular liquid phase of blood that remains after coagulation and removal of the blood clot, including blood cells, platelets and fibrinogen. The present invention is not to be limited by the nature of the serum used in low-serum media, the only requirement being that the cells are able to maintain viability in the medium used. In this respect, it is known that normal human fibroblasts require growth factors provided e.g., by fetal bovine serum (FBS) or fetal calf serum (FCS) at about 10% (v/v) for proliferation in culture.

In one example, preferred sera for cell culture are bovine sera e.g., fetal calf serum and fetal bovine serum. Horse sera or artificial sera comprising the constituents of naturally-occurring sera from these sources may also be employed. In another example, in using the cells of the invention in human therapy, preferred sera for cell culture is human sera or artificial sera comprising the constituents of naturally-occurring human sera.

As used herein, the term “low serum concentration” shall be taken to mean a concentration of serum not exceeding about 3% (v/v) in culture medium, preferably not exceeding about 2% (v/v) or about 1% (v/v), and still more preferably, less than 1% (v/v) serum concentration, including serum-free or no serum. Unless the context requires otherwise e.g., by virtue of the addition of a growth factor agonist of the Akt/(PKB) pathway and/or NF-κB pathway, the term “low-serum” shall also be taken to mean conditions in which the concentration of a growth factor supplement in the culture medium is at a level equivalent to or less than the level of the growth factor in serum. In the present context, an alternative low-serum medium includes “artificial sera” or “depleted sera” having low levels of growth factors required for cellular proliferation.

In a particularly preferred example, the term “low serum concentration” shall be taken to mean a concentration of serum between about 0% (v/v) and about 1% serum concentration or an artificial serum or depleted serum having an equivalent or lower level of one or more serum growth factors. Standard methods in cell biology are used to determine the parameters for what constitutes a particular concentration of any serum, including fetal calf serum and bovine serum.

Particularly preferred low-serum media for incubation of the differentiated cells are Dulbecco's Modified Eagle Medium High Glucose (DMEM-HG; e.g., Lonza Cat #12-604), or basal Medium 199 or a modified Medium 199 containing high glucose. Preferably, the low-serum medium comprises one or more sugars such as glucose, at a concentration of at least about 0.1% (w/v), more preferably at least about 0.2% (w/v) or at least about 0.3% (w/v) or at least about 0.4% (w/v) or at least about 0.5% (w/v) or at least about 0.6% (w/v) or at least about 0.7% (w/v) or at least about 0.8% (w/v) or at least about 0.9% (w/v) or at least about 1.0% (w/v).

Optionally, when the differentiated cells are incubated in a low-serum medium preferably the differentiated cells are incubated in low-serum media for at least about two days i.e., about 48 hours, and not exceeding about ten days i.e., about 240 hours, including for about two days or about three days or about four days or about five days or about six days or about seven days or about eight days or about nine days or about ten days. More preferably, the cells are incubated in low-serum media for a period between about four days and about nine days, including about four days or about five days or about six days or about seven days or about eight days or about nine days. Still more preferably, the cells are incubated in low-serum media for a period between about five days and about eight days, including about five days or about six days or about seven days or about eight days. As will be apparent from the disclosure herein, lower numbers of progenitor cells may be apparent with shorter periods of exposure of the cells to low serum media than are observed for optimum periods of incubation in low serum media, however such sub-optimum incubation conditions are clearly within the scope of the invention.

Alternatively, or in addition, the cells are incubated in low-serum medium for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division.

Accordingly, in one example, the present invention also provides a method for producing a progenitor cell that is capable of being differentiated into a plurality of different cell types, said method comprising incubating differentiated cells in media comprising a modulator of 5′AMP-activated protein kinase or AMPK and detaching the cells e.g., by incubating the cells in a detachment medium containing a protease or a ligand of a protease activated receptor (PAR).

The order of incubation in the presence of a modulator of 5′AMP-activated protein kinase or AMPK and detachment is not necessarily essential to the production of cells capable of undergoing subsequent differentiation into a plurality of different cell types. Conveniently, the differentiated cells are incubated in media comprising a modulator of 5′AMP-activated protein kinase or AMPK before performing detachment.

Without being bound by any theory or mode of action, Adenosine 5′-monophosphate-activated protein kinase or 5′AMP-activated protein kinase or AMPK is a heterotrimeric protein kinase that plays a role in cellular energy homoeostasis and is thought to become activated when phosphorylation takes place at threonine-172 (Thr-172) residue in response to changes in cellular ATP levels. In one example, a modulator of 5′AMP-activated protein kinase or AMPK activates and/or enhances function of 5′AMP-activated protein kinase or AMPK or activates and/or enhances one or more AMPK signalling pathway(s). According to this example, the modulator may include an agonist and/or a partial agonist and/or a reverse antagonist of 5′AMP-activated protein kinase or AMPK. In another example, a modulator of 5′AMP-activated protein kinase or AMPK suppresses and/or inhibits function of 5′AMP-activated protein kinase or AMPK or suppresses and/or inhibits one or more AMPK signalling pathway(s). According to this example the modulator may include an antagonist and/or a partial antagonist and/or a reverse agonist of TAMP-activated protein kinase or AMPK.

Preferably, the cells are incubated in the presence of a modulator of 5′AMP-activated protein kinase or AMPK to achieve optimum plasticity and/or multipotency or pluripotency. Such incubation is preferably for a time and under conditions sufficient to induce and/or activate 5′AMP-activated protein kinase or AMPK or a component thereof that is sufficient to render the cells capable of being differentiated into a plurality of different cell types. Alternatively, the cells are incubated in the presence of a modulator of 5′AMP-activated protein kinase or AMPK for a time and under conditions sufficient to inhibit and/or suppress 5′AMP-activated protein kinase or AMPK or a component thereof that is sufficient to render the cells capable of being differentiated into a plurality of different cell types.

Alternatively, or in addition, the cells are incubated in the presence of a modulator of 5′AMP-activated protein kinase or AMPK for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division. Alternatively, or in addition, the cells are incubated in the presence of a modulator of 5′AMP-activated protein kinase or AMPK for a period of time sufficient for phosphorylation and/or activation and/or stabilization of tumor suppressor p53 protein that delays or inhibits or represses cell cycle progression or cell division.

Preferred modulators of 5′AMP-activated protein kinase or AMPK suitable for this purposes are described herein and include but not limited to e.g., AICAR [5-aminoimidazole-4-carboxamide-1-β-4-ribofuranoside], a phosphorylated AICAR-riboside or ZMP [5-aminoimidazole-4-carboxamide-ribotide], Metformin (Glucophage) [1,1-dimethylbiguanide], an appetite-stimulating hormone ghrelin/obestatin prepropeptide (GHRL), 3PG [3-Phosphoglyceric acid], thrombin, extracellular AMP [5′-adenosine monophosphate], long chain fatty acyl analogs such as acyl-CoA thioester, or Compound C or Dorsomorphin (6-[4-(2-Piperidin-1-yl-ethoxy)-phenyl)]-3-pyridin-4-yl-pyrrazolo[1,5-a]-pyrimidine), glycogen, or PPARα agonist (αA) and PPARα/γ dual agonist or phosphocreatine.

In one example the method of the present invention optionally further comprises incubating differentiated cells in media comprising a low serum concentration and without supplementation of factors normally present in serum. The order of incubating differentiated cells in media comprising a modulator of 5′AMP-activated protein kinase or AMPK, detachment and incubation in low serum medium is not necessarily essential to the production of cells capable of undergoing subsequent differentiation into a plurality of different cell types. Conveniently, the differentiated cells are incubated in media comprising a modulator of 5′AMP-activated protein kinase or AMPK simultaneously with incubation in media comprising a low-serum concentration and without supplementation of factors normally present in serum before detachment. In one example, the differentiated cells are incubated in media comprising a modulator of 5′AMP-activated protein kinase or AMPK and also comprising low serum concentration and without supplementation of factors normally present in serum before detachment.

In one example, the method of the present invention further comprises incubating the cells under high cell-density conditions. In accordance with this example, a high density plating medium is employed as described above. In one example progenitor cells produced by the method of the invention undergo minimal or no cell division when cultured, maintained or incubated in high density plating medium. Exemplary high density plating medium includes Medium-199 comprising 170 nM insulin, 0.5 mM 3-isobutyl-1-methylxanthine, 0.2 mM indomethacin, 1 μM dexamethasone, and 15% rabbit serum. Alternatively, high density plating medium includes Dulbecco's Modified Eagle Medium (DMEM) or basal Medium 199 supplemented with 10% fetal calf serum (FCS). However other media may be employed.

The order of incubation in media comprising a modulator of 5′AMP-activated protein kinase or AMPK and detachment and optionally incubating the differentiated cells in low-serum medium and optionally incubating the cells under high cell density conditions is not necessarily essential to the production of progenitor cells capable of undergoing subsequent differentiation into a plurality of different cell types. Conveniently, the differentiated cells are incubated in the presence of a modulator of 5′AMP-activated protein kinase or AMPK optionally in a low-serum media, and subjected to one or more means of achieving their detachment, before being incubated under high-cell density conditions.

An advantage of incubating cells at high cell density conditions in concert with incubation in the presence of a modulator of 5′AMP-activated protein kinase or AMPK and detachment of the cells, is that the proportion of progenitor cells capable of being differentiated into a plurality of different cell types is increased.

Accordingly, in one example, the present invention provides a method for producing a progenitor cell that is capable of being differentiated into a plurality of different cell types, said method comprising incubating differentiated cells in a medium comprising a phorbol ester or active derivative thereof for a time and under conditions sufficient to produce a progenitor cell that is capable of being differentiated into a plurality of different cell types.

In another example, the present invention also provides a method for producing a progenitor cell that is capable of being differentiated into a plurality of different cell types, said method comprising incubating differentiated cells in a medium comprising a phorbol ester or active derivative thereof and detaching the cells e.g., by incubating the cells in media containing a protease or a ligand of a protease activated receptor (PAR).

The order of incubation in the presence of a phorbol ester or an active derivative thereof and detachment is not necessarily essential to the production of cells capable of undergoing subsequent differentiation into a plurality of different cell types. Conveniently, the differentiated cells are incubated in media comprising a phorbol ester or an active derivative thereof before performing detachment.

In one example, the present invention comprising incubating differentiated cells with an agent comprising as an active ingredient a phorbol ester derivative of formula (I):

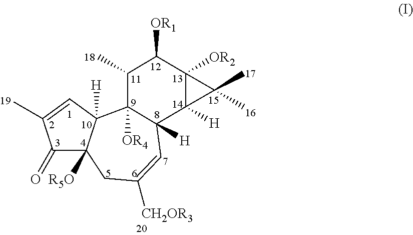

Wherein R₁, R₂, R₃, R₄, and R₅, independently of one another, represent a hydrogen atom, an aliphatic carboxylic acid residue, or an aromatic carboxylic acid residue; and wherein the phorbol ester derivative induces trans-differentiation of the differentiated cells into progenitor cells capable of differentiating into different cell types.

In another example, the present invention comprising incubating differentiated cells with an agent comprising as an active ingredient a phorbol ester derivative of formula (I):

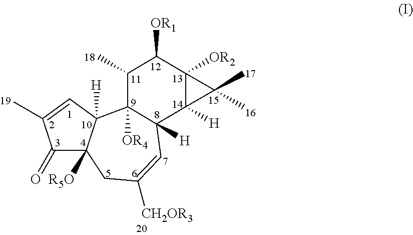

wherein R₁ is a hydrogen, or a butyryl, or a decanoyl, or a tetradecanoyl, or a N-methylaminobenzoyl group; R₂ is a formyl, or acetyl, or propionyl, or butyryl or pentanoyl, or hexanoyl, or benzoyl, or phenylacetyl group; R₃ is hydrogen or linoleic acid; R₄, and R₅ are each hydrogen.

In another example, the present invention comprising incubating differentiated cells with an agent comprising as an active ingredient a phorbol ester derivative of formula (I):

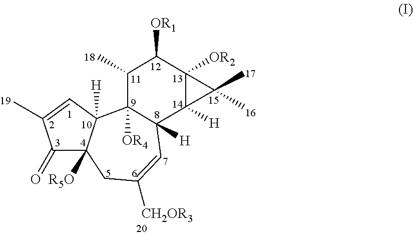

wherein R₁ is hydrogen, or butyryl; R₂ is a formyl, or acetyl, or propionyl, or butyryl or pentanoyl, or hexanoyl, or benzoyl, or phenylacetyl group; R₃, R₄, and R₅ are each hydrogen.

In another example, the present invention comprising incubating differentiated cells with an agent comprising as an active ingredient a phorbol ester derivative of formula (I):

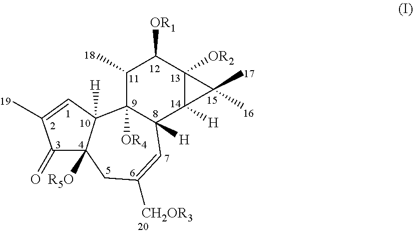

wherein R₁ is hydrogen, or butyryl; R₂ is acetyl, or butyryl; and R₃, R₄, and R₅ are each hydrogen.

In another example, the present invention comprising incubating differentiated cells with an agent comprising as an active ingredient 4β-12-O-tetradecanoylphorbol-13-acetate (PMA or TPA) or a stereo-isomer thereof.

In another example, the present invention comprising incubating differentiated cells with an agent comprising as an active ingredient 4β-phorbol-12,13-dibutyrate (PDBu) or a stereo-isomer thereof.

In another example, the present invention comprising incubating differentiated cells with an agent comprising as an active ingredient 12-O-[2-methylaminobenzoate]-4-deoxy-13-acetate-14-deoxy phorbol (phorbol sapintoxin A) or a stereo-isomer thereof.

In another example, the present invention comprising incubating differentiated cells with an agent comprising as an active ingredient 12-O-[2-methylaminobenzoate]-4-hydroxy-13-acetate-14-deoxy phorbol (phorbol sapintoxin D) or a stereo-isomer thereof.

In yet another example, the present invention comprising incubating differentiated cells with an agent comprising as an active ingredient a phorbol ester or an active derivative thereof that mimics the action i.e., is an analogue of diacylglycerol (DAG).

In yet another example, the present invention comprising incubating cells with an agent comprising as an active ingredient a phorbol ester or an active derivative thereof that activates or induces protein kinase C(PKC).

The term “active derivative thereof” shall be taken to mean any natural or synthetic structural derivative of a phorbol ester that is capable of inducing trans-differentiation of differentiated cells into progenitor cells capable of differentiating into other cell types. Such “derivatives”, or their functional equivalents, may be naturally occurring and isolated by means known to those skilled in the art, such as, for example as described in Goel et al., Int J Toxicol 26:279-288 (2007) or any references described therein, and is incorporated herein by reference. Alternatively, or in addition such derivatives”, or their functional equivalents may be generated chemically or synthetically by several means known to those skilled in the art, such as, for example as described in U.S. Pat. No. 6,268,395 or any references described therein and incorporated herein by reference.

Preferably, the cells are incubated in the presence of a phorbol ester or active derivative thereof according to any example described herein, to achieve optimum plasticity and/or multipotency or pluripotency. In one non-limiting example such incubation is for a time and under conditions sufficient to induce and/or activate PKC or a component thereof and/or Akt/protein kinase B (PKB) or a component thereof and/or the transcriptional regulator nuclear factor kappa-light-chain-enhancer of activated B cells (NF-kB) or a component thereof and/or activator protein 1 (AP1) or a component thereof that is sufficient to render the cells capable of being differentiated into a plurality of different cell types. Alternatively or in addition, the cells are incubated in the presence of a phorbol ester or active derivative thereof for a time and under conditions sufficient to render the cells capable of being differentiated into a plurality of different cell types.

Alternatively, or in addition, the cells are incubated in the presence of a phorbol ester or active derivative thereof for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division.

In one example the method of the present invention optionally further comprises incubating differentiated cells in media comprising a low serum concentration and without supplementation of factors normally present in serum. The order of incubating differentiated cells in media comprising phorbol ester or an active derivative thereof and detaching the cells preferably by incubating the cells in media containing a protease or a ligand of a protease activated receptor (PAR) and incubation in low serum medium is not necessarily essential to the production of cells capable of undergoing subsequent differentiation into a plurality of different cell types. Conveniently, the differentiated cells are incubated in media comprising low serum concentration and without supplementation of factors normally present in serum before incubation with a phorbol ester or an active derivative thereof and before detachment. In another example, the differentiated cells are incubated in media comprising phorbol ester or an active derivative thereof and also comprising low serum concentration and without supplementation of factors normally present in serum before detachment.

In one example, the method of the present invention further comprises incubating the cells under high cell-density conditions. In accordance with this example, a high density plating medium is employed. In one example progenitor cells produced by the method of the invention undergo minimal or no cell division when cultured, maintained or incubated in the high density plating medium. Exemplary high density plating medium includes Medium-199 comprising 170 nM insulin, 0.5 mM 3-isobutyl-1-methylxanthine, 0.2 mM indomethacin, 1 μM dexamethasone, and 15% rabbit serum. Alternatively, high density plating medium includes Dulbecco's Modified Eagle Medium (DMEM) or basal Medium 199 supplemented with 10% fetal calf serum (FCS). However other media may be employed.

The order of incubation in media comprising phorbol ester or active derivative thereof and detachment and optionally incubating the differentiated cells in low-serum medium and optionally incubating the cells under high cell density conditions is not necessarily essential to the production of progenitor cells capable of undergoing subsequent differentiation into a plurality of different cell types. Conveniently, the differentiated cells are incubated in the presence of a phorbol ester or active derivative thereof optionally in a low-serum media, and subjected to one or more means of achieving their detachment, before being incubated under high-cell density conditions. An advantage of incubating cells at high cell density conditions in concert with incubation in the presence of a phorbol ester or active derivative thereof and detachment of the cells, is that the proportion of progenitor cells capable of being differentiated into a plurality of different cell types is increased.

In another example, the present invention also provides a method for producing a progenitor cell that is capable of being differentiated into a plurality of different cell types, said method comprising incubating differentiated cells in media comprising a retinoid and detaching the cells preferably by incubating the cells in a medium containing a protease or a ligand of a protease activated receptor (PAR). The order of incubation in the presence of a retinoid and detachment is not necessarily essential to the production of cells capable of undergoing subsequent differentiation into a plurality of different cell types. Conveniently, the differentiated cells are incubated in media comprising a retinoid before performing detachment.

As used herein the term retinoid shall be taken to include a retinoic acid including any stereo-isomer of retinoic acid such as all-trans-retinoic acid (ATRA) or 9-cis retinoic acid (9CRA), or 13-cis retinoic acid or 11-cis retinoic acid or an analogue of a retinoic acid. It will be understood that the present invention includes a retinoid that is naturally occurring or synthetic and is known in the art or to be developed in the future.

As used herein an analogue of a retinoic acid shall be taken to mean any compound capable of binding to a retinoic acid receptor or ligand or any compound that is capable of agonising a receptor or ligand of retinoic acid or any compound that is capable of antagonising a receptor or ligand of a retinoic acid or any compound that is capable of mimicking modulation e.g., agonism and/or antagonism of a receptor or ligand of a retinoic acid.

In one example, the agonism or antagonism of a receptor of retinoic acid or mimicking modulation of a receptor or ligand of a retinoic acid includes modulating expression of one or more retinoic acid responsive or dependent gene(s). In one example, modulating expression of retinoic acid responsive or dependent gene(s) includes binding of a retinoic acid receptor or ligand to transcription regulating element(s) or region(s) of a retinoic acid target gene(s) thereby modulating transcription of one or more retinoic acid responsive or dependent gene(s).

In one example, the present invention comprising incubating cells with a retinoid that modulates a retinoic acid receptor (RAR) or any isoform thereof. Preferably, the present invention comprising incubating cells with a retinoid that agonises a retinoic acid receptor (RAR) or any isoform thereof. Alternatively, or in addition thereto, the present invention comprising incubating cells with a retinoid that modulates a retinoid X receptor (RXR) or any isoform thereof. Preferably, the present invention comprising incubating cells with a retinoid that agonises a retinoid X receptor (RXR) or any isoform thereof. Alternatively, or in addition thereto, the present invention comprising incubating cells with a retinoid that modulates a cellular retinoic acid binding protein (CRABP) or an isoform thereof. Preferably, the present invention comprising incubating cells with a retinoid that agonises a cellular retinoic acid binding protein (CRABP) or an isoform thereof.

Preferred retinoids for performing the invention include for example all-trans-retinoic acid (ATRA), 9-cis retinoic acid (9CRA), 13-cis retinoic acid, 11-cis retinoic acid, Am80, BMS189452, CD666, BMS188649, BMS185411, BMS188649, CD336/Am580, CD2019, CD437/AHPN, CD2665, CD2503, CD367, CD2314, CD 3640, AGN193109.

In one example, the present invention comprises incubating differentiated cells with ATRA. In another example, the present invention comprises incubating differentiated cells with 9CRA. In another example, the present invention comprises incubating differentiated cells with Am80. In another example, the present invention comprises incubating differentiated cells with BMS188649. In another example, the present invention comprises incubating differentiated cells with CD336/Am580. In another example, the present invention comprises incubating differentiated cells with AGN193109.

According to one example, the present invention comprising incubating differentiated cells with a combination of two or more retinoids. Preferably, according to this example, incubation of differentiated cells with a combination of two or more retinoids has an enhanced or synergistic effect on production of producing progenitor cells capable of being differentiated into a plurality of different cell types than incubation of each of the retinoids in the combination separately.

In one example, the present invention comprises incubating differentiated cells with a combination of two or more retinoids, wherein each of the retinoids is selected form a group comprising all-trans-retinoic acid (ATRA), 9-cis retinoic acid (9CRA), 13-cis retinoic acid, 11-cis retinoic acid, Am80, BMS189452, CD666, BMS188649, BMS185411, BMS188649, CD336/Am580, CD2019, CD437/AHPN, CD2665, CD2503, CD367, CD2314, CD 3640, AGN193109. Preferably, each of the retinoids in the combination is selected from a group comprising all-trans-retinoic acid (ATRA), 9-cis retinoic acid (9CRA), 13-cis retinoic acid, 11-cis retinoic acid, Am80, BMS189452, CD666, BMS188649, BMS185411, BMS188649, CD336/Am580, CD2019, CD437/AHPN, CD367, CD2314, CD 3640. In one example, the present invention comprises incubating differentiated cells with a combination of AM80 and preferably BMS188649.

In one example, the differentiated cells are incubated in a medium comprising a retinoid, wherein the retinoid is a constituent normally present in the medium or is a added to the medium. In one example, the cells are incubated in a medium comprising serum, wherein the serum naturally comprises the retinoid. In one example, the cells are incubated in a medium comprising serum between about 5% (v/v) to about 50% (v/v), including a medium comprising serum at about 6% (v/v), or serum at about 7% (v/v), or serum at about 8% (v/v), or serum at about 9% (v/v), or serum at about 10% (v/v), or serum at about 15% (v/v), or serum at about 20% (v/v), or serum at about 25% (v/v), serum at about 30% (v/v), serum at about 35% (v/v), serum at about 40% (v/v), serum at about 45% (v/v), serum at about 50% (v/v).

Alternatively, the cells are incubated in a medium comprising a retinoid at a final concentration of about 10⁻¹⁰ M to about 10⁻² M, including final concentration of about 10⁻⁹M, or about 10⁻⁸M, or about 10⁻⁷M, or about 10⁻⁶M, or about 10⁻⁵M or about 10⁻⁴ M or about 10⁻³M or about 10⁻²M in the medium.

In one example, the differentiated cells are incubated in a medium comprising a retinoid for at least about one day i.e., about 24 hours to about 11 days i.e., about 264 hours, including for about two days, or about three days, or about four days, or about five days, or about six days, or about seven days, or about eight days, or about 10 days, or about 11 days. More preferably, the cells are incubated in a medium comprising a retinoid for a period between about two days and about nine days, including about three days or about four days, or about five days, or about seven days, or about eight days or about nine days. Still more preferably, the cells are incubated in medium comprising a retinoid for a period between about three days and about seven days, including about four days or about five days or about six days or about seven days. As will be apparent from the disclosure herein, lower numbers of progenitor cells may be produced with shorter periods of exposure of the cells to a retinoid than are observed for optimum periods of incubation in media comprising a retinoid, however such sub-optimum incubation conditions are clearly within the scope of the invention.

In one example, the cells are incubated in the presence of a retinoid according to any example described herein, to achieve optimum plasticity and/or multipotency or pluripotency. In one non-limiting example such incubation is for a time and under conditions sufficient to agonise or antagonise a retinoic acid receptor (RAR) or any isoform thereof, and/or agonise or antagonise retinoic X receptor (RXR) or any isoform thereof, and/or agonise or antagonise a cellular retinoic acid binding protein (CRABP) or any isoform thereof that is sufficient to render the cells capable of being differentiated into a plurality of different cell types. Alternatively or in addition, the cells are incubated in the presence of a retinoid for a time and under conditions sufficient to render the cells capable of being differentiated into a plurality of different cell types.

Alternatively, or in addition, the cells are incubated in the presence of a retinoid for a period of time sufficient for the level of one or more gene products of the cells that delay or inhibit or repress cell cycle progression or cell division to be expressed de novo or at an increased level in the cells, such as, for example, the cell cycle proteins p27^{Kip1} and/or p57^{Kip2} and/or p18. These proteins are expressed in fibroblasts and down-regulated before the onset of cell division.

In one example the method of the present invention optionally further comprises incubating differentiated cells in media comprising a low serum concentration and without supplementation of factors normally present in serum. The order of incubating differentiated cells in media comprising retinoid and detaching the cells preferably by incubating the cells in media containing a protease or a ligand of a protease activated receptor (PAR) and incubation in low serum medium is not necessarily essential to the production of cells capable of undergoing subsequent differentiation into a plurality of different cell types. Conveniently, the differentiated cells are incubated in media comprising low serum concentration and without supplementation of factors normally present in serum before incubation with a retinoid and before detachment. In another example, the differentiated cells are incubated in media comprising a retinoid and also comprising low serum concentration and without supplementation of factors normally present in serum before detachment.

In one example, the method of the present invention further comprises incubating the cells under high cell-density conditions. In accordance with this example, a high density plating medium is employed. In one example progenitor cells produced by the method of the invention undergo minimal or no cell division when cultured, maintained or incubated in the high density plating medium. Exemplary high density plating medium includes Medium-199 comprising 170 nM insulin, 0.5 mM 3-isobutyl-1-methylxanthine, 0.2 mM indomethacin, 1 μM dexamethasone, and 15% rabbit serum, however other media may be employed. Alternatively, high density plating medium includes Dulbecco's Modified Eagle Medium (DMEM) or basal Medium 199 supplemented with 10% fetal calf serum (FCS). However other media may be employed.

The order of incubation in media comprising a retinoid and detachment and optionally incubating the differentiated cells in low-serum medium and optionally incubating the cells under high cell density conditions is not necessarily essential to the production of progenitor cells capable of undergoing subsequent differentiation into a plurality of different cell types. Conveniently, the differentiated cells are incubated in the presence of a retinoid optionally in a low-serum media, and subjected to one or more means of achieving their detachment, before being incubated under high-cell density conditions. An advantage of incubating cells at high cell density conditions in concert with incubation in the presence of a retinoid and detachment of the cells, is that the proportion of progenitor cells capable of being differentiated into a plurality of different cell types is increased.

It will be apparent from the disclosure herein that similar, equivalent or improved results e.g., in terms of numbers of progenitor cells capable of being differentiated into a plurality of different cell types and/or in terms of the degree of multipotency and/or pluripotency of the progenitor cells produced, are able to be produced by agonism of the Akt/(PKB) pathway and/or NF-kB pathway. Without being bound by any theory or mode of action, the inventor reasoned that the detachment of cells and optionally high density maintenance, culture or incubation condition to induce optimum plasticity of fibroblasts coincides with induction of the Akt/(PKB) and/or NF-kB pathway(s), and that the responses of cells to the detachment of cells and optionally when detachment is combined with high density maintenance, culture or incubation condition are likely to induce one or both pathways. The inventor has also reasoned that incubation of the cells under high density conditions preferably before adherence of the cells to the vessel directly in a high density plating medium induces activation of the NF-kB pathway, possibly by cell-to-cell receptor signalling and/or by inducing the intracellular PKC or Ca²⁺ influx. Also without being bound by any theory or mode of action, the inventor has further reasoned that the incubation in the presence of a modulator of 5′AMP-activated protein kinase or AMPK and/or in the presence of a phorbol ester or active derivative thereof and/or in the presence of a retinoid and detachment, and optionally low-serum incubation and/or optionally high density maintenance, culture or incubation conditions to induce optimum plasticity of fibroblasts coincides with the induction of the Akt/(PKB) and/or NF-kB pathway(s), and that the responses of cells to the combined modulation of AMPK and/or in the presence of a phorbol ester or active derivative thereof and/or in the presence of a retinoid and detachment, and detachment and optionally low-serum incubation and/or optionally high density maintenance, culture or incubation conditions, are likely to induce one or both pathways.

Accordingly, an alternative example of the present invention provides a method for producing a progenitor cell that is capable of being differentiated into a plurality of different cell types, said method comprising incubating differentiated cells in media comprising an amount of an agonist or partial agonist of the Akt/(PKB) pathway and/or NF-kB pathway and for time sufficient to render the cells capable of being differentiated into a plurality of different cell types.

This example of the invention is performed on cultured cells ex vivo. In accordance with this example, it is preferred that the method comprises the first step of obtaining isolated cells from a suitable source e.g., from a commercial supplier.

Alternatively, the cells have been isolated previously from a human or animal subject, including a syngeneic subject to whom progenitor cells produced by the method can optionally be administered e.g., topically, systemically, locally as an injectable and/or transplant and/or device, or in conjunction with one or more treatments for injuries, allografts or autografts.

Alternatively, or in addition, an intermediate step of cell expansion in culture may be performed to increase the number of progenitor cells. Cell expansion in culture may be performed, for example, prior to administration of cells to a subject.

Preferred agonists of the Akt/(PKB) pathway suitable for this purpose are described herein and include e.g., interleukin-1 (IL-1), platelet derived growth factor (PGDF-BB), insulin growth factor (IGF-1), transforming growth factor-beta (TGF-β), nerve growth factor (NGF) and carbachol or any active fragment or active chemical group thereof.

Preferred agonists of the NF-κB pathway suitable for this purposes are described herein and include e.g., tumor necrosis factor-alpha (TNF-α), interleukin 1 (IL-1), or any active fragment thereof, lysophosphatidic acid (LPA) or lipopolysaccharide (LPS).

In one example, the cells are incubated in the presence of an agonist of the Akt/(PKB) pathway and/or NF-κB pathway in a culture medium as described according to any example hereof to achieve optimum plasticity and/or multipotency or pluripotency. Such incubation is preferably for a time and under conditions sufficient to induce the Akt/(PKB) pathway and/or NF-κB pathway in the cells or a component thereof that is sufficient to render the cells capable of being differentiated into a plurality of different cell types. In one such example, the cells maintained, cultured or incubated at high density conditions according to any example hereof and optionally additionally incubated in or maintained on low-serum medium or serum-free medium, and without supplementation of factors normally present in serum, e.g., before, during or following incubation in the presence of one or more agonists of the Akt/(PKB) pathway and/or one or more agonists of the NF-κB pathway. In another such example, the cells are further incubated in a medium comprising a modulator of 5′AMP-activated protein kinase or AMPK according to any example hereof and optionally additionally incubated in or maintained on low-serum medium, and without supplementation of factors normally present in serum, e.g., before, during or following incubation in the presence of one or more agonists of the Akt/(PKB) pathway and/or one or more agonists of the NF-κB pathway. In another such example, the cells are further incubated in a medium comprising a phorbol ester or active derivative thereof according to any example hereof and optionally incubated in or maintained on low-serum medium, and without supplementation of factors normally present in serum, e.g., before, during or following incubation in the presence of one or more agonists of the Akt/(PKB) pathway and/or one or more agonists of the NF-κB pathway. In yet another such example, the cells are further incubated in a medium comprising a retinoid as described according to any example hereof and optionally incubated in or maintained in low-serum medium, and without supplementation of factors normally present in serum, e.g., before, during or following incubation in the presence of one or more agonists of the Akt/(PKB) pathway and/or one or more agonists of the NF-κB pathway. Such additional incubation is for at least about two days i.e., about 48 hours, and not exceeding about ten days i.e., about 240 hours, including for about two days or about three days or about four days or about five days or about six days or about seven days or about eight days or about nine days or about ten days. More preferably, the cells are incubated in low-serum media for a period between about five days and about nine days, including about five days or about six days or about seven days or about eight days or about nine days. Still more preferably, the cells are incubated in low-serum media for a period between about six days and about eight days, including about six days or about seven days or about eight days. In performing such examples, the agonist of the Akt/(PKB) pathway and/or NF-κB pathway may be added to the low-serum medium at any time point in the incubation period of at least about four days and not exceeding about ten days, or a shorter time as indicated herein. The skilled artisan is able to determine appropriate points for addition of one or more agonists to the medium without undue experimentation, and all such routine variations are within the scope of the present invention.

In another example, the cells are further detached by any means known in the art e.g., before, during or following incubation in the presence of one or more agonists of the Akt/(PKB) pathway and/or one or more agonists of the NF-κB pathway. Where optional low-serum medium is employed, is it preferred for such detachment to follow low-serum incubation. As will be apparent from the preceding disclosure, the cells may be detached by incubation in media containing a protease or a ligand of a protease activated receptor (PAR). Alternatively, the cells are detached by incubation in a Ca²⁺-free and Mg⁺-free media containing EDTA. Alternatively, the cells are detached by incubation in a medium containing citric saline.

In another example, the cells are maintained, incubated or cultured under high density conditions e.g., after detachment and in a medium capable of supporting differentiation of progenitor cells or prior to detachment. Such incubation may be before, during or following incubation in the presence of one or more agonists of the Akt/(PKB) pathway and/or one or more agonists of the NF-κB pathway.

In another example, the cells are detached, and detached cells are incubated under high cell density conditions directly in high density plating medium capable of supporting progenitor cells before adherence of the cells to the vessel, e.g., before, during or following incubation in the presence of one or more agonists of the Akt/(PKB) pathway and/or one or more agonists of the NF-κB pathway. In performing such examples, the agonist of the Akt/(PKB) pathway and/or NF-κB pathway may be added to the cells at any time point either during incubation in the culture medium prior to detachment of the cells or during incubation under high density conditions as indicated according to any example hereof. The skilled artisan is able to determine appropriate points for addition of one or more agonists to the medium without undue experimentation, and all such routine variations are within the scope of the present invention.

In a further example, the cells are incubated in low-serum medium and detached and wherein one or more agonists of the Akt/(PKB) pathway and/or one or more agonists of the NF-κB pathway is included in low serum medium and/or in detachment medium. Alternatively, differentiated cells may be treated with one or more agonists of the Akt/(PKB) pathway and/or one or more agonists of the NF-κB pathway before commencing low-serum incubation and/or following detachment.

In a further example, the cells are incubated in low-serum medium, detached and maintained, incubated or cultured under high density conditions wherein one or more agonists of the Akt/(PKB) pathway and/or one or more agonists of the NF-κB pathway is included in low serum medium and/or in detachment medium and/or in high density plating medium. Alternatively, differentiated cells may be treated with one or more agonists of the Akt/(PKB) pathway and/or one or more agonists of the NF-κB pathway before commencing low-serum incubation and/or following high density culture.

It is to be understood that the ordering of the incubation with protease or a ligand of a protease activated receptor (PAR) combined with high density culture, maintenance or incubation, and the incubation in the presence of the agonist(s), and the incubation in the presence of the antagonist(s) with or without extended incubation in low serum medium for about two days to about ten days or shorter periods, is not necessarily essential to the production of cells capable of undergoing subsequent differentiation into a plurality of different cell types. Conveniently, the differentiated cells are incubated in media comprising one or more agonists of the Akt/(PKB) pathway and/or one or more agonists of the NF-κB pathway before incubating the cells in media containing a protease or a ligand of a protease activated receptor (PAR) and before high density incubation conditions in suitable high density plating medium.

It is to also be understood that the ordering of the incubation with a modulator of 5′AMP-activated protein kinase or AMPK combined with protease or a ligand of a protease activated receptor (PAR), and the incubation in the presence of the agonist(s), with or without extended incubation in low serum medium for about two days to about ten days or shorter periods, and with or without high density culture, maintenance or incubation conditions, is not necessarily essential to the production of cells capable of undergoing subsequent differentiation into a plurality of different cell types. Conveniently, the differentiated cells are incubated in media comprising one or more agonists of the Akt/(PKB) pathway and/or one or more agonists of the NF-κB pathway before incubating with a modulator of AMPK and detachment and optionally additional high density culture, maintenance or incubation conditions.

It is also to be understood that the ordering of the incubation with a phorbol ester or active derivative thereof combined with protease or a ligand of a protease activated receptor (PAR), and the incubation in the presence of the agonist(s), with or without extended incubation in low serum medium for about two days to about ten days or shorter periods, and with or without high density culture, maintenance or incubation conditions, is not necessarily essential to the production of cells capable of undergoing subsequent differentiation into a plurality of different cell types. Conveniently, the differentiated cells are incubated in media comprising one or more agonists of the Akt/(PKB) pathway and/or one or more agonists of the NF-κB pathway before incubating with a phorbol ester or active derivative thereof and detachment and optionally additional high density culture, maintenance or incubation conditions.

It is also to be understood that the ordering of the incubation with a retinoid combined with protease or a ligand of a protease activated receptor (PAR), and the incubation in the presence of the agonist(s), with or without extended incubation in low serum medium for about two days to about ten days or shorter periods, and with or without high density incubation, is not necessarily essential to the production of cells capable of undergoing subsequent differentiation into a plurality of different cell types. Conveniently, the differentiated cells are incubated in media comprising one or more agonists of the Akt/(PKB) pathway and/or one or more agonists of the NF-κB pathway before incubating with a retinoid and detachment and optionally additional high density culture, maintenance or incubation conditions.

In accordance with this example, it is preferred for cells produced ex vivo to be formulated for use topically, systemically, or locally as an injectable and/or transplant and/or device, usually by adding necessary buffers. Alternatively, the cells are administered or formulated for use in conjunction with one or more treatments for injuries, allografts or autografts, to enhance wound repair and/or tissue regeneration.

In another example, the present invention provides a method for producing a progenitor cell that is capable of being differentiated into a plurality of different cell types, said method comprising incubating differentiated cells in situ with an amount of an agonist or partial agonist of the Akt/(PKB) pathway and/or NF-kB pathway and for time sufficient to render the cells capable of being differentiated into a plurality of different cell types. This example of the invention is performed on a human or animal subject in situ e.g., without obtaining isolated cells or performing an intermediate cell expansion in culture. In accordance with this example, the agonist or partial agonist of the Akt/(PKB) pathway and/or agonist of the NF-kB pathway is(are) administered directly to a body site in the patient or in the vicinity of a body site in the patient in need of progenitor cells. Such tissue includes without limitation tissue in need of repair such as, for example, an injury, wound, burn, inflamed tissue, degenerated or damaged cardiac tissue, nerve, artery, muscle, bone, cartilage, fat, tendon, ligament, muscle or marrow stroma and combinations thereof.

In another example, the present invention provides a method for producing a progenitor cell that is capable of being differentiated into a plurality of different cell types, said method comprising incubating differentiated cells in situ with an amount of a modulator of 5′AMP-activated protein kinase or AMPK and preferably with an amount of an agonist or partial agonist of the Akt/(PKB) pathway and/or NF-kB pathway and for time sufficient to render the cells capable of being differentiated into a plurality of different cell types. This example of the invention is performed on a human or animal subject in situ e.g., without obtaining isolated cells or performing an intermediate cell expansion in culture. In accordance with this example, a modulator of 5′AMP-activated protein kinase or AMPK and preferably the agonist or partial agonist of the Akt/(PKB) pathway and/or agonist of the NF-kB pathway is(are) administered directly to a body site in the patient or in the vicinity of a body site in the patient in need of progenitor cells. Such tissue includes without limitation tissue in need of repair such as, for example, an injury, wound, burn, inflamed tissue, degenerated or damaged cardiac tissue, nerve, artery, muscle, bone, cartilage, fat, tendon, ligament, muscle or marrow stroma and combinations thereof.

In another example, the present invention provides a method for producing a progenitor cell that is capable of being differentiated into a plurality of different cell types, said method comprising incubating differentiated cells in situ with an amount of a phorbol ester or active derivative thereof and preferably with an amount of an agonist or partial agonist of the Akt/(PKB) pathway and/or NF-kB pathway and for time sufficient to render the cells capable of being differentiated into a plurality of different cell types. This example of the invention is performed on a human or animal subject in situ e.g., without obtaining isolated cells or performing an intermediate cell expansion in culture. In accordance with this example, a phorbol ester or active derivative thereof and preferably the agonist or partial agonist of the Akt/(PKB) pathway and/or agonist of the NF-kB pathway is(are) administered directly to a body site in the patient or in the vicinity of a body site in the patient in need of progenitor cells. Such tissue includes without limitation tissue in need of repair such as, for example, an injury, wound, burn, inflamed tissue, degenerated or damaged cardiac tissue, nerve, artery, muscle, bone, cartilage, fat, tendon, ligament, muscle or marrow stroma and combinations thereof.

In another example, the present invention provides a method for producing a progenitor cell that is capable of being differentiated into a plurality of different cell types, said method comprising incubating differentiated cells in situ with an amount of a retinoid and preferably with an amount of an agonist or partial agonist of the Akt/(PKB) pathway and/or NF-kB pathway and for time sufficient to render the cells capable of being differentiated into a plurality of different cell types. This example of the invention is performed on a human or animal subject in situ e.g., without obtaining isolated cells or performing an intermediate cell expansion in culture. In accordance with this example, a retinoid and preferably the agonist or partial agonist of the Akt/(PKB) pathway and/or agonist of the NF-kB pathway is(are) administered directly to a body site in the patient or in the vicinity of a body site in the patient in need of progenitor cells. Such tissue includes without limitation tissue in need of repair such as, for example, an injury, wound, burn, inflamed tissue, degenerated or damaged cardiac tissue, nerve, artery, muscle, bone, cartilage, fat, tendon, ligament, muscle or marrow stroma and combinations thereof.

Preferred agonists of the Akt/(PKB) pathway suitable for this purpose are described herein and include e.g., interleukin-1 (IL-1), platelet derived growth factor (PGDF-BB), insulin growth factor (IGF-1), transforming growth factor-beta (TGF-β), nerve growth factor (NGF) and carbachol or any active fragment or active chemical group thereof. Preferred agonists of the NF-κB pathway suitable for this purposes are described herein and include e.g., tumor necrosis factor-alpha (TNF-α), interleukin 1 (IL-1), or any active fragment thereof, lysophosphatidic acid (LPA) or lipopolysaccharide (LPS).

In one example, the agonist of the Akt/(PKB) pathway and/or NF-κB pathway and/or a modulator of 5′AMP-activated protein kinase or AMPK and/or a phorbol ester or active derivative thereof and/or a retinoid is/are administered to the site in need thereof for a time and under conditions sufficient to achieve optimum plasticity and/or multipotency or pluripotency of differentiated cells at that site. Such incubation is preferably for a time and under conditions sufficient to induce the Akt/(PKB) pathway and/or NF-κB pathway in the cells or a component thereof that is sufficient to render the cells capable of being differentiated into a plurality of different cell types. The skilled artisan is able to determine appropriate conditions for treatment with one or more agonists without undue experimentation, and all such routine variations are within the scope of the present invention.

In one example, the method further comprises administering a protease or a ligand of a protease activated receptor (PAR) to the site in need of progenitor cells. Without being bound by any theory or mode of action, the administration of a protease or PAR ligand promotes de-differentiation of differentiated cells and/or detachment of integrins from the extracellular matrix, thereby permitting the progenitor cells to enter circulation and regenerate damaged cells and tissues in situ.

It is to be understood that the ordering of the incubation with protease or a ligand of a protease activated receptor (PAR), and the incubation in the presence of the agonist(s) and/or a modulator of 5′AMP-activated protein kinase or AMPK and/or a phorbol ester or active derivative thereof and/or a retinoid is not necessarily essential to the production of cells at the body site capable of undergoing subsequent differentiation into a plurality of different cell types. Conveniently, one or more agonists of the Akt/(PKB) pathway and/or one or more agonists of the NF-κB pathway are administered to the body site before detachment of the cells e.g., by administering a protease or a ligand of a protease activated receptor (PAR). In another example, one or more agonists of the Akt/(PKB) pathway and/or one or more agonists of the NF-κB pathway are administered to the body site before or simultaneously with a modulator of 5′AMP-activated protein kinase or AMPK and/or a phorbol ester or active derivative thereof and/or a retinoid.

Preferred differentiated cells on which the present invention is performed according to any example hereof include e.g., primary cells and immortalized cell lines. It is to be understood that the differentiated cells may also be terminally differentiated cells. The only requirement for such cells in performing this example of the invention is that they do not undergo apoptosis during the period of incubation under high cell density conditions described herein. However, as indicated in the examples, cells that normally undergo apoptosis during exposure to modulator(s) of 5′AMPK and/or exposure to phorbol ester(s) or active derivative(s) thereof and/or exposure to retinoid(s) and optionally during prolonged exposure to high cell density incubation conditions or to serum-free or low-serum media can be used in other examples of the invention employing one or more agonists of the Akt/(PKB) pathway and/or NF-κB pathway to shorten the induction period required to induce plasticity in the starting cells or to enhance plasticity in the starting cells.

Cells of human origin, or potentially cells of porcine origin, are preferred for medical applications. More preferably the cells are derived from a tissue of a subject to whom a downstream product thereof is to be administered i.e., they are autologous. For veterinary or animal improvement purposes, the cells may be derived from any animal species in which they would be compatible when administered. Cells from any commercially-important animal species are contemplated herein e.g., pigs, cattle, horses, sheep, goats, dogs, cats, etc. As with human applications, it is preferred to use autologous cells for such applications to minimize rejection. Exemplary differentiated cells for use in the present invention include skin cells, epidermal cells, fibroblasts, cardiac fibroblasts, keratinocytes, melanocytes, epithelial cells, neural cells such as those derived from the peripheral nervous system (PNS) and central nervous system (CNS), glial cells, Schwann cells, astrocytes, oligodendrocytes, microglial cells, lymphocytes, T cells, B cells, macrophages, monocytes, dendritic cells, Langerhans cells, eosinophils, adipocytes, cardiac muscle cells, osteoclasts, osteoblasts, endocrine cells, β-islet cells, endothelial cells, granulocytes, hair cells, mast cells, myoblasts, Sertoli cells, striated muscle cells, zymogenic cells, oxynitic cells, brush-border cells, goblet cells, hepatocytes, Kupffer cells, stratified squamous cells, pneumocytes, parietal cells, podocytes, synovial cells, serosal cells, pericytes, osteocytes, Purkinje fiber cells, myoepithelial cells, or megakaryocytes.

Chondrocytes can also be used in the present invention for those examples that specifically require high cell density incubation conditions, or incubation with modulator(s) of AMPK, or incubation with phorbol ester(s) or derivative(s) thereof, or incubation with retinoid(s).

In a particularly preferred example, the cells are fibroblasts, preferably of dermal origin.

It will be apparent from the disclosure herein that, in performing the present invention, the differentiated cell is not merely reprogrammed from one developmental pathway to another developmental pathway, but that a progenitor cell is produced that can be stored or otherwise maintained until required for downstream processing e.g., to give rise to different cell types. Without compromising the generality of the present invention, the method of the invention thus produces cells having one or more stem-cell like attributes in so far as they are multipotent, pluripotent or totipotent progenitor cells. For example, the cells produced in accordance with the invention are a novel population of stem cells e.g., having undetectable or low (negligible) levels of at least one and preferably a plurality of the following cell markers as determined by standard cell marker detection assays: CD90, CD117, CD34, CD113, FLK-1, tie-2, Oct 4, GATA-4, NKx2.5, Rex-1, CD105, CD117, CD133, MHC class I receptor and MHC class II receptor. By the term “standard cell marker detection assay” is meant a conventional immunological or molecular assay formatted to detect and optionally quantify one of the foregoing cell markers (i.e., CD90, CD117, CD34 etc.). Examples of such conventional immunological assays include Western blotting, ELISA, and RIA. Preferred antibodies for use in such assays are provided below. See generally, Harlow and Lane in Antibodies: A Laboratory Manual, CSH Publications, N.Y. (1988), for disclosure relating to these and other suitable assays. Particular molecular assays suitable for such use include polymerase chain reaction (PCR) type assays using oligonucleotide primers e.g., as described in WO 92/07075 and/or Sambrook et al. in Molecular Cloning: A Laboratory Manual (2d ed. 1989) and/or Ausubel et al., Current Protocols in Molecular Biology, John Wiley & Sons, New York, 1989.

Accordingly, in a further example, the present invention provides a method for producing a progenitor cell that is capable of being differentiated into a plurality of different cell types, said method comprising preferably incubating differentiated cells in culture media comprising a serum or supplemented with factors normally present in serum, detaching the cells and incubating the cells under high density conditions directly in a high density plating medium before adherence of the cells to thereby render the cells capable of being differentiated into a plurality of different cells types, and maintaining or storing the cells as progenitor cells.

In a further example, the present invention provides a method for producing a progenitor cell that is capable of being differentiated into a plurality of different cell types, said method comprising incubating differentiated cells in media comprising an amount of an agonist or partial agonist of the Akt/(PKB) pathway and/or NF-κB pathway and for time sufficient to render the cells capable of being differentiated into a plurality of different cell types, and maintaining or storing the cells as progenitor cells.

Preferably, the method according to any example hereof further comprises providing the differentiated cells e.g., as an adherent cell culture.

In a further example, the present invention further comprises genetically engineering the progenitor cells to express a protein of interest, such as for example, a macromolecule necessary for cell growth, morphogenesis, differentiation, or tissue building and combinations thereof, and preferably, a bone morphogenic protein, a bone morphogenic-like protein, an epidermal growth factor, a fibroblast growth factor, a platelet derived growth factor, an insulin like growth factor, a transforming growth factor, a vascular endothelial growth factor, Ang-1, P1GF and combinations thereof.

In a further example, the present invention encompasses a cell culture comprising progenitor cells produced by the method disclosed according to any example hereof. Preferably, the cell culture is for treatment of the human or animal body by therapy or prophylaxis.

It will also be apparent from the disclosure herein that the stem cell-like attribute of the progenitor cells produced in accordance with the inventive method confer the ability to produce one or more cells or tissues from them in medical and veterinary applicants and for animal improvement. In this respect, methods for producing such different cell types from a unipotent, multipotent, pluripotent or totipotent progenitor cells are known in the art and/or described herein.

Accordingly, a further example of the present invention provides a process for producing a differentiated cell said process comprising producing a progenitor cell according to any example of the invention hereof and then incubating the progenitor cell for a time and under conditions sufficient to induce differentiation of the progenitor cell into a differentiated cell.

In one such example, the method further comprises incubating the progenitor cell in the presence of at least one mitogen for a time and under conditions sufficient to promote or enhance cell replication and/or cell division of the progenitor cell e.g., by stimulating mitosis, thereby inducing differentiation of the progenitor cell into a differentiated cell. The mitogens falling within the scope of the invention according to any example hereof include but are not limited to Fibroblast growth factors such as FGF-2, amphiregulin, EGF, Sonic hedgehog (Shh), Engrailed 1 (En1) and/or Engrailed 2 (En2), phytohemagglutinin (PHA) including PHA-P, -M, -W, -C, -L and -E+L, pokeweed mitogen (PWM), Concanavalin A (Con-A), lipopolysaccharide (LPS), wheat germ agglutinin (WGA) and soybean agglutinin (SBA), or other genes, proteins and the like.

In another example, the method further comprises incubating the progenitor cell in the presence of at least one morphogen for a time and under conditions sufficient to provide biological signalling suitable for promoting or enhancing cellular specialization and/or cellular differentiation of the progenitor cell, thereby inducing differentiation of the progenitor cell into a differentiated cell. Alternatively, or in addition, the method further comprises incubating the progenitor cell in the presence of at least one compound capable of inducing expression of a morphogen in the cell and/or an agonist or antagonist of a receptor for a morphogen for a time and under conditions sufficient to provide biological signalling suitable for promoting or enhancing cellular specialization and/or cellular differentiation of the progenitor cell, thereby inducing differentiation of the progenitor cell into a differentiated cell. The morphogens falling within the scope of the invention according to any example hereof include but are not limited to retinoic acid and/or homeodomain transcription factors such as Dlx transcription factors eg., Dlx5 and/or fibroblast growth factors such as FGF10, FGF8, FGF4 and/or fibroblast growth factor receptors such as FGFR1, FGFR2 and/or a T-box transcription factors such as TBX4, TBX5 and/or members of the wingless-type MMTV integration site (WNT) signalling factors such as WNT2B, WNT8C, WNT7A, WNT5A, WNT3A and/or Sonic hedgehog (Shh) and/or Bone morphogenetic protein 2 (BMP2) and/or Radical fringe (Rfng) and/or Notch signalling molecules such as Notch, Notch 1, Notch 2, Notch 3, Notch 4 and/or a Notch ligand such as Notch Receptor S1, Jagged 1 (JAG1), Jagged 2 (JAG2) and/or a modulator of Notch signaling such as Lunatic fringe (Lfng), and/or homeodomain proteins such as Lmx1 and/or Ser2 and/or Engrailed 1 (En1) and/or Engrailed 2 (En2) or other genes, proteins and the like.

The progenitor cells may be used to produce any cell type. For example, the differentiated cell produced by the process may be a cardiac tissue cell, skin cell, epidermal cell, keratinocyte, melanocyte, epithelial cell, neural cell, dopaminogenic cell, glial cell, Schwann cell, astrocyte, oligodendrocyte, microglial cell, blood cell, lymphocyte, T cell, B cell, macrophage, monocyte, dendritic cell, lagerhans cell, eosinophil, adipocyte, cardiomyocyte, cardiac muscle cell, cardiac fibroblast, osteoclast, osteoblast, endocrine cell, n-islet cell, insulin secreting cell, endothelial cell, epithelial cell, granulocyte, hair cell, mast cell, myoblast, Sertoli cell, striated muscle cell, zymogenic cell, oxynitic cell, brush-border cell, goblet cell, hepatocyte, Kupffer cell, stratified squamous cell, pneumocyte, parietal cell, podocyte, synovial cell, serosal cell, pericyte, chondrocyte, osteocyte, Purkinje fiber cell, myoepithelial cell, megakaryocyte, etc. In one preferred example, the differentiated cell produced by the method according to any example hereof is a cardiac tissue cell, such as cardiac fibroblast.

As with the progenitor cells of the invention, such differentiated cells can be maintained by one or a combination of strategies including those involving maintenance in vitro. The differentiated cells can be maintained by strategies including those involving maintenance ex vivo and/or in vivo.

Accordingly, in a further example, the present invention encompasses a cell culture comprising differentiated cell produced from a progenitor cell in accordance with the process disclosed according to any example hereof. Preferably, the cell culture is for treatment of the human or animal body by therapy or prophylaxis. In one example, the cell culture is for treatment or prophylaxis of cancer. In another example, the cell culture is for treatment or prophylaxis of tissue or organ damage including but not limited to cardiac injury such as myocardial infraction.

In another example the progenitor cells may used to differentiate into tissues and/or organs. According to this example, the progenitor cells produced by the method of the invention and/or the differentiated cells derived therefrom may be used for regenerating and/or building any tissue or organ. For example, the regenerated or built tissue produced by the process includes, a cardiac tissue, a cardiac muscle tissue, a cardiomyocyte tissue, a cardiac fibroblast tissue, a skin tissue, an epidermal tissue, a keratinocyte tissue, a melanocyte tissue, an epithelial, a dermal dendrocyte tissue, a nervous tissue, a muscle tissue, a connective tissue, a mucosal tissue, an endocrine tissue, an adipose tissue, a galial tissue, a collagen or fibrin tissue, an osseous or bone tissue, an osteocyte tissue, a blood vessel tissue e.g., an endothelial tissue, a lymphoid tissue, an endocrine tissue e.g., a pancreatic endocrine tissue, an islet tissue e.g., p-islet tissue, a chondrocyte tissue, a hepatic tissue, a eosinophil tissue, an osteoblast tissue, an osteoclast tissue, a hair tissue, a bone marrow tissue, a striated muscle tissue, a reproductive tissue, a synovial tissue, etc. For example, the organ regenerated or produced by the process includes heart, artery, trachea, skin, hair, liver, spleen, kidney, muscle, bone, a limb such as a finger and/or toe and/or arm and/or leg, nose, ear, panaceas, lung, lympoid organ, female or male reproductive organ e.g., ovary and/or testis, uterus, vagina, cervix, and fallopian tubes, nerve, blood vessel, small intestine, large intestine, endocrine organ or hormone-secreting gland e.g., pituitary gland, bladder, dental tissues such as teeth, or dentin, etc.

Accordingly in a one example, the present invention further provides a method of regenerating, repairing and/or building a tissue and/or an organ, said method comprising culturing or perfusing the progenitor cells produced according to any example hereof and/or culturing differentiated cells derived from said progenitor cells on or into a biocompatible scaffolding material or matrix. In one example, the scaffold material or matrix, provides the mitogens and/or morphogens and/or biological signalling suitable for promoting or enhancing replication and/or differentiation of the progenitor cells, and/or tissue building repair or regeneration and/or organ building, repair or regeneration. In another example, the scaffold material or matrix provides the structure or outline to a tissue to be repaired, regenerated or built, and/or organ to be repaired, regenerated or built. Such scaffold material or matrix may include for example a non-cellular matrix comprising proteoglycan and/or collagen or other suitable material for tissue building or organ building processes to occur. In one example, the scaffold material or matrix comprises synthetic or semi-synthetic fibers such as Dacron™, Teflon™ or Gore-Tex™. In another example, the scaffold material or matrix comprises a decellularized organ or tissue stripped of its cells by any means known in the art.

Alternatively or in addition, the present invention further provides a method of regenerating, repairing and/or building a tissue and/or an organ, said method comprising providing the progenitor cells an agent selected from the group consisting of: a neuropeptide Y (NPY), a fragment of neuropeptide Y, a variant of neuropeptide Y, a compound capable of inducing expression of a gene encoding a neuropeptide Y protein or fragment or variant thereof, a cell that produces a neuropeptide Y and an agonist or antagonist of a neuropeptide Y receptor, wherein said agent induces regeneration, repair or building of a tissue or organ. In one preferred example, the progenitor cells are provided to a site of injury in a tissue and/or organ to induces regeneration, repair or building of a tissue or organ at the site of injury. In one preferred example, the agent in provided to the progenitor cells at the site of injury

Alternatively or in addition, the present invention provides a method of regenerating, repairing and/or building a tissue and/or an organ, said method comprising providing the progenitor cells an agent selected from the group consisting of: a neuregulin, a fragment of a neuregulin, a compound capable of inducing expression of a neuregulin gene, and an agonist or antagonist of a receptor for neuregulin, wherein said agent induces regeneration, repair or building of a tissue or organ. In one preferred example, the progenitor cells are provided to a site of injury in a tissue and/or organ to induces regeneration, repair or building of a tissue or organ at the site of injury. In one preferred example, the agent in provided to the progenitor cells at the site of injury

Alternatively, or in addition, the present invention provides a method of regenerating, repairing and/or building a tissue and/or an organ, said method comprising providing the progenitor cells an agent selected from the group consisting of: a neurotrophin, a fragment of a neurotrophin, a compound capable of inducing expression of a neurotrophin gene, and/or an agonist or antagonist of a receptor for a neurotrophin, wherein said agent induces regeneration, repair or building of a tissue or organ. In one preferred example, the progenitor cells are provided to a site of injury in a tissue and/or organ to induces regeneration, repair or building of a tissue or organ at the site of injury. In one preferred example, the progenitor cells are provided to a site of injury in a tissue and/or organ to induces regeneration, repair or building of a tissue or organ at the site of injury. In one example, the agent in provided to the progenitor cells at the site of injury. In one non-limiting example, the neurotrophin is nerve growth factor (NGF), or neurotrophic factor 3 (NT-3), or brain derived neurotrophic factor (BDNF), or neurotrophic factor 4 (NT-4), neurotrophic factor 5 (NT-5) or Ciliary Neurotrophic Factor CNTF. In a particularly preferred example, the neurotrophin is NGF.

In one example, tissue and/or organ regeneration, repair or building occurs in vitro externally of the body of an organism, including a human or other mammalian subject in need thereof. In another example, the tissue and/or organ regeneration, repair or building occurs in vivo in an organism, including a human or other mammalian subject in need thereof.

In one example, tissue regeneration or repair or building is used to reduce or eliminate scar tissue.

In one example, the present invention conveniently utilizes a starter cell, i.e., any differentiated primary cell, cell strain, or cell line that is derived and/or obtained from the same tissue type and/or organ type as the tissue and/or organ which is being regenerated, repaired and/or built. For example, skin fibroblasts from a limb or an appendage are used to produce progenitor cells that are subsequently regenerated into a limb or an appendage e.g., a finger, a toe, an arm or a leg. In another example, cardiac fibroblasts such as from a heart or artery are used to produce progenitor cells that are subsequently regenerated into a limb or a cardiovascular organ such as heart or artery.

A further example of the present invention provides for the use of a progenitor cell produced according to any example hereof or a differentiated cell or tissue or organ derived there from in the prophylactic or therapeutic treatment of the human or animal body. In one example, the use of a progenitor cell produced according to any example hereof or a differentiated cell or tissue or organ derived there from is in the treatment or prophylaxis of cancer. In one example, the use of a progenitor cell produced according to any example hereof or a differentiated cell or tissue or organ derived there from is in the treatment or prophylaxis of cardiac or cardiovascular damage or cardiac failure.

In a further example, the present invention provides for the use of a progenitor cell produced according to any example hereof or a differentiated cell or tissue or organ derived there from in the preparation of a cell preparation for the prophylactic or therapeutic treatment of a condition in a subject alleviated by administering stem cells or tissue derived from stem cells to a subject or by grafting stem cells or tissue derived from stem cells into a subject or by transplanting stem cells or tissue derived from stem cells into a subject. In one example, the condition alleviated by administering, grafting or transplanting stem cells or tissue derived from stem cells to a subject is cancer. In another example, the condition alleviated by administering, grafting or transplanting stem cells or tissue derived from stem cells to a subject is cardiac tissue damage or cardiovascular damage.

In a further example, the present invention provides for the use of an isolated, non-culture progenitor cell in the preparation of a medicament for administration to a subject, wherein the non-culture progenitor cell is obtained via a method of the invention according to any example hereof. By “non-culture progenitor cell” is meant a progenitor cell of the present invention produced without cell expansion in vitro and preferably used within about twenty four hours following their preparation by a method described herein according to any example.

In a further example, the present invention provides for the use of an isolated, non-culture progenitor cell in the preparation of a medicament for stimulating or enhancing tissue repair in a subject, wherein the non-culture progenitor cell is obtained via a method of the invention according to any example hereof.

In a further example, the present invention provides for the use of an isolated, non-culture progenitor cell in the preparation of a medicament for stimulating or enhancing tissue formation in a subject, wherein the non-culture progenitor cell is obtained via a method of the invention according to any example hereof.

Preferably, the differentiated cells, tissues or organs are introduced to the human or animal body by grafting means, and it is clearly within the scope of the present invention to provide a graft that includes isolated progenitor cells or differentiated cells or tissues or organs derived there from.

As used herein, the term “graft” shall be taken to mean a cell or tissue or organ preparation that includes an isolated progenitor cell produced in accordance with any example of the invention hereof and/or a differentiated cell, tissue or organ derived in vitro or in vivo from said isolated progenitor cell and, optionally comprising one or more other cells and/or mitogens and/or morphogens and/or a matrix suitable for promoting or enhancing differentiation and/or tissue building, repair or regeneration and/or organ building, repair or regeneration. For example, a “graft” includes tissue or organ that is produced by culturing progenitor cells of the invention and/or differentiated cells derived from said progenitor cells onto a matrix e.g., a non-cellular matrix comprising proteoglycan and/or collagen or other suitable material for tissue building or organ building processes to occur e.g., synthetic or semi synthetic fibers that give structure to a graft, such as Dacron™, Teflon™ or Gore-Tex™. By “graft” is also meant progenitor cells of the invention that have been administered to a recipient and become part of one or more tissues or organs of that recipient. A graft of the invention may also take the form of a tissue preparation or tissue culture preparation in which progenitor cells of the invention have been combined with other cells and/or one or more growth factors and/or one or more mitogens to promote cell proliferation and/or one or more morphogens to promote differentiation and/or cell specialization that produce an intended graft. If desired, the preparation can be combined with synthetic or semi synthetic fibers to give structure to the graft. Fibers such as Dacron™, Teflon or Gore-Tex™ are preferred for certain applications. Sometimes the word “engraftment” will be used to denote intended assimilation of the progenitor cells or derivative differentiated cells tissue or organs into a target tissue, organ or organism, including a human or other mammalian subject. Preferred engraftment involves neural tissue, cardiovascular tissue, cardiac tissue, splenic tissue, pancreatic tissue, etc.

In using the cells of the invention for medical applications or veterinary applications, or in animal improvement, immunological relationship between a donor of the differentiated cells used to produce the progenitor cells, and the recipient of the progenitor cells or a cell or tissue or organ derived from the progenitor cells, can be allogenic, autologous, or xenogeneic as needed. In one example, the donor and recipient will be genetically identical and usually will be the same individual (syngeneic). In this instance, the graft will be syngeneic with respect to the donor and recipient. In one example, the progenitor cells and/or graft will be immune tolerated in the recipient subject.

A further example of the present invention provides a method for preventing, treating or reducing the severity of a disease or disorder in a human or animal subject said method comprising administering to the human or animal subject in need of treatment at least one isolated progenitor cell or graft or a combination thereof. Preferably, the administration is sufficient to prevent, treat or reduce the severity of the disease or disorder in the human or animal subject.

In one example, the method further includes incubating the cells or graft in the human or animal subject for at least about a week, preferably between from about two to eight weeks. It will be apparent to those working in the field that the incubation period is flexible and can be extended or shorten to address a particular indication or with respect to the health or age of the individual in need of treatment. Typical amounts of progenitor cells to use will depend on these and other recognized parameters including the disease to be treated and the speed of recovery needed. However for most applications between from about 1×10³ to about 1×10⁷ progenitor cells per grafting site will suffice, typically about 1×10⁵ of such cells. Cells may be administered by any acceptable route including suspending the cells in saline and administering same with a needle, stent, catheter or like device. In examples in which myocardial ischemia or an infarct is to be addressed, the administration will be a bolus injection near or directly into the desired site.

In another example, the method further includes administering to the human or animal subject in need of treatment at least one growth factor and/or at least one mitogen and/or at least one morphogen and/or a functional fragment thereof to promote tissue regeneration and/or cellular proliferation. Alternatively, or in addition, the method can include administering to the mammal at least one nucleic acid encoding at least one growth factor and/or at least one mitogen and/or at least one morphogen and/or a functional fragment thereof. For example, methods for administering such nucleic acids to mammals have been disclosed by U.S. Pat. No. 5,980,887 and WO 99/45775.

In yet another example, the method further includes administering to the human or animal subject one or more other progenitor cells.

Further provided by the invention is a pharmaceutical composition for preventing, treating or reducing the severity of a disease or disorder, said composition comprises a population of progenitor cells or graft produced according to any example hereof and a pharmaceutically acceptable carrier. Optionally, the composition comprises directions for preparing, maintaining and/or using the progenitor cells or graft, including any cell culture, tissue or organ. In one example, the product further includes at least one growth factor and/or mitogen and/or morphogen and/or a functional fragment thereof. In another example, the product further comprises at least one nucleic acid encoding a growth factor, mitogen, morphogen and/or a functional fragment thereof.

Further provided by the invention is a kit for building, repairing or regenerating a tissue or an organ, said kit comprises a population of progenitor cells produced according to any example hereof and a scaffold or matrix for culturing the progenitor cells or differentiated cells produced from said progenitor cells. Optionally, the composition comprises directions for preparing, maintaining and/or using the progenitor cells or graft, including any cell culture, tissue or organ. In one example, the product further includes at least one growth factor and/or at least one mitogen and/or at least one morphogen and/or a functional fragment thereof. In another example, the product further comprises at least one nucleic acid encoding a growth factor, and/or a mitogen, and/or a morphogen and/or a functional fragment thereof.

Further provided by the invention is an isolated differentiated cells derived from progenitor cells produced according to any example hereof. Also provided by the invention is a scaffold or martix comprising progenitor cells or one or more populations of differentiated cells derived from the progenitor cells as described according to any example hereof. Further provided by the invention is any tissue or any organ derived in vitro or in vivo from isolated progenitor cells produced according to any example hereof or any tissue or any organ derived in vitro or in vivo from differentiated cells derived from the progenitor cells as described according to any example hereof.

In further examples, the invention as described according to any example hereof may consist essentially of a stated step or element or integer or group of steps or elements or integers.

In further examples, the invention as described according to any example hereof may consist of a stated step or element or integer or group of steps or elements or integers.

Unless the context requires otherwise or specifically stated to the contrary, integers, steps, or elements of the invention recited herein as singular integers, steps or elements clearly encompass both singular and plural forms of the recited integers, steps or elements.

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, developmental biology, mammalian cell culture, recombinant DNA technology, histochemistry and immunohistochemistry and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Methods In Enzymology (S. Colowick and N. Kaplan, eds., Academic Press, Inc.), whole of series;

In further examples, the subject matter described and/or claimed in the applicant's earlier-published International application WO 2009/097657 (Aug. 13 2009) is disclaimed.

### DETAILED DESCRIPTION OF THE PREFERRED EMBODIMENTS

### 1. Cell Types-Starting Material

The present invention contemplates that any differentiated animal cell type including terminally differentiated animal cells may be used as starting cells for the method of the invention. For example, the starting cells are primary cells, a cell strain, or a cell line.

By “differentiated cell type” is meant a differentiated animal cell type that expresses defined specialized properties that are characteristic of that cell type. These defined specialized properties are passed onto daughter cells if the differentiated cell type undergoes cellular division. The differentiated cell type may be a cell type that is not actively proliferating. Methods for determining the expressed specialized properties will be apparent to the skilled artisan and/or described herein.

In one example, the starting cells are readily available in substantial quantities. The starting cells may be derived from any animal and preferably from a mature adult animal. The type of animal preferably includes but is not limited to humans, and includes any animal species such as: other primate such as ape, chimpanzee, gorilla, monkey, or orangutan, horse, cow, goat, sheep, pig, dog, cat, bird, fish, rabbit, rodent, such as a mouse or rat.

The present invention also contemplates that the starting cells may be expanded in cell culture prior to use. Methods for expanding the starting cells in culture will be apparent to the skilled artisan and/or described herein.

### 1.1 Primary Cell Cultures, Cell Strains, and Cell-Lines

Methods for obtaining primary cultures of differentiated starting cells and others are well known in the art, and usually include obtaining the tissue from a biopsy, amputated limb, secretion, excretion, or other source. The tissue may be derived from any part of the body that is readily available including, but not limited to organs such as skin, bone, gut, pancreas, thymus, spleen, blood, bone marrow, spine, any nervous tissue, or any cardiac or cardiovascular tissue e.g., heart or artery.

In a preferred example, the tissue is derived from an adult donor. In a further preferred example the tissue is derived from a patient, since this facilitates autologous transplants and thus reduces the likelihood of adverse immunogenic reactions in the patient. In another preferred example, the tissue is derived from a damaged and/or amputated organ e.g., limb or appendage which is in need of regeneration, repair or replacement in the patient.

The tissue sample comprising the desired differentiated starting cells may also contain connective tissue, for example a skin biopsy. As a non-limiting example, in order to isolate human dermal fibroblasts derived from a mammal, preferably a human, a skin biopsy is obtained which is then minced or otherwise cut into smaller pieces or treated to release the differentiated cell. For instance, and without limiting the invention to any particular method of obtaining a cell to be used in the methods described herein, the tissue is often treated with a collagenase or other protease in order to disassociate the cells from the tissue aggregate. These cells are then placed in a tissue culture flask, or dish, along with a nutrient tissue culture media and propagated at a suitable temperature and a suitable CO₂ saturation. The suitable temperature is often from about 35° C. to about 37° C., and the suitable CO₂ saturation is often about 5-10% in air.

Optionally, the tissue sample may also be a suspension containing cells, or comprising a liquid such as a blood sample, or an aspirate such as fluid obtained from the spinal column, or from bone marrow. Samples obtained in suspension and/or liquid form are further processed by centrifugation, or separation, and culture techniques. Blood cells and lymphocytes are often obtained from whole blood treated with heparin or another anti-coagulant. The blood is centrifuged on a gradient, such as a Ficoll gradient, and the lymphocytes and other blood cells form a distinct layer often referred to as the “buffy coat”. Primary lymphocytes procured by this method can be further separated by their adherence to glass or plastic (monocytes and macrophages adhere, other lymphocytes, in general, do not adhere). Methods for obtaining and culturing both solid tissue and blood cells from a human are well known in the art and are described in, for example, Freshney (2000, Culture of Animal Cells: A Manual of Basic Techniques, 4th Edition, Wiley-Liss, New York, N.Y.).

As a further non-limiting example, peripheral nerve tissue can be obtained using surgical procedures such as nerve biopsies, amputated limbs, and from organ donors and by any other methods well known in the art or to be developed. Potential sources of peripheral nerve include the sciatic nerve, cauda equina, sural nerve of the ankle, the saphenous nerve, the sciatic nerve, or the brachial or antebrachial nerve of the upper limb.

A preferred amount for the starting nerve tissue is between about 10 milligrams to about 10 grams, preferably between about 100 milligrams to about 1-2 gram. Primary human Schwann cells can be isolated and cultured using the methods detailed elsewhere in this invention or methods known in the art. Other methods for the isolation and culture of Schwann cells and other neural cells are well known in the art, and can readily be employed by the skilled artisan, including methods to be developed in the future. The present invention is in no way limited to these or any other methods, of obtaining a cell of interest.

The skilled artisan would appreciate, based upon the disclosure provided herein, that the particular method for obtaining a differentiated starting cell of interest is not limited in any way, but encompasses methods for isolating a cell of interest well known in the art or to be developed in the future.

In one example, the differentiated starting cells employed in the present invention include, but are not limited to skin cells, epidermal cells such as fibroblasts, keratinocytes, and melanocytes, and epithelial cells and the like, cardiac tissue or cardio vascular tissue cells such as cardiac muscle cells, cardiac fibroblasts, cardiomyocytes, neural cells such as those derived from the peripheral nervous system (PNS) and central nervous system (CNS) including, but not limited to, glial cells, such as, e.g., Schwann cells, astrocytes, oligodendrocytes, microglial cells, and blood cells, such as lymphocytes, including T cells and B cells, macrophages, monocytes, dendritic cells, Lagerhans cells, eosinophils, and the like, adipocytes, osteoclasts, osteoblasts, endocrine cells, islet cells of the pancreas, endothelial cells, epithelial cells, granulocytes, hair cells, mast cells, myoblasts, Sertoli cells, striated muscle cells, zymogenic cells, oxynitic cells, brush-border cells, goblet cells, hepatocytes, Kupffer cells, stratified squamous cells, pneumocytes, parietal cells, podocytes, synovial cells, such as synovial fibroblasts, serosal cells, pericytes, chondrocytes, osteocytes, Purkinje fiber cells, myoepithelial cells, megakaryocytes, and the like.

The present invention further includes starting cells of primary cells from any of the aforementioned sources that may be purchased from any commercial source including PromoCell® (Banksia Scientific Company, QLD).

The present invention further includes starting cells of primary cells obtained from or present in the human or animal body.

The present invention further includes starting cells of primary strains or cells lines established in culture, or to be established in culture in the future that may be purchased from any commercial source including American Type Culture Collection (Rockville, Md.).

By “primary strain” shall be taken to indicate any cell type derived as described by any example herein that is established in culture, and that expresses defined specialized properties that are passed onto daughter cells during cellular division, and have a limited life span in culture. Methods for determining the expressed specialized properties will be apparent to the skilled artisan and/or described herein.

By “cell line” shall be taken to indicate any cell type derived as described by any example herein that is established in culture, and that expresses defined specialized properties that are passed onto daughter cells during cellular division, and have an indefinite life span in culture. Methods for determining the expressed specialized properties will be apparent to the skilled artisan and/or described herein.

Preferably, the primary strain, or cell line used as starting material has specialized properties that define the cell type, and such defined cell types include, but are not limited to: skin cells, epidermal cells, such as fibroblasts, keratinocytes, and melanocytes, and epithelial cells, and cardiac tissue or cardio vascular tissue cells such as cardiac muscle cells, cardiac fibroblasts, cardiomyocytes, and neural cells such as those derived from the peripheral nervous system (PNS) and central nervous system (CNS) including, but not limited to, glial cells, such as, e.g., Schwann cells, astrocytes, oligodendrocytes, microglial cells, and blood cells, such as lymphocytes, including T cells and B cells, macrophages, monocytes, dendritic cells, Lagerhans cells, eosinophils, and the like, adipocytes, osteoclasts, osteoblasts, endocrine cells, β-islet cells of the pancreas, endothelial cells, epithelial cells, granulocytes, hair cells, mast cells, myoblasts, Sertoli cells, striated muscle cells, zymogenic cells, oxynitic cells, brush-border cells, goblet cells, hepatocytes, Kupffer cells, stratified squamous cells, pneumocytes, parietal cells, podocytes, synovial cells, such as synovial fibroblasts, serosal cells, pericytes, chondrocytes, osteocytes, Purkinje fiber cells, myoepithelial cells, megakaryocytes, and the like.

In one example the present invention utilizes a starter cell that is not sensitive to high cell density culture conditions used by the method of the invention. For example, such a starter cell is not sensitive to incubation of cells at a starting density of detached cells of about 1500 cells/mm² plating surface area to about 200,000 cells/mm² plating surface area or greater, including about 1,850 cells/mm² surface area of the culture vessel or greater, or about 2,220 cells/mm² surface area of the culture vessel or greater, or about 2,590 cells/mm² surface area of the culture vessel or greater, or about 2,960 cells/mm² surface area of the culture vessel or greater, or about 2,220 cells/mm² surface area of the culture vessel or greater, or about 3,330 cells/mm² surface area of the culture vessel or greater, or about 3,703 cells/mm² surface area of the culture vessel surface area of the culture vessel or greater, or about 7,407 cells/mm² surface area of the culture vessel surface area of the culture vessel or greater.

By “starter cell” is taken to mean any differentiated primary cell, cell strain, or cell line as derived and/or obtained by any example described herein.

By “not sensitive to high cell density culture conditions” is taken to mean does not undergo cell death and/or has activated one or more pro-survival pathway(s). For example, cell death is the result of the induction or outcome of any cellular process that includes but is not limited to necrosis, apoptosis or programmed cell death.

By “pro-survival pathway” is taken to mean any pathway that overcomes the induction of one or more cellular processes that result in cell death.

In another example, the present invention utilizes a starter cell that is not sensitive to high cell density culture conditions, and that is induced in one or more pro-survival pathway(s) such that the incubation at high cell density condition does not affect survival of the cell. The present invention contemplates the induction of any cellular pro-survival pathway known in the art, or that may become known in the future such that it may be induced to survival of the cell under high cell density conditions.

In another example, the present invention utilizes a starter cell that is sensitive to high cell density culture conditions, and induced in one or more pro-survival pathway(s) such that the incubation time in high cell density conditions can be reduced and/or the survival of the cell in high density conditions is enhanced. The present invention contemplates the induction of any cellular pro-survival pathway known in the art, or that may become known in the future such that it may be induced to reduce the culture time in high cell density culture conditions, and/or enhance survival of the cell under such conditions. By “sensitive to high density culture conditions” is taken to mean undergoes cell death and/or has not activated one or more pro-survival pathway(s). For example, cell death is the result of the induction or outcome of any cellular process that includes but is not limited to necrosis, apoptosis or programmed cell death.

In one example, survival of the cells in high density culture conditions as described in any example herein is enhanced by activating the Akt/(PKB) pathway, also referred to as protein kinase B (PKB). In another example, survival of the cells in high density culture conditions as described in any example herein is enhanced by activating the NF-κB pathway.

In one example the present invention utilizes a starter cell that is not sensitive to low-serum culture conditions. For example, such a starter cell is not sensitive to culturing in low serum for the period the cell is required to be maintained in low serum conditions. By “not sensitive to low-serum culture conditions” is taken to mean does not undergo cell death and/or has activated one or more pro-survival pathway(s). For example, cell death is the result of the induction or outcome of any cellular process that includes but is not limited to necrosis, apoptosis or programmed cell death.

In another example, the present invention utilizes a starter cell that is not sensitive to low-serum culture conditions, and that is induced in one or more pro-survival pathway(s) such that the incubation time in low-serum can be reduced. The present invention contemplates the induction of any cellular pro-survival pathway known in the art, or that may become known in the future such that it may be induced to reduce the culture time in low serum.

In another example, the present invention utilizes a starter cell that is sensitive to low-serum culture conditions, and induced in one or more pro-survival pathway(s) such that the incubation time in low-serum can be reduced. The present invention contemplates the induction of any cellular pro-survival pathway known in the art, or that may become known in the future such that it may be induced to reduce the culture time in low serum. By “sensitive to low-serum culture conditions” is taken to mean undergoes cell death and/or has not activated one or more pro-survival pathway(s). For example, cell death is the result of the induction or outcome of any cellular process that includes but is not limited to necrosis, apoptosis or programmed cell death.

In one example, the culture time in low-serum as described in any example herein is reduced by any time less than 7 days by activating the Akt/(PKB) pathway, also referred to as protein kinase B (PKB). In another example, the culture time in low-serum as described in any example herein is reduced by activating the NF-κB pathway.

In one example, the present invention utilizes a starter cell that is not sensitive to incubation with a modulator of 5′AMP-activated protein kinase or AMPK used by the method of the invention. For example, such a starter cell is not sensitive to culturing with 5′AMP-activated protein kinase or AMPK for the period the cell is required to be cultured, maintained or incubated in the presence of the modulator.

By “not sensitive to incubation with a modulator of 5′AMP-activated protein kinase or AMPK” or “not sensitive to modulation of 5′AMP-activated protein kinase or AMPK” is taken to mean that the cell does not undergo cell death and/or has activated one or more pro-survival pathway(s). For example, cell death is the result of the induction or outcome of any cellular process that includes but is not limited to necrosis, apoptosis or programmed cell death.

In another example, the present invention utilizes a starter cell that is not sensitive to incubation with a modulator of AMPK and/or to modulation of AMPK in the cell, and that is induced in one or more pro-survival pathway(s) such that incubation time with a modulator of AMPK or modulation time of AMPK in the cell can be reduced and/or does not significantly affect viability of the cell. The present invention contemplates the induction of any cellular pro-survival pathway known in the art, or that may become known in the future such that it may be induced to enhance viability of the cell in response to modulation of AMPK activity and/or to reduce the culture time with a modulator of AMPK.

In another example, the present invention utilizes a starter cell that is sensitive to incubation with a modulator of AMPK and/or to modulation of AMPK in the cell, and that is induced in one or more pro-survival pathway(s) such that incubation time with a modulator of AMPK or modulation time of AMPK in the cell can be reduced and/or does not significantly affect viability of the cell. The present invention contemplates the induction of any cellular pro-survival pathway known in the art, or that may become known in the future such that it may be induced to enhance viability of the cell in response to modulation of AMPK activity and/or to reduce the culture time with a modulator of AMPK. By “sensitive to incubation with a modulator of 5′AMP-activated protein kinase or AMPK and/or to modulation of 5′AMP-activated protein kinase or AMPK in the cell” is taken to mean undergoes cell death and/or has not activated one or more pro-survival pathway(s). For example, cell death is the result of the induction or outcome of any cellular process that includes but is not limited to necrosis, apoptosis or programmed cell death.

In one example, survival of the cell following incubation with a modulator of 5′AMP-activated protein kinase or AMPK and/or to modulation of 5′AMP-activated protein kinase or AMPK in the cell as described in any example herein is enhanced by activating the Akt/(PKB) pathway, also referred to as protein kinase B (PKB). In another example, survival of the cell following incubation with a modulator of 5AMP-activated protein kinase or AMPK and/or to modulation of 5AMP-activated protein kinase or AMPK in the cell as described in any example herein is enhanced by activating the NF-κB pathway.

In one example, the present invention utilizes a starter cell that is not sensitive to incubation with a phorbol ester or active derivative thereof. For example, such a starter cell is not sensitive to culturing with a phorbol ester or active derivative thereof for the period the cell is required to be maintained in the presence of the phorbol ester or active derivative thereof. By “not sensitive to incubation with a phorbol ester or active derivative thereof is taken to mean that the cell does not undergo cell death and/or has activated one or more pro-survival pathway(s). For example, cell death is the result of the induction or outcome of any cellular process that includes but is not limited to necrosis, apoptosis or programmed cell death.

In another example, the present invention utilizes a starter cell that is not sensitive to incubation with a phorbol ester or active derivative thereof, and that is induced in one or more pro-survival pathway(s) such that incubation time with a phorbol ester or active derivative thereof can be reduced and/or does not significantly affect viability of the cell. The present invention contemplates the induction of any cellular pro-survival pathway known in the art, or that may become known in the future such that it may be induced to enhance survival of the cell and/or to reduce the culture time in presence of a phorbol ester or an active derivative thereof.

In another example, the present invention utilizes a starter cell that is sensitive to incubation with a phorbol ester or active derivative thereof, and that is induced in one or more pro-survival pathway(s) such that such that incubation time with a phorbol ester or active derivative thereof can be reduced and/or does not significantly affect viability of the cell. The present invention contemplates the induction of any cellular pro-survival pathway known in the art, or that may become known in the future such that it may be induced to enhance survival of the cell and/or to reduce the culture time in presence of a phorbol ester or an active derivative thereof. By “sensitive to incubation with a phorbol ester or active derivative thereof” is taken to mean undergoes cell death and/or has not activated one or more pro-survival pathway(s). For example, cell death is the result of the induction or outcome of any cellular process that includes but is not limited to necrosis, apoptosis or programmed cell death.

In one example, survival of the cell following incubation with a phorbol ester or active derivative thereof as described in any example herein is enhanced by activating the Akt/(PKB) pathway, also referred to as protein kinase B (PKB). In another example, survival of the cell following incubation with a phorbol ester or active derivative thereof as described in any example herein is enhanced by activating the NF-κB pathway.

In one example, the present invention utilizes a starter cell that is not sensitive to incubation with a retinoid. For example, such a starter cell is not sensitive to culturing with a retinoid for the period the cell is required to be maintained in the presence of the retinoid. By “not sensitive to incubation with a retinoid is taken to mean that the cell does not undergo cell death and/or has activated one or more pro-survival pathway(s). For example, cell death is the result of the induction or outcome of any cellular process that includes but is not limited to necrosis, apoptosis or programmed cell death.

In another example, the present invention utilizes a starter cell that is not sensitive to incubation with a retinoid, and that is induced in one or more pro-survival pathway(s) such that incubation time with a retinoid can be reduced and/or does not significantly affect viability of the cell. The present invention contemplates the induction of any cellular pro-survival pathway known in the art, or that may become known in the future such that it may be induced to enhance survival of the cell and/or to reduce the culture time in presence of a retinoid.

In another example, the present invention utilizes a starter cell that is sensitive to incubation with a retinoid, and that is induced in one or more pro-survival pathway(s) such that such that incubation time with a retinoid can be reduced and/or does not significantly affect viability of the cell. The present invention contemplates the induction of any cellular pro-survival pathway known in the art, or that may become known in the future such that it may be induced to enhance survival of the cell and/or to reduce the culture time in presence of a retinoid. By “sensitive to incubation with a retinoid” is taken to mean undergoes cell death and/or has not activated one or more pro-survival pathway(s). For example, cell death is the result of the induction or outcome of any cellular process that includes but is not limited to necrosis, apoptosis or programmed cell death.

In one example, survival of the cell following incubation with a retinoid as described in any example herein is enhanced by activating the Akt/(PKB) pathway, also referred to as protein kinase B (PKB). In another example, survival of the cell following incubation with a retinoid as described in any example herein is enhanced by activating the NF-κB pathway.

### 1.2. Modulation of 5′AMP-Activated Protein Kinase or AMPK

Without being bound by any theory or mode of action, it is thought that 5′AMP-activated protein kinase or AMPK plays a key role in regulation of carbohydrate and fat metabolism, serving as “a metabolic master switch” in response to alterations in cellular energy charge. (Winder et al, Am J Physiol, 2777: E1-10, 1999, herein incorporated by reference in its entirety; Winder, J Appl Physiol, 91:1017-1028, 2001, herein incorporated by reference in its entirety). For example, 5′AMP-activated protein kinase or AMPK phosphorylates numerous target proteins at serine residues in the context of a characteristic sequence recognition motif, and the resulting phosphorylation, in turn, may increase or decrease the rate of the metabolic pathway in which the protein target plays a regulatory role.

The method of modulating 5′AMP-activated protein kinase or AMPK in a starter cell may comprise contacting the starter cell with any one or more factors that modulate(s) the 5′AMP-activated protein kinase or AMPK phosphorylation and/or activity, thereby increases or decreasing the rate at which AMPK phosphorylates any one of its numerous protein targets. The term is not limited by the mechanism underlying how the rate at which AMPK phosphorylates any one of its numerous protein targets is increased or decreased. The potential mechanisms through which such a compound may act include, but are not limited to, allosteric mechanisms that affect, directly or indirectly, AMPK activity, as well as mechanisms that act, directly or indirectly, to promote the phosphorylation of the AMPK catalytic subunit catalyzed by upstream kinase e.g., AMPK kinase (AMPKK) or calmodulin-dependent protein kinase kinase β (CaMKKβ).

In one example, the modulator is an inducer that initiates and/or enhances activation of the 5′AMP-activated protein kinase or AMPK in a starter cell. Such an inducer is also referred to as 5′AMP-activated protein kinase or AMPK enhancer or an 5′AMP-activated protein kinase or AMPK agonist. For example, the inducer is a peptide, a polypeptide, a chemical, a nucleic acid, an antibody, an antibody fragment or a small molecule, or any insult that induces cellular metabolic stress such as, but not limited to, glucose starvation, increased cellular AMP [5′-adenosine monophosphate] concentrations, hypoxia, ischemia or UV irradiation.

The present invention contemplates any inducer of the 5′AMP-activated protein kinase or AMPK known in the art or to be developed in the future. Preferably, the inducer of 5′AMP-activated protein kinase or AMPK includes, but is not limited to factors such as AICAR [5-aminoimidazole-4-carboxamide-1-β-4-ribofuranoside], a phosphorylated AICAR-riboside or ZMP [5-aminoimidazole-4-carboxamide-ribotide], Metformin (Glucophage) [1,1-dimethylbiguanide], an appetite-stimulating hormone ghrelin/obestatin prepropeptide (GHRL), 3PG [3-Phosphoglyceric acid], Thrombin, extracellular AMP [5′-adenosine monophosphate], long chain fatty acyl analogs such as acyl-CoA thioester.

A method for inducing 5′AMP-activated protein kinase or AMPK with AICAR includes as described in Mukherjee at al., Mol. Cancer, 7: 37 (2008) or Lubna Al-Khalili at al., Am J Physiol Endocrinol Metab 287:553-557 (2004) or Gaidhu M P et al., J Biol Chem. 8; 281(36):25956-64 (2006) or any references as described therein. A method for inducing 5′AMP-activated protein kinase or AMPK with ZMP includes as described in Gadalla et al., J. Neurochem. 88:1272-1282 (2004) or any references as described therein. A method for inducing 5′AMP-activated protein kinase or AMPK with Metformin includes as described in Lee G. D. Frye et al., J Bio Chem 277(28):25226-25232 (2002) or Leclerc et al., Am J Physiol Endocrinol Metab 286: E1023-E1031(2004) or Zhou G et al., J Clin Invest 108:1167-1174 (2001) any references as described therein. A method for inducing 5′AMP-activated protein kinase or AMPK with ghrelin includes as described in Murata et al., J Bio Chem. Vol. 277(7):5667-5674 (2002) or any references as described therein. A method for inducing 5′AMP-activated protein kinase or AMPK with thrombin includes as described in Stahmann et al., Mol. Cell. Biol. 26(16): 5933-5945 (2006) or any references as described therein. A method for inducing 5′AMP-activated protein kinase or AMPK with extracellular AMP [5′-adenosine monophosphate] includes as described in Aymerich et al., Journal of Cell Science 119(8): 1612-1621 (2006) or any references as described therein. A method for inducing 5′AMP-activated protein kinase or AMPK with long chain fatty acyl analogs such as acyl-CoA thioester includes as described in Za'tara et al., Biochemical Pharmacology 76: 1263-1275 (2008) or any references as described therein.

In another example, the modulator is an inhibitor that inhibits and/or suppresses activation and/or function of the 5′AMP-activated protein kinase or AMPK in a starter cell. Such an inhibitor is also referred to as 5′AMP-activated protein kinase or AMPK suppressor or an 5′AMP-activated protein kinase or AMPK antagonist. For example, the inhibitor is a peptide, a polypeptide, a chemical, a nucleic acid, an antibody, an antibody fragment or a small molecule, or any insult that induces cellular metabolic stress such as, but not limited to, increase in glucose concentration or decreased cellular AMP [5′-adenosine monophosphate], high cellular ATP concentrations or high glycogen content or physiological concentrations of phosphocreatine.

The present invention contemplates any inhibitor of the 5′AMP-activated protein kinase or AMPK known in the art or to be developed in the future. Preferably, the inhibitor of 5′AMP-activated protein kinase or AMPK includes, but is not limited to factors such as Compound C or Dorsomorphin (6-[4-(2-Piperidin-1-yl-ethoxy)-phenyl)]-3-pyridin-4-yl-pyrrazolo[1,5-a]-pyrimidine), Metformin (Glucophage) [1,1-dimethylbiguanide], an appetite-stimulating hormone ghrelin/obestatin prepropeptide (GHRL), glycogen, long-chain acyl-CoA esters (LCACEs) or PPARα agonist (aA) and PPARα/γ dual agonist or phosphocreatine.

A method for inhibiting 5′AMP-activated protein kinase or AMPK with Compound C includes as described in Mukherjee at al., Mol. Cancer, 7: 37 (2008) or any references as described therein. A method for inhibiting 5′AMP-activated protein kinase or AMPK with Metformin includes as described in Chau-Van et al., Endocrinology. 148(2):507-511 (2006) or any references as described therein. A method for inhibiting 5′AMP-activated protein kinase or AMPK with ghrelin includes as described in Leontiou et al., Endocrine Abstracts 13:P205 (2007) or any references as described therein.

In another preferred example, the modulator of 5′AMP-activated protein kinase or AMPK includes activating a receptor that modulates the 5′AMP-activated protein kinase or AMPK by contacting the receptor of a starter cell with a peptide, a polypeptide, a chemical, a nucleic acid, an antibody, an antibody fragment or a small molecule, such that the receptor activation modulates the 5′AMP-activated protein kinase or AMPK activation and/or function.

Preferably, the receptor is an adenosine receptor such as A₁ adenosine receptor, A_{2A} adenosine receptor or the A_{2B} adenosine receptor, or the neural receptor GABA_{B} or the adipose tissue receptors such as adiponectin receptors, or the Vascular endothelial growth factor receptor 2 (VEGFR-2), or the Fibroblast growth factor receptor (FGFR), or the Gq-coupled receptors, or the growth hormone secretagogue receptor (GHS-R).

It will be understood that modulators of 5′AMP-activated protein kinase or AMPK falling with the scope of the invention exclude activators of the Akt/(PKB) pathway including but not limited to e.g., interleukin-1 (IL-1), platelet derived growth factor (PGDF-BB), insulin growth factor (IGF-1), transforming growth factor-beta (TGF-β), nerve growth factor (NGF) and carbachol or any active fragment or active chemical group thereof. It is also understood that modulators of 5′AMP-activated protein kinase or AMPK falling with the scope of the invention exclude activators of the NF-κB pathway including but not limited to e.g., tumor necrosis factor-alpha (TNF-α), interleukin 1 (IL-1), or any active fragment thereof, lysophosphatidic acid (LPA) or lipopolysaccharide (LPS).

A method to measure the phosphorylation or activation of 5′AMP-activated protein kinase or AMPK includes any method that measures the activity of 5′AMP-activated protein kinase or AMPK, or any known intracellular intermediate of the 5′AMP-activated protein kinase or AMPK activation, as described in Al-Khalili et al., AM J Physiol Endocrinol Metab 287: E553-E557 (2004), or any reference described therein. For example, the phosphorylation of AMPK at Thr-172 may be used as a marker of the activation of the 5′AMP-activated protein kinase or AMPK. An immunoblot method to measure AMPK phosphorylation is described in Al-Khalili et al. Briefly, after incubation with factor(s) that induce AMPK activation, cells are harvested and laysed on ice and transferred onto nitrocellulose membranes for immunoblotting. Membranes and incubated with an antibody that detects phosphorylation of AMPK at Thr-172 (Cell Signalling Technology, Beverly Mass.). Enhanced chemoilluminescence is used to visualize protein bands that may be quantitated using a phosphoimager. Values may be expressed relative to control cells not incubated with factor(s) that induce AMPK activation. Alternatively or in addition thereto, AMPK activity is studied with kinase assays using phosphorylation specific antibodies for the AMPK al and/or AMPK a2 units in the cell lysates as described in Fryer et al., J Bio Chem 277(28):2526-25232. Briefly, cells are incubated with factor(s) that induce AMPK activation. Following this incubation, cells are rinsed in phosphate-buffered saline (5 mM NaH2PO4, pH 7.4, 150 mM NaCl) and lysed e.g., by addition of 0.25 ml of 50 mM Tris/HCl, pH 7.5, 50 mM NaF, 5 mM sodium pyrophosphate, 1 mM dithiothreitol, 10% (v/v) glycerol, 1% (v/v) Triton X-100. Insoluble material is removed by centrifugation, and protein concentration determined e.g., using the Bradford reagent. AMPK 1-containing complexes are immunoprecipitated by incubation with an anti-1 antibody prebound to protein G-Sepharose for 2 h at 4° C. AMPK 2-containing complexes are recovered from the supernatant of this incubation by immunoprecipitation with an anti-2 antibody prebound to protein G-Sepharose. For total AMPK activity, lysates are immunoprecipitated using a pan-antibody prebound to protein A-Sepharose. AMPK activity present in the immune complexes is measured by phosphorylation of the synthetic substrate HMRSAMSGLHLVKRR (SAMS) peptide in the presence of 5 mM MgCl₂, 0.2 mM [γ-³²P ATP] and 0.2 mM AMP as described in Stahmann et al., Mol. Cell. Biol. 26(16): 5933-5945 (2006) or Davies et al., Eur. J. Biochem. 186, 123-128 (1989) or any references as described therein.

In one example, the present invention contemplates that the starter cells are incubated in the presence of a modulator of 5′AMP-activated protein kinase or AMPK for a time and under condition for time sufficient to render the cells capable of being differentiated into a plurality of different cell types. It will be apparent to the skilled artisan that the time of incubation in the presence of a modulator 5′AMP-activated protein kinase or AMPK may vary according to cell type and/or according to the type of modulator, and it is well within the ken of a skilled addressee to determine such parameters without undue experimentation. For example, the time of incubation in the presence of the modulator of 5′AMP-activated protein kinase or AMPK is between about 5 min and about 48 hours. Preferably, the time of incubation in the presence of the modulator is between about 5 min and about 24 hours, or preferably between about 5 min and about 15 hours, or preferably between about 5 min and 10 hours, or preferably between about 5 min and about 8 hours, or preferably between about 5 min and about 6 hours, or preferably between about 5 min and about 4 hours, or preferably between about 5 min and about 3 hours, or preferably between about 5 min and about 2 hours, or preferably between about 5 min and about 1 hour, or preferably between about 10 min and about 1 hour, or preferably between about 15 min and about 30 min. The person skilled in the art would appreciate that production of progenitor cells may continue albeit at below optimum even after the 48 hours incubation in the presence of a modulator of 5′AMP-activated protein kinase or AMPK, however such sub-optimum incubation conditions are clearly within the scope of the invention.

1.3. Incubation with a Phorbol Ester or Active Derivative Thereof.

Without being bound by any theory or mode of action, the inventor reasoned that incubating cells in the presence of a phorbol ester or active derivative thereof causes physiological changes in cells that induce the cells to trans-differentiate into progenitor cells capable of being differentiated into a plurality of different cell types by modulation of PKC function or a component thereof and/or the PKC signalling pathway. Alternatively or in addition thereto, without being bound by any theory or mode of action the inventor has also reasoned that incubating cells in the presence of a phorbol ester or active derivative thereof causes physiological changes in cells that induce the cells to trans-differentiate into progenitor cells capable of being differentiated into a plurality different cell types by cellular mechanisms independent of PKC e.g., by activating SRC or AP1 activator protein 1 (AP1) or any component(s) thereof.

In one example, the method for producing a progenitor cell that is capable of being differentiated into a plurality of different cell types comprising incubating differentiated cells in media comprising a phorbol ester active derivative thereof may comprise activating PKC thereby increases or decreasing the rate at which PKC phosphorylates any one of its numerous protein targets. The term is not limited by the mechanism underlying how the rate at which PKC phosphorylates any one of its numerous protein targets is increased or decreased. The potential mechanisms through which such a compound may act include, but are not limited to, allosteric mechanisms that affect, directly or indirectly, PKC activity, as well as mechanisms that act, directly or indirectly, to promote the phosphorylation of the PKC catalytic subunit catalyzed by upstream kinase.

The present invention contemplates any phorbol ester or active derivative thereof that is capable of trans-differentiating already differentiated cells into progenitor cells capable of differentiating into different cell types. Such phorbol ester or active derivative thereof, are either naturally occurring or synthetic and are known in the art or to be developed in the future. Preferred phorbol esters or active derivatives thereof suitable for this purposes are described herein and include but not limited to e.g., phorbol esters wherein the ester group is formate, acetate, propionate, butyrate, pentanoate, hexanote, benzoate or phenylacetate ester. In one example the phorbol ester or active derivative thereof includes, but is not limited to 4β-12-O-tetradecanoylphorbol-13-acetate (PMA); 4β-phorbol-12,13-dibutyrate (PDBu); 12-O-[2-methylaminobenzoate]-4-deoxy-13-acetate-14-deoxy phorbol, (phorbol sapintoxin A) or 12-O-[2-methylaminobenzoate]-4-hydroxy-13-acetate-14-deoxy phorbol (phorbol sapintoxin D); 13-O-Acetylphorbol-20-(9Z,12Z-octadecadienoate); 12-O-Decanoylphorbl-13-(2-methylbutyrate); 12-O-Acetylphorbol-13-decanoate; 12-O-(2-Methylbutyroyl)phorbol-13-dodecanoate; 12-O-Decanoylphorbol-13-acetate; 13-O-Acetylphorbol; Phorbol-12,13,20-triacetate; Phorbol-12,13,20-tribenzoate; Phorbol-12,13-diacetate-20-linoleate; 12-tetradecanoylphorbl-13-O-acetate; 12-O-Tetradecanoylphorbol-13,20-diacetate; 3-Deoxo-3,3-hydroxyphorbol-12,13,20-triacetate; 4-O-Methylphorbol-12,13,20-triacetate; Phorbol-4,9,12,13,20-pentaacetate; 13-O-Acetylcrotophorbolone-enol-20-linoleate; 4α-Phorbol-12,13,20-triacetate; 4α-Phorbol-12,13,20-tributyrate; 4α-Phorbol-4,12,13,20-tetraacetate; Lumiphorbol-12,13,20-triacetate; thymeleatoxin; resiniferatoxin, or any stereo-isomers thereof.

A method for incubating cells with PMA includes as described in Willem et al., Am J Physiol Cell Physiol 275:120-129 (1998) or any references as described therein or in Rebois, R. V., and Patel, J., J. Biol. Chem., 260:8026 (1985) or any references as described therein or in Farrar, J. J., et al., J. Immuno. 125:2555 (1980) or any references as described therein or in Schuman, L. D., et al., Cancer Lett., 47:11 (1989) or any references as described therein or in Pineiro V. et al., Biochemical and Biophysical Research Communications 252:345-347 (1998) or any references as described therein. A method for incubating cells with PDBu includes as described in Hyeon Ho Kim et al., Leukemia Research 29:1407-1413 (2005) or any references as described therein or in Sarker M. et al., Oncogene 21:4323-4327 (2002) or any references as described therein, or in Dykes, A. C, et al., Am J Physiol Cell Physiol 285:C76-C87 (2003) or any references as described therein. A method for incubating cells with phorbol sapintoxin A includes as described in Brooks et al., Cancer Lett. December; 38(1-2):165-7 (1987) or any references as described therein, or in Vartanian et al., FEBS letters 456:175-180 (1999) or any references as described therein or in Duyster et al., Bichem. J. 292:203-207 (1993) or any references as described therein.

In another example, incubation with a phorbol ester or active derivative thereof includes activating a receptor that modulates a cellular factor e.g., a protein kinase by contacting the receptor of a starter cell with a peptide, a polypeptide, a chemical, a nucleic acid, an antibody, an antibody fragment or a small molecule, such that the receptor activation modulates a cell signalling pathway initiates trans-differentiation of differentiated cells into progenitor cells capable of differentiating into other cell types. Preferably, the receptor is a receptor of a phorbol ester or active derivative thereof such as PKC, or a nonkinase phorbol ester receptor {alpha} 1-chimerin, or the presynaptic phorbol ester receptor Munc13-1, or the GABAA receptors (GABARs) or any phorbol ester known in the art or may become known in the future.

It will be understood that a phorbol ester or active derivative thereof falling with the scope of the invention exclude activators of the Akt/(PKB) pathway including but not limited to e.g., interleukin-1 (IL-1), platelet derived growth factor (PGDF-BB), insulin growth factor (IGF-1), transforming growth factor-beta (TGF-β), nerve growth factor (NGF) and carbachol or any active fragment or active chemical group thereof. It is also understood that a phorbol ester or active derivative thereof falling with the scope of the invention exclude activators of the NF-κB pathway including but not limited to e.g., tumor necrosis factor-alpha (TNF-α), interleukin 1 (IL-1), or any active fragment thereof, lysophosphatidic acid (LPA) or lipopolysaccharide (LPS).

In one example, the present invention contemplates that the starter cells are incubated in the presence of a phorbol ester or active derivative thereof for a time and under condition for time sufficient to render the cells capable of being differentiated into a plurality of different cell types. It will be apparent to the skilled artisan that such incubation period may vary according to the cell type and/or the phorbol ester or active derivative thereof used in the method of the invention, and it is well within the ken of a skilled addressee to determine such parameters without undue experimentation. For example, the time of incubation in the presence of a phorbol ester or active derivative thereof is between about 1 min and about 72 hours. Preferably, the time of incubation in the presence of the phorbol ester or active derivative thereof is between about 10 min and about 48 hours or between about 10 min and about 24 hours, or between about 10 min and about 15 hours, or between about 10 min and 10 hours, or between about 5 min and about 9 hours, or between about 10 min and about 8 hours, or between about 10 min and about 7 hours, or between about 10 min and about 6 hours, or between about 10 min and about 5 hours, or between about 10 min and about 4 hour, or between about 10 min and about 3 hours or about 10 min and about 2 hours, or about 10 min and about 60 min, or about 10 min and about 40 min, or about 10 min and about 30 min, or about 10 min and about 15 min. The person skilled in the art would appreciate that production of progenitor cells may continue albeit at below optimum even before the 10 min incubation in the presence of a phorbol ester or active derivative thereof or after the 72 hours incubation in the presence of a phorbol ester or active derivative thereof, however such sub-optimum incubation conditions are clearly within the scope of the invention.

1.4. Incubation with a Retinoid

Without being bound by any theory or mode of action, the inventor reasoned that incubating cells in the presence of a retinoid causes physiological changes in cells that induce the cells to trans-differentiate into progenitor cells capable of undergoing subsequent differentiation into a plurality of different cell types by modulation of one or more of the many pathways that may be regulated by retinoic acid receptors. The inventor reasoned that such retinoic acid receptors modulate expression of retinoic acid responsive or dependent target gene(s) by binding to retinoic acid response elements (RARE) located in the promoter regions or enhancers of target genes, thereby regulating transcription of the target genes.

In one example, the method for producing a progenitor cell that is capable of being differentiated into a plurality of different cell types comprising incubating differentiated cells in media comprising a retinoid may comprise modulating the function of a receptor or a ligand of retinoic acid thereby increases or decreasing the rate at which the receptor or ligand binds to any one of any one of its numerous gene or protein targets. The term is not limited by the mechanism underlying how the rate at which the receptor or ligand of retinoic acid any one of its numerous protein or gene targets is increased or decreased. The potential mechanisms through which such a compound may act include, but are not limited to, allosteric mechanisms that affect, directly or indirectly, the receptor or ligand activity, as well as mechanisms that act, directly or indirectly, to promote the phosphorylation of the receptor catalytic subunit that may be catalyzed by any upstream kinase.

The present invention contemplates any retinoid capable of binding to a retinoic acid receptor or ligand or that is capable of modulating e.g., by agonising or antagonising a receptor or ligand of retinoic acid or capable of mimicking modulation of a receptor or ligand of a retinoic acid and is capable of trans-differentiating already differentiated cells into progenitor cells capable of undergoing subsequent differentiation into a plurality of different cell types. Such retinoids are either naturally occurring or synthetic and are known in the art or to be developed in the future. Preferred retinoids suitable for this purposes are described herein and include but not limited to all-trans-retinoic acid (ATRA), 9-cis retinoic acid (9CRA), 13-cis retinoic acid, 11-cis retinoic acid, Am80, BMS189452, CD666, BMS188649, BMS185411, BMS188649, CD336/Am580, CD2019, CD437/AHPN, CD2665, CD2503, CD367, CD2314, CD 3640, AGN193109, or any stereo-isomers thereof.

In one example, retinoids that agonise a retinoic acid receptor or ligand include for example ATRA, 9CRA, 13-cis retinoic acid, 11-cis retinoic acid, Am80, BMS189452, CD666, BMS188649, BMS185411, BMS188649, CD336/Am580, CD2019, CD437/AHPN, CD367, CD2314, CD 3640, or any stereo-isomers thereof.

In one example, retinoids that antagonise a retinoic acid receptor or ligand include for example, AGN193109, CD2503 and CD2665, or any stereo-isomers thereof.

The present invention further includes retinoids from any of the aforementioned sources that may be purchased from any commercial source including Sigma Chemical Co., (St Louis, Mo.), Bristol-Myers Squibb (Buffalo, N.Y.) and Galderma Laboratories (Sophia, France).

A method for incubating cells with ATRA (e.g., Sigma) includes for example as described in Roy et al., Mol. Cell. Biol., 15(2):6481-6487 (1995) or any references as described therein, or in Boylan et al., Mol. Cell. Biol., 15(2):843-8517 (1995) or any references as described therein, or in Reshma et al., Proc. Natl. Acad. Sci. USA 92:7854-7858 (1995) or any references as described therein. A method for incubating cells with 9CRA (e.g., Sigma) includes for example as described in Roy et al., Mol. Cell. Biol., 15(2):6481-6487 (1995) or any references as described therein. A method for incubating cells with Am80 (e.g., Galderma Laboratories) includes for example as described in Roy et al., Mol. Cell. Biol., 15(2):6481-6487 (1995) or any references as described therein or in Takeda et al., Arterioscler. Thromb. Vasc. Biol. 26:1177-1183 or any references as described therein or in Fujiu et al., Circ. Res. 97:1132-1141 (2005). A method for incubating cells with BMS189452 (e.g., Bristol-Myers Squibb) includes for example as described in Roy et al., Mol. Cell. Biol., 15(2):6481-6487 (1995) or any references as described therein or in Marklund et al., Development 131:4323-4332 (2004) or any references as described therein. A method for incubating cells with CD666 (e.g., Galderma Laboratories) includes for example as described in Roy et al., Mol. Cell. Biol., 15(2):6481-6487 (1995) or any references as described therein or in Million et al., Am. J. Respir. Cell Mol. Biol., 25:744-750 (2001) or any references as described therein. A method for incubating cells with BMS188649 (e.g., Bristol-Myers Squibb) includes for example as described in Roy et al., Mol. Cell. Biol., 15(2):6481-6487 (1995) or any references as described therein. A method for incubating cells with BMS185411 (e.g., Bristol-Myers Squibb) includes for example as described in Yong Zhuang et al., Mol. Canc. Res., 1:619-630 (2003) or any references as described therein. A method for incubating cells with CD336/Am580 (e.g., Galderma Laboratories) includes for example as described in Marchetti et al., Canc. Res., 59:6257-6266 (1999) or any references as described therein or in Million et al., Am. J. Respir. Cell Mol. Biol., 25:744-750 (2001) or any references as described therein. A method for incubating cells with CD2019 (e.g., Galderma Laboratories) includes for example as described in Million et al., Am. J. Respir. Cell Mol. Biol., 25:744-750 (2001) or any references as described therein. A method for incubating cells with CD437/AHPN (e.g., Galderma Laboratories) includes for example as described in Marchetti et al., Canc. Res., 59:6257-6266 (1999) or any references as described therein. A method for incubating cells with CD2665 (e.g., Galderma Laboratories) includes for example as described in Marchetti et al., Canc. Res., 59:6257-6266 (1999) or any references as described therein. A method for incubating cells with CD2503 (e.g., Galderma Laboratories) includes for example as described in Million et al., Am. J. Respir. Cell Mol. Biol., 25:744-750 (2001) or any references as described therein. A method for incubating cells with CD367 (e.g., Galderma Laboratories) includes for example as described in Christina Zechel., Mol. Endocrinol., 19(6):1629-1645 (2005) or any references as described therein. A method for incubating cells with CD2314 (e.g., Galderma Laboratories) includes for example as described in Christina Zechel., Mol. Endocrinol., 19(6):1629-1645 (2005) or any references as described therein or in Yong Zhuang et al., Mol. Canc. Res., 1:619-630 (2003) or any references as described therein. A method for incubating cells with CD3640 (e.g., Galderma Laboratories) includes for example as described in Christina Zechel., Mol. Endocrinol., 19(6):1629-1645 (2005) or any references as described therein. A method for incubating cells with AGN193109 (e.g., Galderma Laboratories) includes for example as described in Christina Zechel., Mol. Endocrinol., 19(6):1629-1645 (2005) or any references as described therein or in Soprano et al., Toxicol. Appl. Pharmacol. 174:153-159 (2001).

In another example, incubation with a retinoid includes activating a receptor or a ligand of retinoic acid that modulates a cellular factor e.g., a protein kinase by contacting the receptor or ligand of a starter cell with a peptide, a polypeptide, a chemical, a nucleic acid, an antibody, an antibody fragment or a small molecule, such that the receptor activation modulates a cell signalling pathway initiates trans-differentiation of differentiated cells into progenitor cells capable of differentiating into other cell types. Preferably, the receptor or ligand is a receptor or ligand of retinoic acid include but not limited to nuclear receptors for retinoic acid e.g., retinoic acid receptors (RARs) such as RARα, RARβ, RARγ, or the retinoic X receptors (RXRs) such as RXRα, RXRβ, RXRγ, or cellular retinoic acid binding proteins (CRABPs) such as CRABP-I or CRABP-II, or glycoprotein 130 (gp13), or soluble Intercellular adhesion molecule-I (ICAM-I) or Vascular cell adhesion molecule-1 (VCAM-1) or any other retinoic acid receptor or ligand known in the art or may become known in the future.

It will be understood that a retinoid falling with the scope of the invention exclude activators of the Akt/(PKB) pathway including but not limited to e.g., interleukin-1 (IL-1), platelet derived growth factor (PGDF-BB), insulin growth factor (IGF-1), transforming growth factor-beta (TGF-β), nerve growth factor (NGF) and carbachol or any active fragment or active chemical group thereof. It is also understood that a retinoid falling with the scope of the invention exclude activators of the NF-κB pathway including but not limited to e.g., tumor necrosis factor-alpha (TNF-α), interleukin 1 (IL-1), or any active fragment thereof, lysophosphatidic acid (LPA) or lipopolysaccharide (LPS).

In one example, the present invention contemplates that the starter cells are incubated in the presence of a retinoid for a time and under condition for time sufficient to render the cells capable of being differentiated into a plurality of different cell types. It will be apparent to the skilled artisan that such incubation period may vary according to the cell type and/or the retinoid used in the method of the invention, and it is well within the ken of a skilled addressee to determine such parameters without undue experimentation. For example, the time of incubation in the presence of a retinoid is between about 24 hours i.e., 1 day and about 240 hours i.e., 10 days. Preferably, the time of incubation in the presence of the retinoid is between about 48 hours i.e., 2 days and about 240 hours i.e., 10 days, or 48 hours i.e., 2 days and about 216 hours i.e., 9 days, or 48 hours i.e., 2 days and about 192 hours i.e., 9 days, or 48 hours i.e., 2 days and about 168 hours i.e., 7 days, or 48 hours i.e., 2 days and about 144 hours i.e., 6 days, or between about 48 hours i.e., 2 days and about 120 hours i.e., 5 days, or between about 48 hours i.e., 2 days and about 96 hours i.e., 4 days, or between about 48 hours and 72 hours i.e., 3 days, or between about 248 hours i.e., 1 day and about 48 hours i.e., 2 days. The person skilled in the art would appreciate that production of progenitor cells may continue albeit at below optimum even before the 24 hours incubation in the presence of a retinoid or after the 240 hours incubation in the presence of a retinoid, however such sub-optimum incubation conditions are clearly within the scope of the invention.

### 1.5. Induction of the Akt/(PKB) Pathway

In one example, the method to induce the Akt/(PKB) pathway in a starter cell may comprise contacting the starter cell with any one or more factors that induce(s) the Akt/(PKB) signaling pathway. For example, an inducer initiates and/or enhances Akt/(PKB) pathway signaling in a starter cell. Such an inducer is also referred to as an Akt/(PKB) pathway enhancer or an Akt/(PKB) pathway agonist. For example, the inducer is a peptide, a polypeptide, a chemical, a nucleic acid, an antibody, an antibody fragment or a small molecule, or any insult that induces cellular stress such as, but not limited to, hypoxia, or UV irradiation.

The present invention contemplates any inducer of the Akt/(PKB) signaling known in the art or to be developed in the future. Preferably, the inducer of Akt/(PKB) signaling includes, but is not limited to factors such as: platelet derived growth factor (PGDF-BB), insulin growth factor (IGF-1), transforming growth factor-beta (TGF-β), nerve growth factor (NGF) and carbachol, pyruvate, cytokines such as IL-1, or any active fragment or active chemical group thereof. A method for inducing the Akt/(PKB) pathway with PDGF-BB includes as described in Li et al., Mol. Biol. Cell 15:294-309 (2004) or Gao et al, J. Biol. Chem. 280:9375-9389 (2005) or any references as described therein. A method for inducing the Akt/(PKB) pathway by co-activation with carbachol and NGF includes as described in Wu and Wong Cellular Signalling 18:285-293 (2006) or any references as described therein. A method for inducing the Akt/(PKB) pathway with IGF-1 includes as described in Kulik and Weber Mol. Cell. Biol. 18:6711-6718 (1998) or any reference as described therein. A method for the induction of the Akt/(PKB) pathway by TGF-β includes as described in by Conery et al., Nat Cell Biol (2004) δ: 366-72 or as described by Horowitz et al., J. Biol. Chem. 279: 1359-1367 (2004) or any reference as described therein. Other methods for inducing the Akt/(PKB) pathway with these factors includes methods as described in any one of the Examples or as described in Song et al., J. Cell. Mol. Med. 9:59-7 (2005); Dillon et al., Oncogene 26:1338-1345 (2007) or any reference as described therein.

In another example, the inducer of Akt/(PKB) signaling includes activating a receptor that initiates and/or enhances the Akt/(PKB) signaling pathway by contacting the receptor of a starter cell with a peptide, a polypeptide, a chemical, a nucleic acid, an antibody, an antibody fragment or a small molecule, such that the receptor activation initiates and/or enhances the Akt/(PKB) signaling pathway.

In one such example, the receptor is a growth factor receptor such as IGF receptor tyrosine kinase, or the TGF-β type I serine/threonine kinase receptor, or the TGF-β type II serine/threonine kinase receptor, or TGF-β type III receptor, or any one of the integrin receptors, such as α2β1 α1β1, and αv 3, or a GPCR receptor, or a cytokine receptor such as the IL-1 receptor, or a B-cell receptor.

In another example, the inducer of Akt/(PKB) signaling includes activating any intracellular signalling intermediate that initiates and/or enhances the Akt/(PKB) signaling pathway by contacting a starter cell with a peptide, a polypeptide, a chemical, a nucleic acid, an antibody, an antibody fragment or a small molecule, such that the activation initiates and/or enhances the Akt/(PKB) signaling pathway. These intracellular intermediates include but are not limited to downstream signalling intermediates activated by growth factor receptors such as, GAB1, GAB2, IRS1, PI3K, PIP2, PIP3, ras, or downstream signalling intermediates activated by integrin receptors such as, FAK, paxillin, ILK, PI3K, PIP2, PIP3, or downstream signalling intermediates activated by cytokine receptors, such as, JAK1, PI3K, PIP3, PDK-1 or downstream signalling intermediates activated by B-cell receptors such as, BCAP, PI3K, PDK-1, downstream signalling intermediates activated by GPCR receptors such as, Gf3Gy/PI3K, PIP3, PDK-1.

A method to measure the activation of the Akt/(PKB) pathway includes any method that measures the activity of Akt/(PKB), or any known intracellular signaling intermediate of the Akt/(PKB), as described in Kulik and Weber Mol. Cell. Biol. 18:6711-6718 (1998), or any reference described therein. For example, the phosphorylation of Akt/(PKB) may be used as a marker of the activation of the pathway. The method to measure Akt/(PKB) phosphorylation is described in Kulik and Weber. Briefly, after incubation with factors to induce the Akt/(PKB) pathway, cells are placed on ice and lysed in 1% Nonidet P-40, 0.5% deoxycholate, 150 mM NaCl, and 20 mM HEPES supplemented with phosphatase and protease inhibitors. Insoluble material is pelleted by centrifugation at 10,000×g for 20 min, and the supernatants are equalized for protein concentration by the addition of NLB. Samples are subjected to Western Blot analysis by standard methods using a phospho-Akt (S473) specific antibody. The membrane is stripped and reprobed with Akt-specific antibodies.

### 1.6. Induction of the NF-κB Pathway

In one example, the method to induce the NF-κB pathway in a starter cell may comprise contacting the starter cell with any one or more factors that induce(s) the NF-κB signaling pathway in said primary cell, cell strain or cell line. For example, an inducer initiates and/or enhances NF-κB pathway signaling in a starter cell. Such an inducer is also referred to as an NF-κB pathway enhancer or an NF-κB pathway agonist.

The present invention contemplates any inducer of NF-κB signaling known in the art or to be developed in the future. For example, the inducer is a peptide, a polypeptide, a chemical, a nucleic acid, an antibody, an antibody fragment or a small molecule, or any insult that induces cellular stress such as, but not limited to, hypoxia, UV irradiation, or high cell density culturing, maintenance or incubation.

In one example, the inducer of NF-κB signaling includes, but is not limited to factors such as: tumor necrosis factor-alpha (TNF-α), interleukin 1 (IL-1), or any active fragment thereof, lysophosphatidic acid (LPA), pyruvate, or lipopolysaccharide (LPS). A method for inducing the NF-κB signaling pathway with TNF-α includes as described in Kouba et al., J. Biol. Chem. 276:6214-6244 (2001) or any reference as described therein. A method for inducing the NF-κB signaling pathway with IL-1 includes as described in Kessler et al., J. Exp. Med. 176:787-792 (1992) or any reference as described therein. A method for inducing the NF-κB signaling pathway with LPA includes as described in Shahrestanifar et al., J. Biol. Chem. 274:3828-3833 (1999) or any reference as described therein. Other methods for inducing the NF-κB signaling pathway with any of these factors includes as described in any one of the Examples.

In another example, the inducer of NF-κB signaling includes activating a receptor that initiates and/or enhances the NF-κB signaling pathway by contacting the receptor of a starter cell with a peptide, a polypeptide, a chemical, a nucleic acid, an antibody, an antibody fragment or a small molecule, or any insult that induces cellular stress such as UV irradiation or incubating, maintaining or incubating cells at high cell density, such that the receptor activation initiates and/or enhances the NF-κB signaling pathway.

In one preferred example, the inducer of NF-κB signaling comprises culturing, maintaining or incubating differentiated cells at high cell density conditions.

In one preferred example, the receptor is a cytokine receptor such as the IL-1 receptor, or the TNF receptor, or a growth factor receptor such as, the IGF receptor, or the LPS receptor, such as TLRs, or the T-cell receptor, or the B-cell receptor.

In another preferred example, the inducer of NF-κB signaling includes activating any intracellular signalling intermediate that initiates and/or enhances the NF-κB signaling pathway by contacting a starter cell with a peptide, a polypeptide, a chemical, a nucleic acid, an antibody, an antibody fragment or a small molecule, such that the activation initiates and/or enhances the NF-κB signaling pathway. These intracellular intermediates include but are not limited to downstream signalling intermediates activated by growth factor receptors, such as, PI3K, Akt/PKB, or by the TNF receptor(s) such as TRADD/RIP/FADD/TRAF, NIK/MEKK, or the cytokine receptors, such as TRAF6/MyoD/IRAK, IRAK/TRAF6, TAK1 or T-cell receptors such as, Vav/PKC/ZAP70, BIMP/BCL10/MALT or B-cell receptors such as, BLK/Lyn/Fyn, PKC, BIMP/BCL10/MALT.

A method to measure the activation of the NF-κB pathway includes any method that measures the activity of NF-κB, such as the translocation of NF-κB. Such methods are well known in the art and includes methods as described in Ding et al., J Biol Chem, 273:28897-28905 (1998) or any reference as described therein. Briefly, cells that have been induced in their NF-κB pathway are fixed with 4% formaldehyde in phosphate-buffered saline for 20 min at room temperature, permeabilized with 0.1% Triton X-100 in phosphate-buffered saline for 5 min at room temperature, and then washed twice with 0.1 M Tris-HCl buffer, pH 7.8. To block nonspecific antigenic sites, cells are incubated for 20 min with 5% non-fat dry milk in 0.1 M phosphate buffer, pH 7.8, at room temperature. Cells are washed two times in 0.1M Tris wash buffer, incubated for 1 h with rabbit anti-p65 NF-κB antibody (Santa Cruz Biotechnology, Inc., Santa Cruz, Calif.) diluted 1:2000 in 0.1 M phosphate buffer, pH 7.8, with 0.1% bovine serum albumin (fraction V; Sigma). The plates are washed three times in Tris wash buffer and incubated 30 min, room temperature, with a 10 μg/ml solution in water of biotinylated anti-rabbit IgG (Vector Laboratories, Burlingame, Calif.). The plates are washed three times in Tris wash buffer and incubated 30 min, room temperature, with 2.5 μg/ml solution of Texas Red avidin (Vector) in the phosphate/bovine serum albumin buffer. The cells are washed three times in Tris wash buffer and stored in 0.1 m Tris. Two hours prior to analysis a 1 μg/ml solution of Hoechst 33342 (Molecular Probes, Inc., Eugene, Oreg.) in phosphate-buffered saline is added to each well at room temperature, and the wells are scanned and analysed in the ArrayScan™ cytometer (Cellomics, Inc., Pittsburgh, Pa.).

### 2. Detachment of Adherent Cells in Culture

In accordance with the generality of the invention, the means by which adherent cells in culture are detached from each other and/or from the culture vessel may be varied.

In a one example, adherent cultures are detached from tissue culture plates by incubation of the adherent cells in trypsin for a time and under conditions sufficient for detachment to occur e.g., as described in the Examples.

Trypsin may be purchased from a variety of commercial sources in stock concentrations up to about 2.5% (w/v) trypsin, such as, for example, from GIBCO (Invitrogen). The final trypsin concentration used to achieve detachment when using such a solution is preferably about 0.01% (w/v) to about 0.25% (w/v) trypsin, including about 0.05% (w/v), or about 0.10% (w/v), or about 0.11% (w/v), or about 0.12% (w/v), or about 0.13% (w/v), or about 0.14% (w/v), or about 0.15% (w/v), or about 0.16% (w/v), or about 0.17% (v/v) or about 0.18% (w/v) or about 0.19% (w/v) or about 0.2% (w/v) or about 0.25% (w/v).

It will be apparent to the skilled artisan that the time of incubation in trypsin solution may vary according to cell type, and it is well within the ken of a skilled addressee to determine such parameters without undue experimentation. For example, the time of incubation in trypsin solution is sufficient for the cells to lift from the plates and/or preferably, to detach from each other as determined by the degree of cell clumping or aggregation.

It will also be apparent to the skilled artisan that the temperature for the incubation in trypsin solution is preferably between about 15° C. and about 37° C., or preferably room temperature, or more preferably 37° C. By “room temperature” is meant ambient temperature e.g., between about 18° C. and about 25° C.

Other suitable methods for achieving detachment of cells from each other and/or from the culture vessel include, but are not limited to, cold shock; treatments to release integrin receptors from the extracellular matrix, which comprises fibronectin, vitronectin, and one or more collagens; activation of degradation of matrix molecules including, but not limited to fibronectin, collagens, proteoglycans, and thrombospondin; inducing or enhancing the secretion of proteases such as, but not limited to collagenase, stromelysin, matrix-metalloproteinases (MMPs; a class of structurally related zinc-dependent endopeptidases that collectively degrade extracellular matrix components) or plasminogen activator; and decreasing or repressing the expression of protease inhibitors, plasminogen activator inhibitor (PAI-1) or tissue inhibitors of metalloproteinases (TIMPs). Such methods are described without limitation for example, by Ivaska and Heino, Cell. Mol. Life. Sci. 57:16-24 (2000), Nagase et al., Cardovasc. Res. 69(3):562-73 (2006) or a reference cited therein.

One example of cold shock means comprise incubating the cells in ice-cold phosphate buffered saline (PBS) or other isotonic buffer for a time and under conditions sufficient for detachment to occur. In one such preferred example, conditions include cold shock for about 10 minutes or until the cells lift from the plates and/or detach from each other as determined by the degree of cell aggregation.

A further means for achieving detachment of cells from each other and/or from the culture vessel includes incubating the cells in a citric saline (e.g., 0.135M potassium chloride, 0.015M sodium citrate). One example of citric saline treatment comprises incubating the cells and citric saline in PBS at 37° C. and decanting cells for a time and under conditions sufficient for cells to lift from the plates and/or detach from each other as determined by the degree of cell aggregation.

Integrin receptors can be released from the extracellular matrix by incubating the cells with a synthetic peptide containing the Arg-Gly-Asp sequence that competes for binding to the integrin receptors such as described, for example, by Haymen et al., Journal Cell Biol, 100:1948-1954 (1985). Alternatively, or in addition, integrin receptors are released from extracellular matrix by incubating cells in a Ca²⁺-free and Mg⁺-free solution comprising EDTA (e.g., Ca²⁺-free and Mg⁺-free PBS comprising EDTA, or other Ca²⁺-free and Mg⁺-free isotonic buffer) essentially as described by Sambrook, Fritsch & Maniatis, Molecular Cloning: A Laboratory Manual, Cold Spring Harbor Laboratories, New York, Second Edition (1989), whole of Vols I, II, and III; Animal Cell Culture: Practical Approach, Third Edition (John R.W. Masters, ed., 2000), ISBN 0199637970, whole of text; Methods In Enzymology (S. Colowick and N. Kaplan, eds., Academic Press, Inc.), whole of series.

In one preferred example, means for inducing or enhancing MMP expression include induction by addition of growth factor or cytokine to the culture medium.

The present invention clearly encompasses the use of any means by which adherent cells in culture are detached from each other and/or from the culture vessel as described by Ivaska and Heino, Cell. Mol. Life. Sci. 57:16-24 (2000) and references described therein.

### 3. Ligands of Protease Activated Receptors (PARs)

In an alternative example, adherent cultures are detached from tissue culture plates by incubation of the adherent cells in the presence of one or more PAR ligands for a time and under conditions sufficient for detachment to occur e.g., as described in the Examples.

By “protease-activated receptor” or “PAR” is meant any one of a class of G-protein coupled receptors including, but not limited to, the receptors designated PAR1, PAR2, PAR3, and PAR4, and combinations thereof.

Activation of a PAR by its cognate endogenous or non-endogenous ligand leads to a cascade of cellular events such as, for example, contraction of myometrium and/or vascular and/or smooth muscle and/or activation of mitogen-activated protein kinases as described e.g., by Shintani et al., British Journal of Pharmacology (2001) 133, 1276-1285 or Belham et al., Biochem J. 320: 939-946, 1996. Alternatively, or in addition, activation of PAR by the ligand may protect cells from apoptosis and/or activate the Akrt pathway and/or activate the NF-kappaB pathway. Activation of the Akrt pathway and/or activate the NF-kappaB pathway can be determined e.g., by detecting expression of one or more pathway intermediates in cells.

In one example, PAR ligands include, but are not limited to trypsin, tryptase, chymotrypsin, elastase, thrombin, plasmin, coagulation factor Xa, granzyme A and cathepsin G.

PAR ligands that are proteases can be purchased from a variety of commercial sources and used, for example at concentrations in the range of about 0.01% (w/v) to about 0.25% (w/v). It will be apparent to the skilled artisan that the time of incubation in a PAR ligand may vary according to cell type, and it well within the ken of a skilled addressee to determine such parameters without undue experimentation. For example, the time of incubation is sufficient for activation of one or more downstream cellular effects of the receptor to occur, as determined by routine procedures. It will also be apparent to the skilled artisan that the temperature for the incubation in PAR ligand is preferably between about 15° C. and about 37° C., or preferably room temperature, or more preferably 37° C.

For example, Ishii et al., J. Biol. Chem. 270 (27):16435-16440 (1995) describe a method for activating PAR using thrombin. Thrombin may be purchased from a variety of commercial sources, e.g., Sigma, and the final thrombin concentration is preferably in the range of about 10 nM to about 100 nM thrombin, including 10 nM thrombin, or 20 nM thrombin, or 30 nM thrombin, or 40 nM thrombin, or 50 nM thrombin, or 60 nM thrombin, or 70 nM thrombin, or 80 nM thrombin, or 90 nM thrombin, or 100 nM thrombin. Preferably, thrombin is diluted in phosphate-buffered saline optionally comprising about 0.5% (v/v) polyethylene glycol 8000. Preferably, cells are incubated with thrombin at about 25° C. for about 60 min.

In another example, Quinton et al., J. Biol. Chem. 279 (18); 18434-18439 (2004) describe activation of PAR using plasmin.

In other examples, PAR can be activated by any one of the methods described by Shintani et al., British Journal of Pharmacology (2001) 133, 1276-1285 or Wang et al., Biochem. J. 408: 221-230 (2007), or Dery et al., Am. J. Physiol. 274 (Cell Physiol. 43): C1429-1452, incorporated herein by reference.

In another example, to activate any one of the PAR receptors the adherent cells are incubated in the presence of a known GPCR receptor agonist.

### 4. Storage of Cells

In a preferred example, the progenitor cells prepared according to the invention are stored in a suitable media conditions until required for differentiation. Preferably, where the differentiated cells are optionally exposed to prolonged incubation in low serum media, the progenitor cells prepared according to the invention are stored in low-serum medium until required for differentiation. Alternatively, the cells are stored in medium containing serum, e.g., DMEM-HG containing 10% FCS. In one example, where the cells are further cultured, maintained or incubated under high density conditions the progenitor cells prepared according to the invention are optionally stored in a high density plating medium capable of supporting progenitor cells until further required.

Optionally, the cells are stored in low serum conditions at 4° C. for a short time. For example, the cells may be stored on ice for 1 min to 6 hours.

Optionally, the cells are cryogenically frozen in liquid nitrogen. The method used to freeze the cells in optimal freezing media and conditions will be apparent to the skilled artisan and is dependent on the cell type. For example, such methods are commercially available from cell suppliers such as American Type Culture Collection (Rockville, Md.) or PromoCell® (Banksia Scientific Company, QLD). Methods that are used are also described in Animal Cell Culture: Practical Approach, Third Edition (John R. W. Masters, ed., 2000), ISBN 0199637970.

### 5. Differentiation

The present invention contemplates that the cells prepared according to the invention are differentiated into any other differentiated cell type. For example, a cell type of a tissue that is required for regeneration. The tissue may be a tissue of any part of the body including but not limited to organs such as cardiovascular tissue, skin, bone, gut, stomach, pancreas, thymus, thyroid, eye, spleen, heart, blood vessels, cardiovascular, blood, bone marrow, or any nervous tissue.

Preferably, the cells of the invention are differentiated to, but not limited to: skin cells, epidermal cells, keratinocytes, and melanocytes, and epithelial cells, cardiovascular tissue cells such as cardiomyocytes, cardiac muscle cells, cardiac fibroblasts, and neural cells such as those derived from the peripheral nervous system (PNS) and central nervous system (CNS) including, but not limited to, glial cells, such as, e.g., Schwann cells, astrocytes, oligodendrocytes, microglial cells, and blood cells, such as lymphocytes, including T cells and B cells, macrophages, monocytes, dendritic cells, Lagerhans cells, eosinophils, and the like, adipocytes, osteoclasts, osteoblasts, endocrine cells, β-islet cells of the pancreas, endothelial cells, epithelial cells, granulocytes, hair cells, mast cells, myoblasts, Sertoli cells, striated muscle cells, zymogenic cells, oxynitic cells, brush-border cells, goblet cells, hepatocytes, Kupffer cells, stratified squamous cells, pneumocytes, parietal cells, podocytes, synovial cells, such as synovial fibroblasts, serosal cells, pericytes, chondrocytes, osteocytes, Purkinje fiber cells, myoepithelial cells, megakaryocytes, and the like.

Methods for differentiating cells of the invention include, but are not limited to the methods described in any one of the Examples or an example herein. The present invention also contemplates the differentiation of further cell types including, but not limited to the following.

### Neural Tissue Development

To differentiate cells prepared according to the invention, cells that have been de-differentiated according to any example as described herein are suspended in Neuroblast A medium (Invitrogen/GIBCO) supplemented with 5% horse serum, 1% fetal calf serum, L-glutamine (2 mM), transferrin (100 μg/ml), insulin (2 μg/ml), retinoic acid 0.5 mM, brain-derived neurothrophic factor (10 ng/ml), and then allowed to attach, i.e. are plated onto tissue culture plates in said medium for a time sufficient to differentiate the cells to a neural phenotype.

### Dopamine-Secreting Issue Development

To differentiate cells prepared according to the invention, cells that have been de-differentiated according to any example as described herein are first suspended in dopaminergic induction media (DMEM serum free medium supplemented with 2 mM glutamine, 100 μg/ml streptomycin, 100 U/ml penicillin, 12.5 U/ml nystatin, N2 supplement (Invitrogen, New Haven, Conn.), and 20 ng/ml fibroblast growth factor-2 (FGF-2) and epidermal growth factor (EGF) (both from R&D Systems, Minneapolis, Minn.) for 2-3 days. The medium is then changed to basal induction medium containing Neurobasal and B27 (both from Invitrogen), in addition to 1 mM dibutyryl cyclic AMP (db cAMP), 3-isobutyl-1-methylxanthine (IBMX), and 200 μM ascorbic acid (all from Sigma, St Louis, Mo.) and brain-derived neurotrophic factor (BNDF) 50 ng/ml (Cytolab, Rehovot, Israel), as described in Barzilay et al., Stem cells and Development 17:547-554, 2008 which is herein incorporated by reference. The cells are then allowed to attach, i.e. are plated onto tissue culture plates in said medium for a time sufficient to differentiate the cells to a dopamine secreting phenotype,

### Skeletal/Cardiac Muscle Development

To differentiate cells prepared according to the invention, cells that have been de-differentiated according to any example as described herein are suspended in alpha-Modification of Eagle's Medium supplemented with 10% fetal calf serum, L-glutamine (2 mM), ascorbate-2-phosphate (100 μM/ml), and 5-azacytodine (5 μM/ml) and then allowed to attach, i.e. are plated onto tissue culture plates in said medium for a time sufficient to differentiate the cells to a skeletal/muscle phenotype.

### Epithelial Development

To differentiate cells prepared according to the invention, cells that have been de-differentiated according to any example as described herein are suspended in keratinocyte basal medium (Clonetics) supplemented with Bovine Pituitary Extract (50 μg/ml), epidermal growth factor (10 ng/ml), Hydrocortisone (0.5 μg/ml), Insulin (5 μg/ml) and then allowed to attach, i.e. are plated onto tissue culture plates in said medium for a time sufficient to differentiate the cells to a keratinocyte lineage.

### Osteoblasts, Tendon, Ligament or Odontoblast Development

To differentiate cells prepared according to the invention, cells that have been de-differentiated according to any example as described herein are suspended in alpha-Modification of Eagle's Medium supplemented with 10% fetal calf serum, L-glutamine 2 mM, ascorbate-2-phosphate (100 μM), Dexamethasone (10⁻⁷M) and BMP-2 (50 ng/ml) and then allowed to attach, i.e. are plated onto tissue culture plates in said medium for a time sufficient to differentiate the cells.

### Pericyte or Smooth Muscle Cell Development

To differentiate cells prepared according to the invention, cells are suspended in alpha-Modification of Eagle's Medium supplemented with 10% fetal calf serum, L-glutamine 2 mM, ascorbate-2-phosphate (100 μM), platelet derived growth factor-BB (10 ng/ml) then layered over 200 μl of matrigel in 48-well plates for a time sufficient to differentiate the cells.

### Assessment of the Differentiated Phenotype

A method to assess the lineage of differentiated cells of the invention includes, but is not limited to use of commercially available antibodies and flow cytometry. This procedure has been reported previously and is well known in the art. Briefly, differentiated cell cultures are liberated by trypsin/EDTA digest then incubated for 30 min on ice. Approximately 2×10⁵ cells are washed then resuspended in 200 μl of primary antibody cocktail for 1 hr on ice. The primary antibody cocktail comprises of saturating concentrations of a mouse IgG monoclonal antibody or rabbit IgG for each tube (Table 1). Antibodies for the markers listed in Table 1 are commercially available from a variety of sources including but not limited to DAKO, Santa Cruz, Pharmingen, or Sigma. For the staining with antibodies reactive with intracellular antigens the cells are first washed with PBS then permeablized by treatment with 70% ethanol on ice for ten minutes then washed prior to staining. The mouse isotype IgM and IgG negative control Mabs are treated under the same conditions. Following incubation with primary antibodies, cells are washed and exposed to saturating levels of goat anti-mouse IgM μ-chain specific-FITC ( 1/50 dilution) and either goat anti-mouse IgG γ-specific-PE ( 1/50 dilution) or anti-rabbit Ig-specific-PE ( 1/50 dilution) (Southern Biotechnology Associates) in a final volume of 100 μl. The cells are incubated for 45 min on ice, then washed twice then fixed in FAX FIX (PBS supplemented with 1% (v/v), 2% (w/v) D-glucose, 0.01% sodium azide). Flow cytometric analysis is performed using a FACSCalibur flow cytometer and the CellQuest software program (Becton Dickinson Immunocytometry Systems, San Jose, Calif.). Data analysis is performed using CellQuest and the Modfit LT V2.0 software program (Verity Software House, Topsham, Me.).

TABLE 1
Markers for lineage identification

| | |
|---|---|
| 1. | Skeletal Muscle: Myo E Desmin |
| 2. | Smooth Muscle: SMMHC, SMHC-FAST, alphaSMAC, PDGF-R, |
| | Vimentin; |
| 3. | Chondrocytes: Type II Collagen; Collagen IX; Aggrecan; Link |
| | Protein; S100; Biglycan; |
| 4. | Basal Fibroblasts: Laminin; Type IV Collagen; Versican; |
| 5. | Endothelial Cells: vWF; VCAM-1; Endoglin; MUC18; CD31; |
| | CD34; SDF-1 |
| 6. | Cardiomyocytes: Calponin; Troponin I; Troponin C; |
| 7. | Neurons: NCAM; GFAP; Neuroanalase; Neurofilament; |
| 8. | Bone: AP, Type I Collagen; CBFA 1; OCN; OPG; RANKL; |
| | Annexin II |
| 9. | Fat: CEPBalpha; PPARgamma; Leptin; |
| 10. | Epithelial cells: Keratin 14; Cytokeratin 10 + 13; EGFR; |
| 11. | Fibroblast: Collagen III; NGFR; Fibroblast marker; |
| 12. | Haematopoietic: CD14; CD45; Glycophorin-A. |

### 6. Formulations and Treatments

Pharmaceutical compositions and other formulations for application to the human or animal body e.g., for stimulating or enhancing tissue repair in a subject, are suitable for use topically, systemically, or locally as an injectable and/or transplant and/or device, usually by adding necessary buffers.

Preferred formulations for administration, the non-culture expanded cells used in this invention are in a pyrogen-free, physiologically acceptable form.

The cells may be injected in a viscous form for delivery to the site of tissue damage.

Topical administration may be suitable for wound healing and tissue repair.

In one example, therapeutically useful agents may also optionally be included in the progenitor cell formulation, or alternatively, administered simultaneously or sequentially with the composition in the methods of the invention.

In another example, the compositions of the present invention may be used in conjunction with presently available treatments for tendon/ligament injuries, such as suture (e.g., vicryl sutures or surgical gut sutures, Ethicon Inc., Somerville, N.J., USA) or tendon/ligament allograft or autograft, in order to enhance or accelerate the healing potential of the suture or graft.

The choice of a carrier material is based on biocompatibility, biodegradability, mechanical properties, cosmetic appearance and interface properties. The particular application of the progenitor cells will generally define the appropriate carrier. In one example, cells are mixed with a matrix, preferably a biodegradable matrix or a matrix comprised of pure proteins or extracellular matrix components. Other useful matrices include e.g., collagen-based materials including sponges, such as Helistat™ (Integra LifeSciences, Plainsboro, N.J., USA), or collagen in an injectable form, and sequestering agents such as hyalouronic acid-derived materials. Biodegradable materials, such as cellulose films, or surgical meshes, may also serve as matrices. Such matrices may be sutured into an injury site, or wrapped around a site of injury such as a tendon or ligament. Another preferred class of carriers includes polymeric matrices, wherein the progenitor cell of the invention is mixed with a polymer of poly lactic acid, poly glycolic acid, or a copolymer of lactic acid and glycolic acid. These matrices may be in the form of a sponge, or in the form of porous particles, and may also include a sequestering agent. Suitable polymer matrices are described, for example, in WO93/00050.

In another example, the formulations of progenitor cells of the invention may comprise other therapeutically useful agents such as, for example, one or more cytokines, chemokines, leukemia inhibitory factor (LIF/HILDA/DIA), migration inhibition factor, MP52, growth factors including epidermal growth factor (EGF), fibroblast growth factor (FGF), platelet derived growth factor (PDGF), transforming growth factors (TGF-alpha and TGF-beta), and fibroblast growth factor-4 (FGF-4), parathyroid hormone (PTH), insulin-like growth factors (IGF-I and IGF-II), or combinations thereof.

In another example, the formulation comprises at least one other agent that promotes hematopoiesis, such as, for example a cytokine, which participates in hematopiesis. Some non-limiting examples are: CSF-1, G-CSF, GM-CSF, interleukins, interferons, or combinations thereof.

In another example, the formulation comprises at least one other agent that promotes the delivery of systemic proteins such as Factor IX, VIII, growth hormone etc.

In another example, the progenitor cells are genetically engineered to express a protein of interest prior to the application to the subject in need. The protein of interest is any macromolecule, which is necessary for cell growth, morphogenesis, differentiation, tissue building or combinations thereof. These are, for example, a bone morphogenic protein, a bone morphogenic-like protein, an epidermal growth factor, a fibroblast growth factor, a platelet derived growth factor, an insulin like growth factor, a transforming growth factor, a vascular endothelial growth factor, cytokines related to hematopoiesis, factors for systemic delivery as such as GH, factor VIII, factor IX or combinations thereof.

The term “cells engineered to express a protein of interest” is defined hereinabove as a cell or to a tissue which had been modified via molecular biologic techniques, for example via recombinant DNA technology, to express any macromolecule which is necessary for cell growth, morphogenesis, differentiation, tissue building or combinations thereof. In another example, cells are thus modified in order to produce an increased amount of any macromolecule, which is necessary for cell growth, morphogenesis, differentiation, tissue building or combinations thereof.

The step of genetically engineering a cell to express a protein of interest is preferably performed by the transfection or transduction of the cell with a nucleic acid encoding the protein of interest.

The term “transfection” or “transfected cells” refer to cells in which DNA is integrated into the genome by a method of transfection, i.e. by the use of plasmids or liposomes.

The term “transduction” or “transduced cells” refers to viral DNA transfer for example, by phage or retroviruses. The nucleic acid, which encodes the protein of interest, can be introduced by a vector molecule, as well, and represents an additional example of this invention.

The vector molecule can be any molecule capable of being delivered and maintained, within the target cell, or tissue such that the gene encoding the product of interest can be stably expressed. In one example, the vector utilized in the present invention is a viral or retroviral vector or a non-viral DNA plasmid. According to one aspect, the method includes introducing the gene encoding the product into the cell of the mammalian tissue for a therapeutic or prophylactic use. The viral vectors, used in the methods of the present invention, can be selected from the group comprising of (a) a retroviral vector, such as MFG or pLJ; (b) an adeno-associated virus; (c) an adenovirus; and (d) a herpes virus, including but not limited to herpes simplex 1 or herpes simples 2 or (e) lentivirus. Alternatively, a non-viral vector, such as a DNA plasmid vector, can be used. Any DNA plasmid vector known to one of ordinary skill in the art capable of stable maintenance, within the targeted cell, or tissue upon delivery, regardless of the method of delivery utilized is within the scope of the present invention. Non-viral means for introducing the gene encoding for the product into the target cell are also within the scope of the present invention. Such non-viral means can be selected from the group compriseing of (a) at least one liposome, (b) Ca₃(PO₄)₂, (c) electroporation, (d) DEAE-dextran, and (e) injection of naked DNA.

The term “nucleic acid” refers to polynucleotides or to oligonucleotides such as deoxyribonucleic acid (DNA), and, where appropriate, ribonucleic acid (RNA) or mimetics thereof. The term should also be understood to include, as equivalents, analogs of either RNA or DNA made from nucleotide analogs, and, as applicable to the example being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, enhanced cellular uptake, enhanced affinity for nucleic acid target and increased stability in the presence of nucleases.

The formulations of the invention are useful for treating cartilaginous tissue, defects of the embryonic joint where tendon, ligaments, and bone form simultaneously at contiguous anatomical locations, regenerating tissue at the site of tendon attachment to bone, or for wound healing, such as skin healing and related tissue repair. Types of wounds include, but are not limited to burns, incisions and ulcers.

The formulations of the invention are also useful for tissue renewal or regeneration that ameliorates an adverse condition of tissue, degeneration, depletion or damage such as might be caused by aging, genetic or infectious disease, accident or any other cause, in humans, livestock, domestic animals or any other animal species.

In another example the formulations of the invention are also useful for promoting tissue development in livestock, domestic animals or any other animal species in order to achieve increased growth for commercial or any other purpose.

In another example the formulations of the invention are also useful in plastic surgeries, such as, for example, facial or body reconstruction.

In another example the formulations of the invention are also useful for enhancing repair of tissue injuries, tears, deformities or defects, and for the prophylaxis or prevention of tissue damage.

In another example, the formulations of the invention are also useful for treating and/or preventing osteoporosis, which results from a decrease in estrogen, which may be caused by menopause or ovariectomy in women. Use of the progenitor cells of the present invention for prevention of accelerated bone resorption and inhibition of a decrease of bone volume, bone quality and bone strength is also provided by the invention. Trabecular connectivity and trabecular unconnectivity may be maintained at healthy levels with the pharmaceutical compositions of the present invention. Osteoporosis and its symptoms such as decreased bone volume, bone quality, and bone strength, decreased trabecular connectivity, and increased trabecular unconnectivity may be treated or prevented by administration of a pharmaceutically effective amount of the pharmaceutical composition to a patient in need thereof.

In another example, the formulations of the invention are also useful for regenerating tissues which have been damaged through acute injury, abnormal genetic expression or acquired disease. In one such example, the formulations of the invention are also useful for regenerating cardiac tissue such as a cardiac muscle.

In another example, the formulations of the invention are also useful for stimulating skeletal development in livestock, domestic animals or any other animal species in order to achieve increased growth for commercial or any other purpose.

In another example, the formulations of the invention are also useful for treatment of neoplasia or hyperplasia of bone or cartilage or any other tissue, in humans, livestock, domestic animals or any other animal species.

In another example, the formulations of the invention are also useful for stimulating haematopoiesis e.g., in combination with hematopoietic transplants.

The dosage regimen, which is the amount of the cells that are administered in order to obtain a therapeutic effect, is affected by various factors which modify the action of the progenitor cells' composition, e.g., amount of tissue desired to be repaired or formed, the site of injury or damage, the condition of the damaged tissue, the size of a wound, type of damaged tissue, the patient's age, sex, and diet, the severity of any infection, time of administration and other clinical factors. The dosage may vary with the type of matrix used in the reconstitution and the types of additional proteins in the composition. The addition of other known growth factors, such as IGF-I (insulin like growth factor I), to the final composition, may also affect the dosage. Progress can be monitored by periodic assessment of tissue formation and/or growth and/or repair. The progress can be monitored by methods known in the art, for example, X-rays (CT), ultra-sound, MRI, arthroscopy and histomorphometric determinations.

### 7. Tissue and Organ Building, Repair and Regeneration

The present invention encompasses the use of the progenitor cells prepared according to the invention or differentiated cells derived there from for building, repairing or regenerating a tissue, and/or building, repairing or regenerating an organ. It is apparent that when progenitor cells are used in this example, those progenitor cells are differentiate in situ during the tissue/organ building, repair or regeneration, whereas differentiated cells derived from the progenitor cells are not required to differentiate in situ.

It is also apparent that when differentiated cells are employed in this example, multiple cell types may be required to build, regenerate or repair tissues comprising different cell types in nature, or whole organs. One or more, or all, of these different cell types may be produced in accordance with the present invention by employing appropriate differentiation media and conditions. A plurality of progenitor cell populations may each be derived from different starting cells or cell types, or produced in different batches. Similarly, a plurality of differentiated cells may comprise different batches of the same cell type and/or different cell types per se produced from the same or different batches of progenitor cells or the same or different starting cell types.

The organ that is produced, repaired or regenerated is without limitation and includes e.g., skin, bone, gut, stomach, pancreas, thymus, thyroid, eye, spleen, heart, blood vessels, cardiovascular, bone marrow, or nervous system, a cardiovascular organ such as artery. The tissue may be any tissue without limitation including e.g., a tissue of any one or more of the foregoing organs and further includes skin, muscle, fat, bone, or any tissue derived from the group of endoderm, mesoderm, ectoderm or combination thereof and including cartilage, connective tissue, tendon, nerve adipose, gastrointestinal tissue, cardiac tissue such as of the heart, cornea, optical tissue, exocrine and/or endocrine glands. For example if subcutaneous fatty tissue were to be regenerated it would include the regeneration of the primary cell type i.e. fat, and its blood supply (vascular tissue) nerve supply and stromal tissue (supporting structures including ECM, basil lamina etc). Similarly this concept can be used to support the regeneration of most tissues e.g. for muscle it will be myocytes, vascular supply and nerve supply and stromal tissue.

In one example, the tissue and/or organ to be regenerated may be tissue and/or organ injured, lost, or atrophied by disease processes or degeneration. Such tissues and/or organs could be the spinal cord (for example, multiple sclerosis), the substantia nigra in Parkinson's disease, or the olfactory mucosa or Alzheimer's disease, a cardiac muscle or cardiovascular organ such as heart such as after myocardial infraction. It will be understood that progenitor cells of the present invention may be provided to individuals predisposed to any condition resulting in tissue and/or organ loss, injured or atrophied such as multiple sclerosis, Parkinson's or Alzheimer's disease, cancer, cardiac injury such as myocardial infraction or to individuals having symptoms of onset of these diseases for preventing or reducing the severity of these diseases.

In one example, the progenitor cells prepared according to any example hereof are used to build, repair or regenerate a tissue and/or organ or an element of a tissue and/or organ e.g., in situ at a site of injury to a tissue and/or organ. In one example, such regeneration is achieved by providing the progenitor cells at least one of a neuropeptide Y (NPY), a fragment or variant of neuropeptide Y, a compound capable of inducing expression of a gene encoding a neuropeptide Y protein or fragment or variant thereof, a cell that produces a neuropeptide Y and/or an agonist or antagonist of a neuropeptide Y receptor to induce building, repair or regeneration of a tissue and/or organ e.g., at the site of injury, as described in International Application PCT/AU2006/000481 filed Apr. 10, 2006 (Publication No. WO/2006/108218) which is incorporated herein by reference in its entirety. In one preferred example, the fragment or variant of neuropeptide Y is biologically functional. The progenitor cells may be provided or administered directly to a site of injury in the tissue and/or organ. Alternatively, or in addition, the progenitor cells are produced in situ as described according to any example hereof.

Alternatively, or in addition, progenitor cells prepared in accordance with any example hereof are used to build, repair or regenerate a tissue or organ or an element of a tissue and/or organ e.g., in situ at a site of injury in a tissue and/or organ. In one example, such regeneration is achieved by providing the progenitor cells at least one of a neuregulin, a fragment of a neuregulin, a compound capable of inducing expression of a neuregulin gene, and/or an agonist or antagonist of a receptor for neuregulin to induce building, repair or regeneration of a tissue or organ e.g., at the site of injury, substantially as described in International Application PCT/AU2007/000238 filed Feb. 28, 2007 (Publication No. WO/2007/098541) which is incorporated herein by reference in it entirety. In one preferred example, the fragment of neuregulin is biologically functional. The progenitor cells may be provided or administered directly to a site of injury in the tissue and/or organ. Alternatively, or in addition, the progenitor cells are produced in situ as described according to any example hereof.

Alternatively, or in addition, the progenitor cells prepared by the methods according to any example hereof are used to build, repair or regenerate a tissue and/or organ or an element of a tissue and/or organ e.g., in situ at a site of injury in the tissue or organ. In one example, such regeneration is achieved by providing the progenitor cells at least one of a neurotrophin, a fragment of a neurotrophin, a compound capable of inducing expression of a neurotrophin gene, and/or an agonist or antagonist of a receptor for a neurotrophin to induce building, repair or regeneration of a tissue or organ e.g., at the site of injury, substantially as described in International Application PCT/AU2007/000238 filed Feb. 28, 2007 (Publication No. WO/2007/098541) which is incorporated herein by reference in it entirety. Non-limiting examples of neurotrophin(s) suitable for use in the present invention include nerve growth factor (NGF), neurotrophic factor 3 (NT-3), brain derived neurotrophic factor (BDNF), neurotrophic factor 4 (NT-4), neurotrophic factor 5 (NT-5) or Ciliary Neurotrophic Factor CNTF. In a particularly preferred example, the neurotrophin is NGF. In one example, the fragment of neurotrophin is biologically functional. The progenitor cells may be provided or administered directly to a site of injury in the tissue and/or organ. Alternatively, or in addition, the progenitor cells are produced in situ as described according to any example hereof.

In another example, one or more populations (or batches) of progenitor cells or one or more populations of differentiated cells derived from the progenitor cells as described according to any example hereof is cultured or perfused onto a scaffold or matrix that allows the cells to develop into a tissue or organ or part thereof e.g., a biocompatible scaffold or matrix such as a biodegradable scaffold matrix.

In another example, building or regenerating an organ or multi-layered tissue such as an artificial organ or tissue may be achieved by a process comprising:
(i) perfusing a first population of progenitor cells produced in according with any example hereof or differentiated cells derived therefrom into and/or onto a first side of a biocompatible scaffold or matrix such that the cells attach to the matrix and then culturing the cells for a time and under conditions sufficient to produce a first specialized tissue layer; and
(ii) perfusing a second population of undifferentiated or differentiated cells distinct from the cells at (i) into and/or onto a second side of the biocompatible matrix such that the second population of cells attaches to the matrix and then culturing the second population of cells in the matrix for a time and under conditions sufficient to produce a second specialized tissue layer that is different from the first specialized tissue layer
   to thereby create a multi-layered tissue and/or organ construct.

This process may be achieved by reversing the order of (i) and (ii).

In another example, building or regenerating an organ or multi-layered tissue such as an artificial organ or tissue may be achieved by a process comprising:
(i) perfusing a first population of progenitor cells produced in according with any example hereof or differentiated cells derived therefrom into and/or onto a first side of a biocompatible scaffold or matrix such that the cells attach to the matrix and then culturing the cells for a time and under conditions sufficient to produce a first specialized tissue layer; and
(ii) perfusing a second population of progenitor cells produced in according with any example hereof or differentiated cells derived therefrom into and/or onto a second side of the biocompatible matrix such that the second population of cells attaches to the matrix and then culturing the second population of cells in the matrix for a time and under conditions sufficient to produce a second specialized tissue layer that is different from the first specialized tissue layer
   to thereby create a multi-layered tissue and/or organ construct.

In another example, a multi-layered tissue and/or organ construct can also be created by culturing first and second populations of cells on the same side of the biocompatible matrix.

In another example, different populations of cells are cultured simultaneously or sequentially in and/or on the matrix.

In accordance with these examples, perfused cells are cultured until they differentiate and/or proliferate to produce a first monolayer comprising cells with a desired phenotype and morphology. Once the first monolayer has attained a desired cell density, a second layer of the same cell population is deposited on the first monolayer. The second layer of perfused cells is cultured under conditions to provide nutrients to both the second cell layer and the first monolayer and for time sufficient for cells in the layers to form a bilayer having cells with a desired cell type and morphology. The process is repeated until a poly-layer comprising a plurality of cell monolayers of the desired cell type and morphology is produced. Polylayers may also be produced by layering of multiple bilayers, trilayers, etc.

In another example, the invention provides a tissue construct or organ construct comprising a biocompatible scaffold or matrix perfused with at least one population of progenitor cells of the present invention and/or one or more populations of differentiated cells derived from progenitor cells of the invention. The tissue or organ construct may comprise one or a plurality of cell types or populations or batches e.g., a plurality of cell types on the same or different sides of the biocompatible scaffold or matrix.

As used herein, the term “scaffold” or “matrix” shall be taken to mean any material in and/or on which cells may differentiate and/or proliferate to form a tissue or organ or part thereof. Accordingly, a scaffold or matrix provides the structure or outline to the tissue or organ to be repaired, regenerated or built. A scaffold or matrix will generally be a three-dimensional structure comprising a non-degradable or a biodegradable material, e.g., a decellularized organ or part thereof, that can be shaped into a desired tissue or organ. For example, a scaffold or matrix also provides sufficient interstitial distances required for cell-cell interaction.

As used herein, the term “biocompatible scaffold” or “biocompatible matrix” shall be taken to mean a scaffold or matrix as hereinbefore defined that, with any tissue and/or organ proliferating or growing thereon, is further suitable for implantation into a host subject. When grown in a biocompatible matrix, the proliferating cells mature and segregate properly to form tissues analogous to counterparts found in vivo. In other examples, counter parts tissues or organs present in vivo may be replaced by a tissue and/or organ repaired, regenerated or repaired by the method described herein.

A biocompatible scaffold or matrix is generally a polymeric composition e.g., polyglycolic acid, or the infra-structure of an organ following decellularization i.e., removal of substantially all cellular material. Non-limiting examples of biocompatible polymeric matrixes can be formed from materials selected from the group comprising of, but are not limited to, cellulose ether, cellulose, cellulosic ester, fluorinated polyethylene, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyglycolic acid, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, and copolymers or physical blends thereof. The polymeric matrix can be coated with a biocompatible and biodegradable shaped setting material. In one example, the shape settling material is a liquid copolymer e.g., poly-DL-lactide-co-glycolide. In another example, the scaffold or matrix comprises synthetic or semi-synthetic polymer fibers e.g., Dacron™, Teflon or Gore-Tex™.

Preferred non-toxic biocompatible scaffolds or matrices may be made of natural or synthetic polymers, such as, for example, collagen, poly(alpha esters) such as poly(lactate acid), poly(glycolic acid) (PGA), polyorthoesters and polyanhydrides and their copolymers, which degraded by hydrolysis at a controlled rate and are reabsorbed. These materials provide the maximum control of degradability, manageability, size and configuration. Preferred biodegradable polymer material include polyglycolic acid and polygalactin, developed as absorbable synthetic suture material. Polyglycolic acid and polygalactin fibers may be used as supplied by the manufacturer. Other biodegradable materials include cellulose ether, cellulose, cellulosic ester, fluorinated polyethylene, phenolic polymer, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends of these materials.

Decellularized scaffolds or matrices are produced by a process in which the entire cellular and tissue content is removed, leaving behind a complex infra-structure e.g., comprising a fibrous network of stroma or unspecialized connective tissue that predominantly comprises collagen and/or proteoglycan. Decellularized structures can be rigid or semi-rigid. Methods of producing decellularized matrix or scaffold are described e.g., in U.S. Pat. No. 7,354,702 and U.S. Pat. No. 7,429,490, both of which are incorporated herein by reference.

Scaffolds or matrices may be impregnated with suitable antimicrobial agents and may be colored by a color additive to improve visibility and to aid in surgical procedures.

In one preferred example, the biocompatible polymer is a synthetic absorbable polygalactin material or polyglycolic acid (PGA) fibers (Ethicon Co., Somerville, N.J.; Craig P. H., et al. Surg. 141; 1010 (1975) or Christenson L, et al., Tissue Eng. 3 (1): 71-73; discussion 73-76 (1997)) which can be used as supplied by the manufacturer. This biocompatible polymer may be shaped using methods such as, for example, solvent casting, compression molding, suturing, filament drawing, meshing, leaching, weaving and coating (See Mikos, U.S. Pat. No. 5,514,378, hereby incorporated by reference).

In some examples, the polymers are coated with compounds such as basement membrane components, agar, agarose; gelatin, gum arabic, collagens, such as collagen types I, II, III, IV, and V, fibronectin, laminin, glycosaminoglycans, mixtures thereof, and other hydrophilic and peptide attachment materials having properties similar to biological matrix molecules known to those skilled in the art of cell culture.

Factors, including nutrients, growth factors, inducers of differentiation or dedifferentiation, products of secretion, immunomodulators, inhibitors of inflammation, regression factors, biologically active compounds which enhance or allow ingrowth of the lymphatic network or nerve fibers, and drugs, can be incorporated into the matrix or provided in conjunction with the matrix. Similarly, polymers comprising peptides such as the attachment peptide RGD (Arg-Gly-Asp) can be synthesized for use in forming matrices. Angiogenesis factors, cytokines, extracellular matrix components, and other bioactive materials or drugs may also be impregnated into the scaffold or matrix at any stage preceding implantation e.g., to promote repair, grafting, or reduce or inhibit rejection. Growth factors include e.g., epidermal growth factor (EGF), vascular endothelial growth factor (VEGF), heparin-binding epidermal-like growth factor (HBGF), fibroblast growth factor (FGF), cytokines, genes, proteins, and the like. Other useful additives include antibacterial and antifungal agents to promote healing by suppression of infections. For example, the biocompatible matrix can be fabricated to have a controlled pore structure that allows such nutrients to permeate or contact the perfused cells in the absence of significant cell migration through the pores. In vitro cell attachment and cell viability can be assessed using scanning electron microscopy, histology and quantitative assessment with radioisotopes.

In another example, additional collagenous layers may be added to the inner surfaces of the decellularized structure to create a smooth surface as described in International PCT Publication No. WO 95/22301, the contents of which are incorporated herein by reference. This smooth collagenous layer promotes cell attachment which facilitates growth and development. As described in International PCT Publication No WO 95/22301, this smooth collagenous layer may be made from acid-extracted fibrillar or non-fibrillar collagen, which is predominantly type I collagen, but may also include type II collagen, type IV collagen, or both. The collagen used may be derived from any number of mammalian sources, typically pig and cow skin and tendons. The collagen for example has been processed by acid extraction to result in a fibril dispersion or gel of high purity. Collagen may be acid-extracted from the collagen source using a weak acid, such as acetic, citric, or formic acid. Once extracted into solution, the collagen can be salt-precipitated using NaCl and recovered, using standard techniques such as centrifugation or filtration. Details of acid extracted collagen are described, for example, in U.S. Pat. No. 5,106,949 issued to Kemp et al., incorporated herein by reference.

The present invention will now be illustrated by the following Examples, which are not intended to be limiting in any way. The teachings of all references cited herein are incorporated herein by reference.

### Example 1

### Preparation of Cells Having the Ability to Differentiate into Cells of Osteogenic and Adipogenic Lineages by Treatment with a Protease: Method 1

In a first set of experiments for producing cells having the ability to differentiate into different cell types, fibroblasts are cultured and then either incubated in media comprising trypsin. The cells produced by this method are then tested for their ability to differentiate into cells of osteogenic lineage as determined by expression of alkaline phosphatase (ALP) being a well established marker of bone differentiation and cells produced by this method are also tested for their ability to differentiate into adipocytes, as determined by the accumulation of fat. Differentiation into osteogenic cells and adipocytes is selected in these primary experiments because methods for such differentiation are well-established.

### Cells

Human dermal fibroblasts (HDF's) are purchased from Promocell. Cells are cultured and expanded in DMEM-HG (Biowhittaker)+10% fetal calf serum (FCS) (Invitrogen). Human mesenchymal stem cells (MSC's) (Promocell) are used as positive controls. Cells are cultured in 5% CO2, and at 37° C. until confluence. Both HDF's and MSC's cells are detached at confluence using 0.12% trypsin, 0.02% EDTA and 0.04% glucose, and detached cells are assessed for differentiation into osteogenic cells and adipocytes.

Production of Cells Capable of Differentiating into Other Cell Types and Osteogenic Culture

HDF's and MSC's are seeded at 25,000 cells per well (n=6) in a 96 well plate. Osteogenic media (OM) consisted of low glucose containing Medium 199 and 10% FCS containing 20 μg/ml ascorbic acid phosphate-magnesium salt, 1.5 mg/ml beta glycerophosphate and 40 ng/ml dexamethasone. Control media (CM) consisted of DMEM-HG+10% FCS. All cells are detached on day 16 using 0.12% trypsin, after 15 days of treatment to assess alkaline phosphatase expression (ALP). All reagents are purchased from Sigma-Aldrich, Australia unless otherwise indicated.

Differentiation into Osteogenic Cells

Cell suspensions of detached fibroblasts are centrifuged at 800 g for 6 minutes, and supernatant discarded. The detached cells are then re-suspended in 200 μl per well of either CM or OM according to three culture groups. The 3 groups are: 1) Detached cells are seeded and maintained in CM (HDF CM); 2) Detached HDF's cells are exposed to CM and then maintained in OM (HDF CM ΔOM) according to standard protocol for osteogenic differentiation; 3) Detached cells are exposed to OM and maintained in OM (HDF OM ΔOM), which is an improved culture protocol. MSC's that are detached and seeded in OM are used as positive controls. Experiments are repeated in triplicate.

### Assessment of Osteogenesis Using an Alkaline Phosphatase (ALP) Assay

After incubation in CM or OM as described above for 15 days, ALP expression is assessed. The culture media is removed, and cells are washed in PBS. Following this, 50 μl of 10% (v/v) Passive Lysis Buffer (PLB) (Promega) in dH₂0 is added per well. 96 well plates are placed in a water bath sonicator for 10 minutes (Elma). After sonication, the lysate is separated into two equal samples of 25 μl each. An aliquot of 75 μl of p-Nitrophenyl phosphate (pNPP) is added to each well of the other 96 well plate added to the remaining sample and is incubated for 120 minutes at 37° C. 100 μl of 2M NaOH is added following incubation. The absorbance of pNP (yellow) is measured by optical plate reader at 405 nm against a standard curve of pNP using Versamax plate reader and Softmax Pro software (Molecular Probes).

Production of Cells Capable of Differentiating into Other Cell Types and Adipogenic Culture

Adipogenic media (AM) consists of Medium 199 containing 170 nM insulin, 0.5 mM 3-isobutyl-1-methylxanthine, 0.2 mM indomethacin, 1 μM dexamethasone, and rabbit serum (15% v/v). HDF's are cultured and expanded to confluence as described above. All cells are detached using 0.12% trypsin. Cell suspensions are centrifuged at 800 g for 6 minutes, and supernatant discarded. Cells are then resuspended in 200 μl per well of either CM or AM. Detached cells are seeded at 50,000 cells per well (n=6) in a 96 well plate, to assess adipocyte morphology. Experiments are repeated in triplicate.

Differentiation into Adipocytes

To assess the effects of cellular detachment on adipogenesis, HDF's cells are exposed to 3 culture groups: 1) Detached cells seeded in CM (HDF CM); 2) Detached cells seeded in CM, and changed to AM on day 2 (HDF CM Δ AM); 3) Detached cells exposed to AM and maintained in OM (HDF AM Δ AM) which is the improved protocol. MSC's are used as positive controls for cell morphology. All cells are maintained under culture conditions for 15 days, and stained on day 16 with oil Red O.

### Assessment of Adipogenesis

Cells counts are assessed for adipocyte formation using light microscopy and visual detection of oil red staining. Adipocyte formation usually occurred in positive controls within 5-7 days of exposure to adipogenic medium. Oil red staining is performed by fixing cells in paraformaldehyde (4%/PBS) for 1 hour, then washing cells with isopropanol (60% v/v) and stained with a working solution of Oil Red 0 solution for 10 minutes. The working solution of Oil red 0 is prepared by dissolving 4.2 g of Oil red 0 in 1200 ml absolute isopropanol and leaving the solution overnight and filtering through analytical filter and then adding 900 ml of dH₂0.

### Example 2

### Preparation of Cells Having the Ability to Differentiate into Adipocytes by Treatment with a Protease: Method 2

In this set of experiments for producing cells having the ability to differentiate into different cell types, fibroblasts are cultured and then either incubated in media without trypsin or comprising trypsin. The cells produced by this method are then tested for their ability to differentiate into adipocytes, as determined by the accumulation of fat.

### 2.1 Materials and Methods

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 10,000 to 20,000 cells per well or about 370.87 to 740.74 cells per mm² surface area. Test cells are plated onto larger plates at the same concentration of cells per well or cells per mm² surface area. Once all cells are attached, test cells are detached by the addition of 20 μl of detachment solution comprising 0.12% trypsin, 0.02% EDTA and 0.04% glucose (SAFC Biosciences, Cat #59430C) and incubated at 37° C. until cells lifted from the plates. Test cells are recovered from culture, then diluted to 200 to 400 μl with DMEM-HG (e.g., Lonza, Cat #12-604) (10% FBS) and maintained in this medium until required for re-differentiation but without re-attachment or adherence of the cells to each other or to the culture vessel. Control cells are not detached, and are used directly in the differentiation assay as described below.

Differentiation into Adipocytes

Cells at a density of about 20,000 cells per well or about 740.74 cells per mm² surface area of the well are incubated in adipogenic medium (Medium 199 comprising 170 nM insulin, 0.5 mM 3-isobutyl-1-methylxanthine, 0.2 mM indomethacin, 1 μM dexamethasone, and 15% rabbit serum) for about 12-21 days. Adipogenic media is replaced every 3 days on both test and control cells.

### Assessment of Adipogenesis

After incubation for about 12-21 days in adipogenic medium, the medium is removed, and cells are fixed in 10% formaldehyde solution in aqueous phosphate buffer for at least 1 hour. Cells are then washed with 60% isopropanol and stained with a working solution of Oil Red 0 solution (in 60% isopropanol, see below for preparation) for 10 minutes. The cells are then washed several times with water, and destained in 100% isopropanol for 15 minutes. The destain solution is removed and the optical density of the solution is measured at 500-510 nm.

The working solution of Oil red O is prepared as previously described (Humason 1972) by dissolving 4.2 g of Oil red O in 1200 ml absolute isopropanol and left overnight without stirring at room temperature. The solution is filtered through analytical filter paper 589-WH (Schleicher and Schuell); after filtration, 900 ml of distilled water is added and the solution left overnight at 4° C. without stirring and subsequently filtered twice. This working solution can be stored at room temperature and has a shelf life of 6-8 months.

For example, production of progenitor cells by the method described herein, may be enhanced by agonism of the Akt/(PKB) and/or the NF-κB pathway by incubation of the differentiated cells in the presence of an agonist compound as described in any one of the examples 10 to 21, and/or by incubation under high serum conditions as described in example 3.

### Example 3

### Preparation of Cells Having the Ability to Differentiate into Adipocytes by Treatment with Protease and Seeding Cells at High Density in a High Density Plating Medium

In a this set of experiments for producing cells having the ability to differentiate into different cell types, fibroblasts are either incubated in media without trypsin or containing trypsin and then cultured at different lengths of time under high density conditions of about 100,000 cells per well/plate or at about 3703.7 cells per mm² surface area of the well/plate before adherence of the cells in a high density plating medium. The cells produced by this method are then tested for their ability to differentiate into adipocytes, as determined by the accumulation of fat.

### 3.1 Materials and Methods

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Cells are cultured onto 96-well plates or onto 12 well Flexiperm® chamber on glass slide (27 mm² plating surface area), or 8 well Nunc Latek II chamber slide (54 mm² plating surface). Control cells are plated directly onto 96 well plates at about 20,000 cells per well or about 740.74 cells per mm² surface area of the well/plate. Test cells are plated onto larger plates but at the same concentration of cells per mm² surface area. Once all cells are attached and reach sub-confluence or confluence, the cells are detached as described below. Alternatively the cells are washed with PBS and then the medium is replaced with DMEM-HG (e.g., Lonza Cat #12-604) or M199 supplemented with 10% FBS for different periods of incubation time, from 10 to 14 days.

At the conclusion of the incubation period, test cells are detached by the addition of 20 to 40 μl of detachment solution containing 0.12% trypsin, 0.02% EDTA and 0.04% glucose (SAFC Biosciences, Cat #59430C) and incubated at 37° C. until cells lifted from the plates. Control cells that are not treated with trypsin are used directly in the differentiation assay as described below.

Test cells are recovered from culture and seeded at concentrations of about 100,000 cells per well/plate or at about 3703.7 cells per mm² surface area of the well/plate before attachment of the cells to the plate/well directly in 200 to 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum) for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation. Cells are then transferred to an adipogenic medium (Medium 199 containing 170 nM insulin, 0.5 mM 3-isobutyl-1-methylxanthine, 0.2 mM indomethacin, 1 μM dexamethasone, and 15% rabbit serum) and allowed to expand for about 10-21 days.

As a negative control for the production of progenitor cells, trypsinized cells are seeded at a reduced density i.e., about 740.1 cells per mm² surface area, in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and incubated as for samples seeded at high density e.g., for up to about 24 hours or until adherence is achieved and/or as determined by analysis of cell marker expression and/or by the ability of aliquots of cells to subsequently undergo differentiation. The high density plating medium is then replaced with 200 to 400 μl adipogenic media and cells are allowed to expand for about 10-21 days.

As a negative control for differentiation, trypsinized cells are seeded at high density in high density plating medium (e.g., DMEM-HG supplemented with 10% FBS) and incubated as for test samples seeded at high density e.g., for up to about 24 hours or until adherence is achieved and/or as determined by analysis of cell marker expression and/or by the ability of aliquots of cells to subsequently undergo differentiation. The high density plating medium is then replaced with 200 to 400 μl DMEM-HG medium and cells are allowed to expand for about 10-21 days.

As positive control for differentiation, rat bone marrow stromal/stem cells (rBMSCs) are expanded in DMEM medium containing L-Glutamine and 10% FCS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 400 μl DMEM-HG containing 10% FCS for up to about 24 hours or until adherence is achieved. The medium is replaced with 200 to 400 μl adipogenic medium (Medium 199 containing 170 nM insulin, 0.5 mM 3-isobutyl-1-methylxanthine, 0.2 mM indomethacin, 1 μM dexamethasone, and 15% rabbit serum) and cells are allowed to expand for about 10-21 days. Medium is replaced every 3 days for both test cells and negative and positive control cells. Assessment of adipogenesis is carried out as described in Example 2.

### 3.2 Results

Differentiation potential of control cells compared to test cells at each day post-incubation under high density conditions of about 100,000 cells per well/plate or at about 3703.7 cells per mm² surface area of the well/plate before adherence of the cells in a an adipogenic medium is measured by an assessment of adipogenesis as described above.

Cells that are not incubated in the presence of trypsin do not produce detectable adipocytes. In contrast, fibroblasts that are incubated in the presence of trypsin for a time and under conditions sufficient to detach the cells from each other and from the culture plate and then directly seeded in high density plating medium under high density conditions before adherence, are capable of differentiating into adipocytes when cultured in adipogenic medium for varying lengths of time. Adipocytes are apparent when seeding test cells in high density directly into high density plating medium occurred within 6 hours of incubation of the cells with trypsin. Although adipocytes are apparent in cultures incubated in adipogenic medium from a period of only about 1 day in, the optimum time for assuming this ability to differentiate e.g., into adipocytes, is about 10-14 days, as determined by assaying the numbers of fat-producing cells at each time point in the 12-day period assayed (from day 10 to day 21 in adipogenic medium). As will be apparent from the disclosure herein, lower numbers of progenitor cells may be apparent with shorter or longer periods of culture, maintenance and incubation of the cells in differentiation media than are observed herein, however such sub-optimum incubation conditions are clearly within the scope of the invention.

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 4

### Preparation of Cells Having the Ability to Differentiate into Adipocytes by Incubation in Low Serum Medium Combined with Treatment with Protease and Incubation at High Cell Density Conditions

To improve the yield of cells having the ability to differentiate into other cell types, the inventor sought to investigate the effect of incubation of differentiated cells in low serum media on plasticity. Specifically, the inventor sought to test whether or not the additional step of incubating cells in a low serum media may produce equivalent or improved results as the combined action of incubation in the presence of protease and culture, maintenance and incubation of cells at high cell density in high density plating media or alternatively by agonism of the Akt/(PKB) and/or the NF-κB pathway using an agonist compound. Without being bound by any theory or mode of action, the inventor reasoned that low-serum incubation conditions for 5-9 days further induces/enhances activation of the Akt/(PKB) and/or the NF-κB pathway. An advantage of using low-serum incubation for 5-9 days in concert with detachment of cells and high density incubation conditions in high density plating medium and/or inducing the Akt/(PKB) and/or the NF-κB pathway using an agonist compound, is an increase in proportion of cells achieving optimum plasticity produced by the method of the invention.

Production of Cells Capable of Differentiating into Other Cell Types

Fresh human dermal fibroblasts derived from adult skin or from foreskin described in Example 1, are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and are incubated at 37° C. in a humidified atmosphere of 5% CO₂ in air until adherent. The cells are then washed with PBS and then the medium is replaced with M199 supplemented with 0-1% FBS for different periods of incubation time, from 5 to 14 days. Control cells as washed with PBS and then the medium is replaced with M199 supplemented with 10% FBS for different periods of incubation time, from 5 to 14 days.

At the conclusion of the incubation period in low-serum media, test and control cells are detached by the addition of 20 to 40 μl of detachment solution containing 0.12% trypsin, as in example 1 and incubated at 37° C. until cells lifted from the plates. Test cells are recovered from culture within about 4-6 hours after trypsinization and seeded at concentrations of about 100,000 cells per well/plate or at about 3703.7 cells per mm² surface area of the well/plate before attachment of the cells to the plate/well directly in 200 to 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum), for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation. Cells are then transferred to an adipogenic medium (Medium 199 containing 170 nM insulin, 0.5 mM 3-isobutyl-1-methylxanthine, 0.2 mM indomethacin, 1 μM dexamethasone, and 15% rabbit serum) and allowed to expand for about 10-21 days, as described in example 2. Negative and positive controls are also set out as described in example 2.

Medium is replaced every 3 days for both test cells and negative and positive control cells. Adipogenesis is assessed by staining cells with Oil Red O solution as described in Example 1 above.

Although adipocytes are apparent in cultures incubated from a period of only about 5-7 days in the differentiation media, the optimum time for assuming this ability to differentiate e.g., into adipocytes, is about 5-9 days, as determined by assaying the numbers of fat-producing cells at each time point in the 11-day period assayed (from day 10 to day 21). The person skilled in the art would appreciate that differentiation of test cells into adipocytes may continue albeit at below optimum even after the 21-day period assay.

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 5

### Preparation of Cells Having the Ability to Differentiate into Cells of Osteogenic Lineage by Treatment with Protease and Seeding Cells at High Density in a Differentiation Medium with or without Additional Incubation in Low Serum Medium

In a further example to show that combined action of detaching cells and incubation at high cell density conditions confers plasticity of on already differentiated cells, primary fibroblasts are incubated in the presence of a protease such as trypsin and at high cell density conditions directly in high density plating media preferably before adherence of the cells with optional incubation of cells in a low serum medium. The cells produced by this method are then differentiate into cells of osteogenic lineage, as determined by expression of the osteogenic marker alkaline phosphatase ALP.

Production of Cells Capable of Differentiating into Other Cell Types

Fresh human dermal fibroblasts derived from adult skin or from foreskin are incubated in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells is used as control cells, and the second set of cells is used for testing the capability of cells produced by the method to differentiate into cells of osteogenic lineage. All cells are plated at about 20,000 cells per well/plate or about 740.74 cells per mm² surface area of the well/plate. Once all cells are attached and reach sub-confluence or confluence, the cells are detached as described below.

Alternatively the cells are washed with PBS and then the medium is replaced with DMEM-HG (e.g., Lonza Cat #12-604) or M199 supplemented with 10% FBS for different periods of incubation time, from 10 to 14 days. Optionally, for additional low serum incubation step, cells are washed with PBS and then the medium is replaced with M199 supplemented with 0-1% FBS (low-serum) for different periods of incubation time, from 5 to 9 days.

At the conclusion of the incubation period in the culture medium, test cells are detached by the addition of detachment solution containing 0.12% trypsin, as described in Examples 2 to 4. No trypsin control cells are not detached, and are used directly in the differentiation assay as described below.

Cells are recovered from culture and seeded at concentrations of about 100,000 cells per well/plate or at about 3703.7 cells per mm² surface area of the well/plate before attachment of the cells to the plate/well directly in 100 μl high density plating medium (e.g., +DEX: DMEM-low glucose containing 10% FBS, 20 μg/ml ascorbic acid phosphate-magnesium salt, 1.5 mg/ml beta glycerophosphate and 40 ng/ml dexamethasone) for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation. Cells are then transferred to a complete osteogenic media (+DEX: DMEM-low glucose containing 10% FBS, 20 μg/ml ascorbic acid phosphate-magnesium salt, 1.5 mg/ml beta glycerophosphate and 40 ng/ml dexamethasone) and allowed to expand for about 10-21 days.

As a negative control for the production of progenitor cells, trypsinized cells are seeded at a reduced density i.e., about 740.1 cells per mm² surface area, in high density plating medium (e.g., +DEX: DMEM-low glucose containing 10% FBS, 20 μg/ml ascorbic acid phosphate-magnesium salt, 1.5 mg/ml beta glycerophosphate and 40 ng/ml dexamethasone) and incubated as for samples seeded at high density e.g., for up to about 24 hours or until adherence is achieved and/or as determined by analysis of cell marker expression and/or by the ability of aliquots of cells to subsequently undergo differentiation. The high density plating medium is then replaced with 200 to 400 μl complete osteogenic media and cells are allowed to expand for about 10-21 days.

As a negative control for differentiation, trypsinized cells are seeded at high density in high density plating medium (e.g., e.g., DMEM-HG supplemented with 10% FBS) and incubated as for test samples seeded at high density e.g., for up to about 24 hours or until adherence is achieved and/or as determined by analysis of cell marker expression and/or by the ability of aliquots of cells to subsequently undergo differentiation. The high density plating medium is then replaced with 200 to 400 μl incomplete osteogenic media (−DEX: DMEM-low glucose containing 10% FBS, 20 μg/ml ascorbic acid phosphate-magnesium salt, 1.5 mg/ml beta glycerophosphate) and cells are allowed to expand for about 10-21 days.

As positive control for differentiation, rat bone marrow stromal/stem cells (rBMSCs) are expanded in DMEM medium containing L-Glutamine and 10% FCS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 200 to 400 p. 1 DMEM-HG containing 10% FCS for up to about 24 hours or until adherence is achieved. The medium is replaced with 200 to 400 μl complete osteogenic media and cells are allowed to expand for about 10-21 days.

Medium is replaced every 2 days for both test cells and negative and positive control cells.

Assessment of Osteogenesis using an Alkaline Phosphatase (ALP) Assay

After incubation for up to 14 days in either complete or incomplete osteogenic media as described above, alkaline phosphatase expression is assessed. The media is removed from cells; cells are washed in phosphate buffered saline and lysed with 40 μl of Passive Lysis Buffer (Promega). The lysate is sonicated. After sonication, the lysate is split into two equal samples of 20 μL each. One sample is placed into a separate 48 well plate, to which 180 μL of Hoescht 33258 in buffer (5 μg/mL in 2M NaCl or 20×SSC) (i.e 1:9 ratio of PLB to Hoescht) is added, and the sample is read at Excitation 350 nm/Emission 460 on Molecular Probes fluorescent scanner. p-Nitrophenyl phosphate (pNPP) 75 μL is added to the remaining sample and incubated for 30 minutes at 37° C. One hundred (100) μl of 2M NaOH is subsequently added which will turn into yellow p-Nitrophenylene anion—pNP. An aliquot of 100 μl is transferred to a 96 well plate for plate reading. The absorbance of pNP (yellow) is read on an optical plate reader at 405 nm. A comparison of +Dex to −Dex controls of Absorbance/ng DNA using a PNPP standard curve is made.

### Assessment of Mineral Deposition

After incubation for 14 days in either complete or incomplete osteogenic media as described above, mineral deposition is assessed. To test for mineral deposition, cells are stained with Von Kossa. A comparison of staining intensity is performed on +Dex differentiated cells to −Dex treated controls.

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 6

### Preparation of Cells Having the Ability to Differentiate into Cells of Chondrogenic Lineage by Treatment with Protease and Seeding Cells at High Density in a Differentiation Medium with or Without Additional Incubation in Low Serum Medium

In a further example to show that combined action of detaching cells and incubation at high cell density conditions confers plasticity of on already differentiated cells, primary fibroblasts are incubated in the presence of a protease such as trypsin and at high cell density conditions directly in high density plating media preferably before adherence of the cells with optional incubation of cells in a low serum medium. The cells produced by this method are then differentiate into chondrocytes, as determined by assessment of chondrocyte morphology.

Production of Cells Capable of Differentiating into Other Cell Types

Fresh human dermal fibroblasts derived from adult skin or from foreskin are incubated in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into cells of chondrogenic lineage. All cells are plated at about 20,000 cells per well/plate or about 740.74 cells per mm² surface area of the well/plate. Once all cells are attached and reach sub-confluence or confluence, the cells are detached as described below.

Alternatively the cells are washed with PBS and then the medium is replaced with DMEM-HG (e.g., Lonza Cat #12-604) or M199 supplemented with 10% FBS for different periods of incubation time, from 10 to 14 days. Optionally, for additional low serum incubation step, cells are washed with PBS and then the medium is replaced with M199 supplemented with 0-1% FBS (low-serum) for different periods of incubation time, from 5 to 9 days.

At the conclusion of the incubation period in the culture medium, test cells are detached by the addition of detachment solution containing 0.12% trypsin, as described in Examples 2 to 4. No trypsin control cells are not detached, and are used directly in the differentiation assay as described below.

The cells are recovered from culture and seeded at concentrations of about 100,000 to 200,000 cells per well/plate or at about 3703.7 to 7406.6 cells per mm² surface area of the well/plate before attachment of the cells to the plate/well directly in 100 μl high density plating medium (e.g., DMEM-HG containing ITS+supplement at a 1 fold concentration (final concentrations of 6.25 μg/ml bovine insulin; 6.25 μg/ml transferrin; 6.25 μg/ml selenous acid; 5.33 μg/ml linoleic acid; 1.25 mg/ml BSA) 50 μg/ml ascorbic acid-2-phosphate, 40 μg/ml L-proline, 100 μg/ml pyruvate, 100 nM dexamethasone, 10 ng/ml TGF-β, and 500 ng/ml BMP-2) for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation. Cells are then transferred to a chondrogenic medium (DMEM-HG containing ITS+supplement at a 1 fold concentration (final concentrations of 6.25 μg/ml bovine insulin; 6.25 μg/ml transferrin; 6.25 μg/ml selenous acid; 5.33 μg/ml linoleic acid; 1.25 mg/ml BSA) 50 μg/ml ascorbic acid-2-phosphate, 40 μg/ml L-proline, 100 μg/ml pyruvate, 100 nM dexamethasone, 10 ng/ml TGF-β, and 500 ng/ml BMP-2) and allowed to expand for about 10-21 days.

As a negative control for the production of progenitor cells, trypsinized cells are seeded at a reduced density i.e., about 740.1 cells per mm² surface area, in high density plating medium (e.g., DMEM-HG containing ITS+supplement at a 1 fold concentration (final concentrations of 6.25 μg/ml bovine insulin; 6.25 μg/ml transferrin; 6.25 μg/ml selenous acid; 5.33 μg/ml linoleic acid; 1.25 mg/ml BSA) 50 μg/ml ascorbic acid-2-phosphate, 40 μg/ml L-proline, 100 μg/ml pyruvate, 100 nM dexamethasone, 10 ng/ml TGF-β, and 500 ng/ml BMP-2) and incubated as for samples seeded at high density e.g., for up to about 24 hours or until adherence is achieved and/or as determined by analysis of cell marker expression and/or by the ability of aliquots of cells to subsequently undergo differentiation. The high density plating medium is then replaced with 100 chondrogenic media and cells are allowed to expand for about 10-21 days.

As a negative control for differentiation, trypsinized cells are seeded at high density in high density plating medium (e.g., e.g., DMEM-HG supplemented with 10% FBS) and incubated as for test samples seeded at high density e.g., for up to about 24 hours or until adherence is achieved and/or as determined by analysis of cell marker expression and/or by the ability of aliquots of cells to subsequently undergo differentiation. The high density plating medium is then replaced with 100 μl DMEM-HG containing 1.25 mg/ml BSA, and cells are allowed to expand for about 10-21 days.

As positive control for differentiation, rat bone marrow stromal/stem cells (rBMSCs) are expanded in DMEM medium containing L-Glutamine and 10% FCS, and allowed to attach and reach sub-confluence or confluence. These cells are then detached by incubation with trypsin as described above, and seeded at concentration of about 100,000 cells per well/plate or at about 3703.7 cells per mm² surface area of the well/plate in 200 to 400 μl DMEM-HG containing 10% FCS for up to about 24 hours or until adherence is achieved. The medium is replaced with 100 μl chondrogenic media and cells are allowed to expand for about 10-21 days.

Medium is replaced every 3 days for both test cells and negative and positive control cells.

### Assessment of Chondrogenesis

After incubation for 14 days in either chondrogenic media or control media as described above, cells are assessed by observation for the appearance of chondrocyte morphology. Analysis of the accumulation of sulfated glycosaminoglycans (GAG) is carried out by measuring the amount of 1,9-dimethylmethylene blue-reactive material in extracts of cells treated with chondrogenic media and compared with extracts of control cells. The 1,9-dimethylmethylene blue assay is performed essentially as described in Sabiston et al, Analytical Biochemistry 149: 543-548 (1985).

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 7

### Preparation of Cells Having the Ability to Differentiate into Haematopoietic Cells by Treatment with Protease and Seeding Cells at High Density in a Differentiation Medium with or without Additional Incubation in Low Serum Medium

In a further example to show that combined action of detaching cells and incubation at high cell density conditions confers plasticity of on already differentiated cells, primary fibroblasts are incubated in the presence of a protease such as trypsin and at high cell density conditions directly in high density plating media preferably before adherence of the cells with optional incubation of cells in a low serum medium. The cells produced by this method are then differentiate into hematopoietic cells, as determined by expression of the hematopoietic marker CD45.

Production of Cells Capable of Differentiating into Other Cell Types

Fresh human dermal fibroblasts derived from adult skin or from foreskin are plated at about 20,000 cells per well/plate or about 740.74 cells per mm² surface area of the well/plate and incubated in DMEM-HG supplemented with 10% FBS and allowed to attached and reach sub-confluence or confluence as described in any one of example 3-6.

Alternatively the cells are washed with PBS and then the medium is replaced with DMEM-HG (e.g., Lonza Cat #12-604) or M199 supplemented with 10% FBS for different periods of incubation time, from 10 to 14 days. Optionally, for additional low serum incubation step, cells are washed with PBS and then the medium is replaced with M199 supplemented with 0-1% FBS (low-serum) for different periods of incubation time, from 5 to 9 days.

At the conclusion of the incubation period in the culture medium, test cells are detached by the addition of detachment solution containing 0.12% trypsin, as described in Examples 2 to 4. No trypsin control cells are not detached, and are used directly in the differentiation assay as described below.

The cells are recovered from culture and seeded at concentrations of about 100,000 cells per well/plate or at about 3703.7 cells per mm² surface area of the well/plate before attachment of the cells to the plate/well directly in high density plating medium (e.g., DMEM supplemented with Granulocyte macrophage colony-stimulating factor (GM-CSF; 50 ng/ml) and stem cell factor (SCF; 50 ng/ml)) for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation. Cells are then transferred to an haematopoietic induction_media (DMEM supplemented with Granulocyte macrophage colony-stimulating factor (GM-CSF; 50 ng/ml) and stem cell factor (SCF; 50 ng/ml)) and allowed to expand for about 10-21 days in a humidified atmosphere of 5% CO₂ in air.

As a negative control for the production of progenitor cells, trypsinized cells are seeded at a reduced density i.e., about 740.1 cells per mm² surface area, in high density plating medium (e.g., DMEM supplemented with Granulocyte macrophage colony-stimulating factor (GM-CSF; 50 ng/ml) and stem cell factor (SCF; 50 ng/ml)) and incubated as for samples seeded at high density e.g., for up to about 24 hours or until adherence is achieved and/or as determined by analysis of cell marker expression and/or by the ability of aliquots of cells to subsequently undergo differentiation. The high density plating medium is then replaced with 200 to 400 μl haematopoietic induction_media and cells are allowed to expand for about 10-21 days in a humidified atmosphere of 5% CO₂ in air.

As a negative control for differentiation, trypsinized cells are seeded at high density in high density plating medium (e.g., e.g., DMEM-HG supplemented with 10% FBS) and incubated as for test samples seeded at high density e.g., for up to about 24 hours or until adherence is achieved and/or as determined by analysis of cell marker expression and/or by the ability of aliquots of cells to subsequently undergo differentiation. The high density plating medium is then replaced with 200 to 400 μl DMEM-HG containing 1.25 mg/ml BSA and cells are allowed to expand for about 10-21 days in a humidified atmosphere of 5% CO₂ in air.

As positive control for differentiation, rat bone marrow stromal/stem cells (rBMSCs) are expanded in DMEM medium containing L-Glutamine and 10% FCS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 200 to 400 μl DMEM-HG containing 10% FCS for up to about 24 hours or until adherence is achieved. The medium is replaced with 200 to 400 μl haematopoietic induction_media (DMEM supplemented with Granulocyte macrophage colony-stimulating factor (GM-CSF; 50 ng/ml) and stem cell factor (SCF; 50 ng/ml)) and cells are allowed to expand for about 10-21 days in a humidified atmosphere of 5% CO₂ in air. Medium is replaced every 3 days for both test cells and negative and positive control cells.

### Assessment of Differentiated Haematopoietic Cells

After incubation for 14 days in haematopoietic media, cells are harvested and analyzed for cells expressing the hematopoietic marker CD45 by flow cytometry. To detect the presence of the cell surface CD45 antigen, cells are incubated for 30 min. at 37° C. with anti-CD45 antibodies (Becton Dickinson), washed in PBS and analysed by flow cytometry. Flow cytometric analysis is performed using a FACSCalibur flow cytometer and the CellQuest software program (Becton Dickinson Immunocytometry Systems, San Jose, Calif.). Data analysis is performed using CellQuest and the Modfit LT V2.0 software program (Verity Software House, Topsham, Me.).

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 8

### Preparation of Cells Having the Ability to Differentiate into Insulin-Secreting Cells by Treatment with Protease and Seeding Cells at High Density in a Differentiation Medium with or without Additional Incubation in Low Serum Medium

In a further example to show that combined action of detaching cells and incubation at high cell density conditions confers plasticity of on already differentiated cells, primary fibroblasts are incubated in the presence of a protease such as trypsin and at high cell density conditions directly in high density plating media preferably before adherence of the cells with optional incubation of cells in a low serum medium. The cells produced by this method are then differentiate into insulin-secreting cells, as determined by formation of islet-like cell clusters and expression of insulin and nestin in re-differentiated cells.

Production of Cells Capable of Differentiating into Other Cell Types

Fresh human dermal fibroblasts derived from adult skin or from foreskin are plated at about 20,000 cells per well/plate or about 740.74 cells per mm² surface area of the well/plate and incubated in DMEM-HG supplemented with 10% FBS and allowed to attached and reach sub-confluence or confluence as described in any one of example 2-6.

Alternatively the cells are washed with PBS and then the medium is replaced with DMEM-HG (e.g., Lonza Cat #12-604) or M199 supplemented with 10% FBS for different periods of incubation time, from 10 to 14 days. Optionally, for additional low serum incubation step, cells are washed with PBS and then the medium is replaced with M199 supplemented with 0-1% FBS (low-serum) for different periods of incubation time, from 5 to 9 days. At the conclusion of the incubation period in the culture medium, test cells are detached by the addition of detachment solution containing 0.12% trypsin, as described in Examples 2 to 4. No trypsin control cells are not detached, and are used directly in the differentiation assay as described below.

Test cells are recovered from culture and seeded at concentrations of about 100,000 cells per well/plate or at about 3703.7 cells per mm² surface area of the well/plate before attachment of the cells to the plate/well directly in 200 to 400 μl high density plating medium (e.g., DMEM-HG supplemented with 10% FBS) for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation.

Adherent cells are then transferred at high density to DMEM serum free medium to enrich for nestin-positive cells (see Lumelsky et al., Science, 292:1389, 2001) for 2-3 days. The nestin-positive cells are then sub-subcultured and expanded for 6 to 7 days in serum-free N2 media supplemented with 1 μg/ml laminin, 10 ng/ml bFGF, 500 ng/ml N-terminal fragment of murine or human SHH (sonic hedge hog) 100 ng/ml FGF8 and B27 media supplement, as described in Lee et al. Nature Biotechnology, 18: 675 (2000) and Lumelsky (supra), which are herein incorporated by reference. After the nestin-positive cells are expanded, the growth factors (FGF, SHH) are removed from the media and nicotinamide is added to the media at a final concentration of 10 mM, to promote the cessation of cell proliferation and induce the differentiation of insulin-secreting cells.

### Assessment of Insulin-Secreted Cells

After approximately 6 days of growth factor starvation, aggregates of insulin-secreting cells are formed (islet-like cell clusters). Differentiated cells are observed under inverse microscopy, insulin and nestin expression in differentiated cells are detected with immunocytocehmistry.

Insulin excreted from differentiated cells are tested with radioimmunoassay (RIA) (see Li-Bo Chen et al., World J Gastroenterol 2004; 10(20):3016-3020).

For immunohistochemistry, adherent cells are fixed to slides with 40 g/L para-formaldehyde. Cells are washed and incubated with biotin-goat anti-rat insulin or nestin monoclonal antiobodies (Santa Cruz Co, USA) diluted 1:200 in 50 mL/L normal goat serum for 20 min at room temperature. Immuno-reactive cells are visualized using diaminobenzidine tetrachloride (DAB) (Boehringer-Mannheim) as the chromogen. All sections are counterstained with hematoxylin.

Radioimmunoassay (RIA), immunoreactive insulin in supernatants secreted from test and control cells are determined using a commercially available RIA kit according to manufacturer's instructions Millipore (CHEMICON/Upstate/Linco). Briefly, to each polypropylene RIA tube 100 μl of each anti-insulin, ¹²⁵I-insulin, and insulin or the cell supernatant samples are added. Immunocomplexes are precipitated 24 h later with 1 ml of 160 ml/L polyethylene glycol solution, and gamma counter is used to determine radioactivity in the precipitates.

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 9

### Preparation of Cells Having the Ability to Differentiate into Dopamine-Secreting Neuronal Cells by Treatment with Protease and Seeding Cells at High Density in a Differentiation Medium with or Without Additional Incubation in Low Serum Medium

In a further example to show that combined action of detaching cells and incubation at high cell density conditions confers plasticity of on already differentiated cells, primary fibroblasts are incubated in the presence of a protease such as trypsin and at high cell density conditions directly in high density plating media preferably before adherence of the cells with optional incubation of cells in a low serum medium. The cells produced by this method are then differentiate into neuronal cells, as determined by dopamine synthesis.

Production of Cells Capable of Differentiating into Other Cell Types

Fresh human dermal fibroblasts derived from adult skin or from foreskin are plated at about 20,000 cells per well/plate or about 740.74 cells per mm² surface area of the well/plate and incubated in DMEM-HG supplemented with 10% FBS and allowed to attached and reach sub-confluence or confluence as described in any one of example 2-6.

Alternatively the cells are washed with PBS and the medium is replaced with DMEM-HG (e.g., Lonza Cat #12-604) or M199 supplemented with 10% FBS for further incubation period from 10 to 14 days. Optionally for low serum incubation step, cells are washed with PBS and then the medium is replaced with M199 supplemented with 0-1% FBS (low-serum) for different periods of incubation time, from 5 to 9 days.

At the conclusion of the incubation period in the culture medium, test cells are detached by the addition of detachment solution containing 0.12% trypsin, as described in Examples 2 to 3. No trypsin control cells are not detached, and are used directly in the differentiation assay described below.

To produce progenitor cells, test cells are recovered from culture and seeded at concentrations of about 200,000 cells per well/plate or at about 7407.4 cells per mm² surface area of the well/plate before attachment of the cells to the plate/well directly in 200 to 400 μl high density plating medium (e.g., DMEM-HG supplemented with 10% FBS) for a time and under conditions sufficient for an optimum number of progenitor cells to be produced e.g., for up to about 24 hours or until adherence is achieved, i.e., a shorter time than required for cells to become adherent and/or as determined by analysis of cell marker expression and/or by the ability of aliquots of cells to subsequently undergo differentiation.

To produce dopamine-secreting neuronal cells, adherent cells are then transferred at high density to dopaminergic induction media (DMEM serum free medium supplemented with 2 mM glutamine, 100 μg/ml streptomycin, 100 U/ml penicillin, 12.5 U/ml nystatin, N2 supplement (Invitrogen, New Haven, Conn.), and 20 ng/ml fibroblast growth factor-2 (FGF-2) and epidermal growth factor (EGF) (both from R&D Systems, Minneapolis, Minn.) for 2-3 days. The medium is then changed to 200 to 400 μl basic induction medium containing Neurobasal and B27 (both from Invitrogen), in addition to 1 mM dibutyryl cyclic AMP (db cAMP), 3-isobutyl-1-methylxanthine (IBMX), and 200 μM ascorbic acid (all from Sigma, St Louis, Mo.) and brain-derived neurotrophic factor (BNDF) 50 ng/ml (Cytolab, Rehovot, Israel), for 5 to 7 days as described in Barzilay et al., Stem cells and Development 17:547-554, 2008 which is herein incorporated by reference. Media is replaced every on day 2 and day 5 days.

### Assessment of Dopaminergic Neuronal Cells

Differentiated cells are detected with immunocytocehmistry and/or intracellular staining and fluorescence-activated cell sorter (FACS) analysis of expression levels of tyrosine hydroxylase (TH), the rate-limiting enzyme in dopamine synthesis.

For immunocytochemistry, cells are fixed with 4% paraformaldehyde, blocked, and permiabilized in 5% goat serum (Biological Inducstries), 1% bovine serum albumin (BSA; Sigma), and 0.5% Triton-X in PBS for 1 h at room temperature. Primary antibodies include mouse anti-TH and mouse anti-β3-tubulin (both 1:1,000; Sigma), followed by goat anti-mouse Alexa-488 or Alexa-568 (both 1:1,000; Molecular probes). DNA-specific fluoresent dye 4′,6-diamidino-2-phenylindole (DAPI; Sigma) counter stain is used to detect cell nuclei. Cells are photographed with fluorescence Olympus IX70-S8F2 microscope with a fluorescent light source (excitation wavelength, 330-385 nm, barrier filter, 420 nm) and a U-MNU filter tube (Olympus, Center Valley, Pa.).

For intracellular FACS analysis, test and control cells are harvested from the tissue culture plates, centrifuged, and resuspended in PBS. Intracellular staining is performed with IntraCyte kit (Orion Biosolutions, Vista, Calif.), according to manufacturer's instructions. TH staining is performed with mouse anti-TH antibody (1:1,000; Sigma) followed by donkey anti-mouse phycoerythrin (PE)-conjugated immunoglobulin G (IgG; Jackson Laboratories, Bar Harbor, Me.). The results are analysed with CellQuest software. A PE-conjugated isotype control is included in each experiment. To verify specific detection of TH expression, HeLa cells are employed as a negative control and PC12 (ATCC) cells as a positive control.

For example, production of progenitor cells method described herein, may be enhanced by agonism of the Akt/(PKB) and/or the NF-κB pathway by incubation of the differentiated cells in the presence of an agonist compound as described in any one of the examples 10 to 21.

### Example 10

### Preparation of Cells Having the Ability to Differentiate into Other Cell Types by Induction of the Akt/(PKB) Pathway: Method 1

The data in Examples 1-9 suggested to the inventor that agonism of the Akt/(PKB) pathway and/or NF-κB pathway may produce equivalent or improved results as the combined action of detaching cells for example by incubation in the presence of a protease such as trypsin and incubation at high cell density conditions directly in high density plating media before preferably adherence of the cells. Without being bound by any theory or mode of action, the inventor reasoned that the detachment of the cells and high density culture, maintenance and incubation to induce optimum plasticity of fibroblasts coincided with the induction and/or enhancement of the Akt/(PKB) pathway, and that the responses of cells to the combined detachment of cells e.g., by trypsinization conditions and high cell density culture, maintenance and incubation conditions is likely to induce the Akt/(PKB) pathway. Accordingly, the inventor sought to test whether or not the effect of incubation in the presence of a protease such as trypsin and high density culture, maintenance and incubation conditions could be reproduced or improved upon by incubation in the presence of one or more agonists of the Akt/(PKB) pathway. An advantage of using an agonist to induce the Akt/(PKB) pathway, as opposed to detaching cells e.g., by trypsinization followed by high density incubation, or in concert with such a process, is enhancement of the proportions of cells achieving optimum plasticity. By enhancing induction of the Akt/(PKB) pathway using an agonist compound, differentiated primary cells and cell lines that would normally enter a quiescent state or undergo apoptosis under high cell density incubation conditions can be used to produce cells capable of differentiating into different cell types.

In one example to show that Akt/(PKB) pathway induction confers or enhances plasticity of fibroblasts, primary human foreskin fibroblasts are incubated in the presence of human recombinant PDGF-BB for a time and under conditions sufficient to induce the Akt/(PKB) pathway.

Production of Cells Capable of Differentiating into Different Cell Types

Fresh human dermal fibroblasts that are derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA) (low-protein) for 24 hours. After 24 hours, the medium is replaced with low-serum/BSA or BSA/serum-free DMEM containing 10 to 100 ng/ml of human recombinant PDGF-BB (Invitrogen) for 5 to 15 min to activate the Akt/(PKB) pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the treated fibroblasts into other cell types is achieved by reseeding the treated cells described above into a suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 11

### Preparation of Cells Having the Ability to Differentiate into Other Cell Types by Induction of the Akt/(PKB) Pathway: Method 2

In a further example to show that Akt/(PKB) pathway induction confers or enhances plasticity of fibroblasts, primary fibroblasts are incubated in the presence of TGF-β for a time and under conditions sufficient to induce the Akt/(PKB) pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA) (low-protein) for 24 hours to precondition the cells to TGF-β. After 24 hours, the medium is replaced with BSA or serum-free or low-serum or BSA DMEM-HG (e.g., Lonza) containing 1 to 10 ng/ml of TGF-β (R&D systems) for at least 60 min to activate the Akt/(PKB) pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the treated fibroblasts into other cell types is achieved by reseeding the treated cells described above in suitable differentiation media. Methods suitable for differentiation of these cells into adipocytes, cells of osteogenic lineage, chondrogenic lineage, haematopoietic cells insulin secreting cells, or dopamine-secreting cells are known in the art and described herein e.g., Examples 2 to 9.

### Example 12

### Preparation of Cells Having the Ability to Differentiate into Other Cell Types by Induction of the Akt/(PKB) Pathway: Method 3

In a further example to show that Akt/(PKB) pathway induction confers or enhances plasticity of fibroblasts, primary fibroblasts are incubated in the presence of sodium pyruvate for a time and under conditions sufficient to induce the Akt/(PKB) pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or BSA (low-protein) for 24 hours to pre-condition the cells to sodium pyruvate. After 24 hours, the medium is replaced with serum-free or low-serum DMEM-HG containing 50 to 200 mg/L of cell culture grade sodium pyruvate (e.g., Lonza), and preferably, at 110 mg/L for at least 1 h to activate the Akt/(PKB) pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the treated fibroblasts into other cell types is achieved by reseeding the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 13

### Preparation of Cells Having the Ability to Differentiate into Other Cell Types by Induction of the Akt/(PKB) Pathway: Method 4

In a further example to show that Akt/(PKB) pathway induction confers or enhances plasticity of fibroblasts, mouse dermal primary fibroblasts are incubated in the presence of PDGF-BB for a time and under conditions sufficient to induce the Akt/(PKB) pathway.

Production of Cells Capable of Differentiating into Different Cell Types

Mouse dermal fibroblast cells are prepared from 8 to 12 week-old C57BL/6 mice. Briefly, mice are anesthetized with pentobarbital (50 mg/kg body weight), and a full thickness of the back skin is cut out by scissors. The skin tissues are cut into small pieces and are implanted into plastic tissue culture dishes containing DMEM-HG (e.g., Lonza) with 10% FBS. The fibroblast cultures are used after three to seven passages.

Adherent fibroblast cultures are incubated in DMEM-HG supplemented with 0-1% FBS or BSA (low-protein) for 48 hours to precondition the cells to PDGF-BB. After 48 hours, the medium is replaced with BSA or serum-free DMEM-HG or low-serum or BSA DMEM-HG containing 10 to 100 ng/ml of human recombinant PDGF-BB (Invitrogen) for 15 to 60 min to activate the Akt/(PKB)/(PKB) pathway.

Preferably, treated cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the treated fibroblasts into other cell types is achieved by the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 14

### Preparation of Cells Having the Ability to Differentiate into Other Cell Types by Induction of the Akt/(PKB) Pathway: Method 5

In a further example to show that Akt/(PKB) pathway induction confers or enhances plasticity of cells generally, rat adrenal cells are incubated in the presence of Carbachol or NGF for a time and under conditions sufficient to induce the Akt/(PKB) pathway.

Production of Cells Capable of Differentiating into Different Cell Types

PC12 cells are obtained from the American Type Culture Collection (CRL-1721, Rockville, Md.). PC12 cells are cultured in DMEM-HG supplemented with 5% (v/v) fetal calf serum and 10% (v/v) heat-inactivated horse serum, and grown at 37° C. in an environment of 7.5% CO₂ as described previously (Yu et al, Neurosignals 13: p248 (2004).

Adherent PC12 cultures are incubated in DMEM supplemented with 0-1% FBS or BSA (low-protein) for 24 hours to precondition the cells to Carbachol or NGF. After 24 hours, the medium is replaced with BSA or serum-free DMEM-HG or low-serum or BSA DMEM containing 200-1000 μM Carbachol (Calbiochem) or at least 50 ng/ml purified NGF (2.5S) (Alomone Labs Ltd) for 5 to 10 min to activate the Akt/(PKB) pathway.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Re-differentiation of the cell product into other cell types is achieved by reseeding the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 15

### Preparation of Cells Having the Ability to Differentiate into Other Cell Types by Induction of the Akt/(PKB) Pathway: Method 6

In a further example to show that Akt/(PKB) pathway induction confers or enhances plasticity of cells generally, embryo fibroblasts are incubated in the presence of insulin growth factor-1 (IGF-1) for a time and under conditions sufficient to induce the Akt/(PKB) pathway.

Production of Cells Capable of Differentiating into Different Cell Types

Non-transformed rat embryo fibroblasts (Rat-1) are prepared and maintained as previously described (Peterson, et al., J. Biol. Chem. 271:31562-31571 (1996)).

Adherent Rat-1 cultures are incubated in DMEM-HG supplemented with 0-1% FBS or BSA (low-protein) for 12 hours. After 12 hours, the medium is replaced with BSA or serum-free DMEM or low-serum or BSA DMEM containing at least 250 ng/ml of insulin growth factor-1 (IGF-1; Sigma) for at least about 20 min to activate the Akt/(PKB) pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Re-differentiation of the cell product into other cell types is achieved by reseeding the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 1 to 8.

### Example 16

### Preparation of Cells Having the Ability to Differentiate into Other Cell Types by Induction of the NF-κB Pathway: Method 1

The data in examples 1 to 9 also suggested to the inventor that agonism of the NF-κB pathway may produce equivalent or improved results as the combined action of detaching cells for example by incubation in the presence of a protease such as trypsin and incubation at high cell density conditions directly high density plating media capable of supporting differentiation of progenitor cells preferably before adherence of the cells. Without being bound by any theory or mode of action, the inventor reasoned that the detachment of the cells and high density culture, maintenance and incubation conditions to induce optimum plasticity of fibroblasts coincided with the induction and/or enhancement of the NF-κB pathway, and that the responses of cells to the combined detachment of the cells e.g., by trypsinization conditions and high cell density culture, maintenance and incubation conditions is likely to induce the NF-κB pathway. Accordingly, the inventor sought to test whether or not the effect of incubation in the presence of a protease such as trypsin and high density incubation could be reproduced or improved upon by incubation in the presence of one or more agonists of the NF-κB pathway. An advantage of using an agonist to induce the NF-κB pathway, in concert with as opposed to detaching cells e.g., by trypsinization followed by incubation at high cell density conditions in a high density plating medium, is the enhancement in the proportion of cells achieving optimum plasticity. By enhancing induction of the NF-κB pathway using an agonist compound, differentiated primary cells and cell lines that would normally enter a quiescent state or undergo apoptosis under high density culture, maintenance and incubation conditions can be used to produce cells capable of differentiating into different cell types.

In one example to show that NF-κB pathway induction confers or enhances plasticity of cells generally, primary human dermal fibroblasts are incubated in the presence of TNF-α for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with serum-free DMEM or low-serum DMEM-HG containing at least 20 ng/ml of TNF-α (Roche) for at least 60 min to activate the NF-κB pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Re-differentiation of the cell product into other cell types is achieved by reseeding the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 17

### Preparation of Cells Having the Ability to Differentiate into Other Cell Types by Induction of the NF-κB Pathway: Method 2

In a further example to show that NF-κB pathway induction confers or enhances plasticity of cells generally, primary human dermal fibroblasts are incubated in the presence of interleukin-1α for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with DMEM supplemented with 0.25% FBS for 50 hours to precondition the cells to interleukin-1α. After 50 hours, the cells are treated with recombinant human IL-1α at a concentration of least 0.27 ng/ml to activate the NF-κB pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the cell product into other cell types is achieved by the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 18

### Preparation of Cells Having the Ability to Differentiate into Other Cell Types by Induction of the NF-κB Pathway: Method 3

In a further example to show that NF-κB pathway induction confers or enhances plasticity of fibroblasts, primary fibroblasts are incubated in the presence of sodium pyruvate for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or BSA (low-protein) for 24 hours to precondition the cells to pyruvate. After 24 hours, the medium is replaced with BSA or serum-free or low-serum or BSA DMEM-HG with containing 50 to 200 mg/L of cell culture grade sodium pyruvate (e.g., Lonza), and preferably, at 110 mg/L for at least 1 h to activate the NF-κB pathway. Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the cell product into other cell types is achieved by reseeding the treated cells described above in into differentiation media for adipocytes, or as described herein e.g., Examples 1, 2 and 81 to 84.

### Example 19

### Preparation of Cells Having the Ability to Differentiate into Other Cell Types by Induction of the NF-κB Pathway: Method 4

In a further example to show that NF-κB pathway induction confers or enhances plasticity of cells generally, mouse embryo fibroblasts are incubated in the presence of L-alpha-Lysophosphatidic acid (C18:1, [cis]-9), LPA for a time and under conditions sufficient to induce the NF-κB pathway.

Production of Cells Capable of Differentiating into Different Cell Types

Swiss 3T3 mouse embryo fibroblasts are obtained from the American Type Culture Collection (CCL-92, Rockville, Md.) and are cultured at 37 C under a humidified atmosphere of 10% CO₂ in Dulbecco's modified Eagle's medium (DMEM) containing 10% (v/v) fetal calf serum.

Adherent 3T3 fibroblast cultures are incubated in DMEM-HG supplemented with 1% FBS (low-serum) for 18 hours. After 18 hours, L-α-Lysophosphatidic acid (C18:1,[cis]-9), LPA (Calbiochem; prepared as a stock of 1 mg/ml in phosphate-buffered saline containing 10 mg/ml essentially fatty acid-free bovine serum albumin (Sigma) is added to adherent cultures at 40-100 μM final concentration for about 40-120 min to activate the NF-κB pathway. As a control, TNF-α (Roche) is added to separate parallel cultures at a final concentration of 30 ng/ml for the same time period to activate the NF-κB pathway e.g., as described in Example 14.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the cell product into other cell types is achieved by reseeding the treated cells described above in suitable differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 2 to 9.

### Example 20

### Preparation of Cells Having the Ability to Differentiate into Other Cell Types by Induction of the NF-κB Pathway: Method 5

In a further example to show that NF-κB pathway induction confers or enhances plasticity of cells generally, human myometrial microvascular endothelial cells (HUMEC) are incubated in the presence of Lipopolysaccharide (LPS) for a time and under conditions sufficient to induce the NF-κB pathway.

Production of Cells Capable of Differentiating into Different Cell Types

Human myometrial microvascular endothelial cells (HUMEC) are obtained from Technoclone GmbH (Vienna, Austria) and are cultured at 37 C in endothelial growth medium according to the specifications supplied by Technoclone GmbH.

Adherent HUMEC cultures are then incubated in endothelial medium, preferably serum free or containing low-serum concentration, and supplemented with 10-100 ng/ml of Lipopolysaccharide (LPS; Sigma) for at least 45 min to activate the NF-κB pathway. Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the cell product into other cell types is achieved by reseeding the treated cells described above in differentiation media. Methods suitable for differentiation of these cells into adipocytes, cells of osteogenic lineage, chondrogenic lineage, haematopoietic cells, insulin secreting cells or dopamine-secreting cells are known in the art and described herein e.g., Examples 1, 2 and 81 to 84.

### Example 21

### Preparation of Cells Having the Ability to Differentiate into Other Cell Types by Induction of the NF-κB Pathway: Method 6

In a further example to show that NF-κB pathway induction confers or enhances plasticity of cells generally, synovial fibroblasts are incubated in the presence of Lipopolysaccharide (LPS) for a time and under conditions sufficient to induce the NF-κB pathway.

Production of Cells Capable of Differentiating into Different Cell Types

Primary cultures of synovial fibroblasts are obtained and maintained in culture as described previously (Brinckerhoff, and Mitchell, Journal of Cellular Physiology, 136 (1):72-80 (2005)).

Adherent synovial fibroblast cultures are then incubated in growth medium, preferably serum free or containing low-serum concentration, and supplemented with 10-100 ng/ml of Lipopolysaccharide (LPS; Sigma) for at least 45 min to activate the NF-κB pathway.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, preferably within about 4 to 6 hours after trypsinization and are then diluted to about 100,000 to 200,000 cells in 100 μl in a high density plating medium. Cells are then seeded directly in the high density plating medium at high cell density of about 100,000 cells to 200,000 cells per well/plate (i.e., 100 to 200 μl per well/plate) or at about 3703.7 to 7407.4 cells per mm² surface area of the well/plate before adherence of the cells to the well/plates and maintained under high density conditions in the high density plating medium until required for re-differentiation.

Differentiation into Other Cell Types

Re-differentiation of the cell product into other cell types is achieved by reseeding the treated cells described above into differentiation media. Methods suitable for differentiation of these cells into adipocytes, cells of osteogenic lineage, chondrogenic lineage, haematopoietic cells or insulin secreting cells or dopamine-secreting cells are known in the art and described herein e.g., Examples 1, 2 and 81 to 84.

### Example 22

### Preparation of Cells Having the Ability to Differentiate into Other Cell Types by Incubation in Medium Containing a Modulator of 5AMP-Activated Protein Kinase or AMPK and Treatment with Protease: Method 1

In a this set of experiments for producing cells having the ability to differentiate into different cell types, fibroblasts are cultured and then either incubated in media with or without AICAR [5-aminoimidazole-4-carboxamide-1-β-4-ribofuranoside] as a modulator of 5′AMP-activated protein kinase or AMPK and then are either incubated in media without trypsin or containing trypsin.

The cells produced by this method are then tested for their ability to differentiate into adipocytes, as determined by the accumulation of fat. Differentiation into adipocytes is selected in these primary experiments because methods for such differentiation are well-established.

### 1.1 Materials and Methods

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium Low Glucose (DMEM-LG) containing 0-3 mM glucose supplemented with about 0.5 mM to about 1.0 mM AICAR [5-aminoimidazole-4-carboxamide-1-β-4-ribofuranoside] (purchased from Cell Signalling Technology, Beverly, Mass., USA) for 24 hours. Alternatively, once all cells are attached, the medium is replaced with growth medium Dulbecco's Modified Eagle Medium High Glucose (DMEM-HG) supplemented with about 2 mM AICAR for 60 minutes. Control cells are incubated with the same medium as test cells without AICAR. Without being bound by theory, the inventor reasoned that AICAR is a cell permeable drug that is converted to AICAR monophosphate (ZMP) intracellulary and that mimics the stimulatory effect of AMP on AMPK.

At the conclusion of the incubation period in media containing AICAR, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells described above into differentiation media for adipocytes, or as described herein e.g., Examples 1, 2 and 81 to 84.

### Example 23

### Preparation of Cells Having the Ability to Differentiate into Other Cell Types by Incubation in Medium Containing a Modulator of 5′AMP-Activated Protein Kinase or AMPK and Treatment with Protease: Method 2

In a this set of experiments for producing cells having the ability to differentiate into different cell types, fibroblasts are cultured and then either incubated in media with or without Metformin (Glucophage) as a modulator of 5′AMP-activated protein kinase or AMPK and then are either incubated in media without trypsin or containing trypsin. The cells produced by this method are then tested for their ability to differentiate into adipocytes, as determined by the accumulation of fat.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium Low Glucose (DMEM-LG) containing 0-3 mM glucose supplemented with about 2 mM Metformin (purchased from Sigma Chemical Co., St Louis, Mo., USA) for 18 hours. Control cells are incubated with the same medium as test cells without Metformin.

At the conclusion of the incubation period in media containing Metformin, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Control cells are not detached, and are used directly in the differentiation assay as described in Example 2. Differentiation into Adipocytes and assessment of adipogenesis is carried out as described in Example 2.

For example, differentiation of the cell product into other cell types is achieved by reseeding the cells described in Example 1 into differentiation media for adipocytes, or as described herein e.g., Examples 1, 2 and 81 to 84.

### Example 24

### Preparation of Cells Having the Ability to Differentiate into Other Cell Types by Incubation in Medium Containing a Modulator of 5′AMP-Activated Protein Kinase or AMPK and Treatment with Protease: Method 3

In a this set of experiments for producing cells having the ability to differentiate into different cell types, fibroblasts are cultured and then either incubated in media with or without Compound C (6-[4-(2-Piperidin-1-yl-ethoxy)-phenyl)]-3-pyridin-4-yl-pyrrazolo[1,5-a]-pyrimidine) as a modulator of 5′AMP-activated protein kinase or AMPK and then are either incubated in media without trypsin or containing trypsin. The cells produced by this method are then tested for their ability to differentiate into adipocytes, as determined by the accumulation of fat.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium Low Glucose (DMEM-LG) containing 0-3 mM glucose supplemented with about 10 μM Compound C (purchased from Calbiochem, San Diego, Calif., USA) for 18 hours or 20 mM of Compound C for 60 minutes. Control cells are incubated with the same medium as test cells without Compound C. Without being bound by theory, the inventor reasoned that compound C induces AMPK inhibition in cells and since AMPK is a modulator of glycolysis, AMPK inhibition by Compound C in cells further reduces cellular glycolysis under low glucose conditions.

At the conclusion of the incubation period in media containing Metformin, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Control cells are not detached, and are used directly in the differentiation assay as described in Example 2. Differentiation into Adipocytes and assessment of adipogenesis is carried out as described in Example 2.

For example, differentiation of the cell product into other cell types is achieved by reseeding the cells described in Example 1 into differentiation media for adipocytes, or as described herein e.g., Examples 1, 2 and 81 to 84.

### Example 25

### Preparation of Cells Having the Ability to Differentiate into Other Cell Types by Incubation in Medium Containing a Modulator of 5′AMP-Activated Protein Kinase or AMPK and Treatment with Protease: Method 4

In a this set of experiments for producing cells having the ability to differentiate into different cell types, fibroblasts are cultured and then either incubated in media with or without Thrombin as a modulator of 5′AMP-activated protein kinase or AMPK and then are either incubated in media without trypsin or containing trypsin. The cells produced by this method are then tested for their ability to differentiate into adipocytes, as determined by the accumulation of fat.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) comprising 10 mM glucose supplemented with about 2 U/ml Thrombin (Sigma) for 15 min Control cells are incubated with the same medium as test cells without Thrombin.

At the conclusion of the incubation period in media containing Thrombin, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 with DMEM-HG (e.g., Lonza, Cat #12-604) (with 10% FBS) and maintained in this medium until required for re-differentiation. Control cells are not detached, and are used directly in the differentiation assay as described in Example 2. Differentiation into Adipocytes and assessment of adipogenesis is carried out as described in Example 2.

For example, differentiation of the cell product into other cell types is achieved by reseeding the cells described in Example 1 into differentiation media for adipocytes, or as described herein e.g., Examples 1, 2 and 81 to 84.

### Example 26

### Preparation of Cells Having the Ability to Differentiate into Other Cell Types by Incubation in Medium Containing a Modulator of 5′AMP-Activated Protein Kinase or AMPK and Treatment with Protease: Method 5

In a this set of experiments for producing cells having the ability to differentiate into different cell types, fibroblasts are cultured and then either incubated in media with or without Ghrelin, an orexigenic hormone, as a modulator of 5′AMP-activated protein kinase or AMPK and then are either incubated in media without trypsin or containing trypsin. The cells produced by this method are then tested for their ability to differentiate into adipocytes, as determined by the accumulation of fat.

Production of Cells Capable of Differentiating into Other Cell Types

Fresh human dermal fibroblasts derived from adult skin or from foreskin are purchased from PromoCell®. Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) supplemented with about 10⁻⁶ M Ghrelin for 60 min or 10⁻⁷ M Ghrelin for 90 min or with 10⁻⁹ M Ghrelin for 6 hours. Ghrelin is purchased from Peptide Institute (Osaka, Japan). Control cells are incubated with the same medium as test cells without Ghrelin.

At the conclusion of the incubation period in media containing Ghrelin, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Control cells are not detached, and are used directly in the differentiation assay as described in Example 2. Differentiation into Adipocytes and assessment of adipogenesis is carried out as described in Example 2.

For example, differentiation of the cell product into other cell types is achieved by reseeding the cells described in Example 1 into differentiation media for adipocytes, or as described herein e.g., Examples 1, 2 and 81 to 84.

### Example 27

### Preparation of Cells Having the Ability to Differentiate into Other Cell Types by Incubation in Medium Containing a Modulator of 5′AMP-Activated Protein Kinase or AMPK and Treatment with Protease, with Additional Incubation in Low-Serum

The inventor sought to test whether or not the additional step of incubating cells in a low serum media may produce equivalent or improved results as the combined action of incubation with a modulator of 5′AMP-activated protein kinase or AMPK and incubation with a protease or alternatively by agonism of the Akt/(PKB) and/or the NF-κB pathway using an agonist compound. Without being bound by any theory or mode of action, the inventor reasoned that low-serum incubation conditions for 5-9 days further induces/enhances activation of the Akt/(PKB) and/or the NF-κB pathway. A possible advantage of using low-serum incubation for 5-9 days in concert with incubation with a modulator of 5′AMP-activated protein kinase or AMPK and detachment of cells e.g., by incubation with a protease such as trypsin and/or inducing the Akt/(PKB) and/or the NF-κB pathway using an agonist compound, is an increase in proportion of cells achieving optimum plasticity.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per mm² plating surface area of the vessel. Once all cells are attached, the medium is replaced with medium 199 (M199) (e.g., Sigma) supplemented with 0-1% FBS (low-serum) for different periods of time, from 1 to 11 days.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 1. Control cells are incubated with the same medium as test cells without AICAR. Alternatively, the medium is replaced with DMEM-LG containing 0-3 mM glucose supplemented with 2 mM Metformin for 18 hours, and incubated as described in example 2.

Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 3. Control cells are incubated with the same medium as test cells without Compound C. Alternatively, the medium is replaced with DMEM-HG containing supplemented with 2 U/ml Thrombin for 15 min, and incubated as described in Example 4. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 6. Control cells are incubated with the same medium as test cells without Ghrelin.

At the conclusion of the incubation period in low serum media, test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with serum-free DMEM-HG (e.g., Lonza, Cat #12-604) (0% FBS) and maintained in serum-free medium until required for re-differentiation. Control cells are not detached, and are used directly in the differentiation assay as described in Example 2. Differentiation into Adipocytes and assessment of adipogenesis is carried out as described in Example 2. The person skilled in the art would appreciate that differentiation of test cells into adipocytes may continue albeit at below optimum even after the 11-day period incubation at low serum.

For example, differentiation of the cell product into other cell types is achieved by reseeding the cells described in Example 1 into differentiation media for adipocytes, or as described herein e.g., Examples 1, 2 and 81 to 84.

### Example 28

### Preparation of Cells Having the Ability to Differentiate into Other Cell Types by Incubation with a Modulator of 5′AMP-Activated Protein Kinase or AMPK and Treatment with Protease and Additional Incubation at High Cell Density Conditions

To improve the yield of cells having the ability to differentiate into other cell types, the inventor sought to investigate the effect of high density cultures on plasticity. Specifically, the inventor sought to test whether or not the additional step of incubating cells at high density in a high density plating medium capable of supporting progenitor cells may produce equivalent or improved results as the combined action of incubation with a modulator of AMPK and incubation in the presence of protease or alternatively by agonism of the Akt/(PKB) and/or the NF-κB pathway using an agonist compound. Without being bound by any theory or mode of action, the inventor reasoned that culturing protease treated cells at high cell density in high density plating medium, further induces activation of the NF-κB pathway, possibly by inducing the intracellular PKC or Ca2+ influx. A possible advantage of using a high cell density culturing, maintenance or incubation following protease treatment to induce the NF-κB pathway, in concert with incubating cells with a modulator of AMPK and in the presence of a protease and/or inducing the Akt/(PKB) and/or the NF-κB pathway using an agonist compound, is an increase in proportion of cells achieving optimum plasticity.

In this set of experiments for producing cells having the ability to differentiate into different cell types, fresh human dermal fibroblasts derived from adult skin or from foreskin fibroblasts are cultured and detached by incubation with trypsin essentially as described in any one of examples 22 to 27.

Test cells are then recovered from culture immediately after trypsinization and are diluted to about 100,000 cells in 100 μl in high density plating medium (e.g., Medium 199 containing 170 nM insulin, 0.5 mM 3-isobutyl-1-methylxanthine, 0.2 mM indomethacin, 1 μM dexamethasone, and 15% rabbit serum). Within about 4 to 6 hours after trypsinization, test cells are recovered from culture and seeded at concentrations of about 100,000 cells per well/plate or at about 3703.7 cells per mm² surface area of the well/plate before attachment of the cells to the plate/well directly in 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum) for a time and under conditions sufficient for an optimum number of progenitor cells to be produced e.g., for up to about 24 hours or until adherence is achieved i.e., a shorter time than required for cells to become adherent and/or as determined by analysis of cell marker expression and/or by the ability of aliquots of cells to subsequently undergo differentiation.

As a negative control for the production of progenitor cells, trypsinized cells are seeded at a reduced density i.e., about 740.1 cells per mm² surface area, in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and incubated as for samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation.

Differentiation into Adipocytes

For differentiation into adipocytes, cells are incubated in adipogenic medium (Medium 199 containing 170 nM insulin, 0.5 mM 3-isobutyl-1-methylxanthine, 0.2 mM indomethacin, 1 dexamethasone, and 15% rabbit serum) at high density and allowed to expand for about 10-21 days.

As a negative control for differentiation, trypsinized cells are seeded at high density in high density plating medium (e.g., DMEM-HG supplemented with 10% FBS) and incubated as for test samples seeded at high density e.g., for up to about 24 hours or until adherent and/or as determined by analysis of cell marker expression and/or by the ability of aliquots of cells to subsequently undergo differentiation. The high density plating medium is then replaced with 200 to 400 μl DMEM-HG (10% FCS) medium and cells are allowed to expand for about 10-21 days.

As positive control for differentiation, rat bone marrow stromal/stem cells (rBMSCs) are expanded in DMEM medium containing L-Glutamine and 10% FBS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 400 μl DMEM-HG containing 10% FBS for up to about 24 hours or until adherent. The medium is replaced from adherent culture with 200 to 400 μl adipogenic medium (Medium 199 containing 170 nM insulin, 0.5 mM 3-isobutyl-1-methylxanthine, 0.2 mM indomethacin, 1 μM dexamethasone, and 15% rabbit serum) and cells are allowed to expand for about 10-21 days. Medium is replaced every 3 days for both test cells and negative and positive control cells.

### Assessment of Adipogenesis

After incubation for 12-21 days in adipogenic medium, differentiation potential of test cells compared to control cells at each day of incubation at high density post incubation optionally with low-serum and trypsinization is measured by an assessment of adipogenesis as described in example 2.

### Example 29

### Preparation of Cells Having the Ability to Differentiate into Other Cell Types by Induction of the Akt/(PKB) Pathway: Method 1

The data in Examples 22 to 28 suggest to the inventor that agonism of the Akt/(PKB) pathway and/or NF-κB pathway may produce equivalent or improved results as the combined action incubation in the presence of a modulator of AMPK and incubation in the presence of a protease such as trypsin to detach cells. Without being bound by any theory or mode of action, the inventor reasoned that modulation of AMPK and detachment of the cells to induce optimum plasticity of fibroblasts coincided with the induction of the Akt/(PKB) pathway, and that the responses of cells to the combined modulation of AMPK and trypsinization conditions is likely to induce the Akt/(PKB) pathway. Accordingly, the inventor sought to test whether or not modulation of AMPK and incubation in the presence of a protease such as trypsin could be reproduced or improved upon by incubation in the presence of one or more agonists of the Akt/(PKB) pathway. A possible advantage of using an agonist to induce the Akt/(PKB) pathway, as opposed to incubating cells with a modulator of 5′AMP-activated protein kinase or AMPK followed by trypsinization, or in concert with such a process, is enhancement of proportion of cells achieving optimum plasticity. By enhancing induction of the Akt/(PKB) pathway using an agonist compound, differentiated primary cells and cell lines that would normally enter a quiescent state or undergo apoptosis following modulation of AMPK can be used to produce cells capable of differentiating into different cell types.

In one example to show that Akt/(PKB) pathway induction confers or enhances plasticity of fibroblasts, primary human foreskin fibroblasts are incubated in the presence of human recombinant PDGF-BB for a time and under conditions sufficient to induce the Akt/(PKB) athway.

Production of Cells Capable of Differentiating into Different Cell Types

Fresh human dermal fibroblasts that are derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA (low-protein) for 24 hours to precondition the cells for PDGF-BB. After 24 hours, the medium is replaced with low-serum or serum-free DMEM containing 10 to 100 ng/ml of human recombinant PDGF-BB (Invitrogen) for 5 to 15 min to activate the Akt/(PKB) pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (e.g., Lonza) (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 30

### Preparation of Cells Having the Ability to Differentiate into Other Cell Types by Induction of the Akt/(PKB) Pathway: Method 2

In a further example to show that Akt/(PKB) pathway induction confers or enhances plasticity of fibroblasts, primary fibroblasts are incubated in the presence of TGF-β for a time and under conditions sufficient to induce the Akt/(PKB) pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA) (low-protein) for 24 hours to precondition the cells for TGF-β. After 24 hours, the medium is replaced with serum-free or low-serum DMEM-HG (e.g., Lonza) containing 1 to 10 ng/ml of TGF-β (R&D systems) for at least 60 min to activate the Akt/(PKB) pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (e.g., Lonza) (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 31

### Preparation of Cells Having the Ability to Differentiate into Other Cell Types by Induction of the Akt/(PKB) Pathway: Method 3

In a further example to show that Akt/(PKB) pathway induction confers or enhances plasticity of fibroblasts, primary fibroblasts are incubated in the presence of sodium pyruvate for a time and under conditions sufficient to induce the Akt/(PKB) pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or bovine serum albumin (BSA) (low-protein) for 24 hours to precondition the cells for sodium pyruvate. After 24 hours, the medium is replaced with serum-free or low-serum DMEM-HG containing 50 to 200 mg/L of cell culture grade sodium pyruvate (e.g., Lonza), and preferably, at 110 mg/L for at least 1 h to activate the Akt/(PKB) pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG with sodium pyruvate (e.g., Lonza Cat #12-604) (10% FBS) and maintained in serum-free medium until required for re-differentiation.

Re-differentiation of the cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 32

### Preparation of Cells Having the Ability to Differentiate into Other Cell Types by Induction of the Akt/(PKB) Pathway: Method 4

In a further example to show that Akt/(PKB) pathway induction confers or enhances plasticity of fibroblasts, mouse dermal primary fibroblasts are incubated in the presence of PDGF-BB for a time and under conditions sufficient to induce the Akt/(PKB) pathway.

Production of Cells Capable of Differentiating into Different Cell Types

Mouse dermal fibroblast cells are prepared from 8-12 week-old C57BL/6 mice. Briefly, mice are anesthetized with pentobarbital (50 mg/kg body weight), and a full thickness of the back skin is cut out by scissors. The skin tissues are cut into small pieces and are implanted into plastic tissue culture dishes containing DMEM-HG (e.g., Lonza) with 10% FBS. The fibroblast cultures are used after three to seven passages.

Adherent fibroblast cultures are incubated in DMEM-HG supplemented with 0-1% FBS or bovine serum albumin (BSA) (low-protein) for 48 hours to precondition the cells for PDGF-BB. After 48 hours, the medium is replaced with serum-free DMEM-HG or low-serum DMEM-HG containing 10 to 100 ng/ml of human recombinant PDGF-BB (Invitrogen) for 15 to 60 min to activate the Akt/(PKB)/(PKB) pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the treated cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 33

### Preparation of Cells Having the Ability to Differentiate into Other Cell Types by Induction of the Akt/(PKB) Pathway: Method 5

In a further example to show that Akt/(PKB) pathway induction confers or enhances plasticity of cells generally, rat adrenal cells are incubated in the presence of Carbachol or NGF for a time and under conditions sufficient to induce the Akt/(PKB) pathway.

Production of Cells Capable of Differentiating into Different Cell Types

PC12 cells are obtained from the American Type Culture Collection (CRL-1721, Rockville, Md.). PC12 cells are cultured in DMEM-HG supplemented with 5% (v/v) fetal calf serum and 10% (v/v) heat-inactivated horse serum, and grown at 37° C. in an environment of 7.5% CO₂ as described previously (Yu et al, Neurosignals 13: p248 (2004).

Adherent PC12 cultures are incubated in DMEM supplemented with 0-1% FBS or bovine serum albumin (BSA) (low-protein) for 24 hours to precondition the cells for Carbachol or NGF. After 24 hours, the medium is replaced with serum-free DMEM-HG or low-serum DMEM containing 200-1000 μM Carbachol (Calbiochem) or at least 50 ng/ml purified NGF (2.5S) (Alomone Labs Ltd) for 5 to 10 min to activate the Akt/(PKB) pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 34

### Preparation of Cells Having the Ability to Differentiate into Other Cell Types by Induction of the Akt/(PKB) Pathway: Method 6

In a further example to show that Akt/(PKB) pathway induction confers or enhances plasticity of cells generally, embryo fibroblasts are incubated in the presence of insulin growth factor-1 (IGF-1) for a time and under conditions sufficient to induce the Akt/(PKB) pathway.

Production of Cells Capable of Differentiating into Different Cell Types

Non-transformed rat embryo fibroblasts (Rat-1) are prepared and maintained as previously described (Peterson, et al., J. Biol. Chem. 271:31562-31571 (1996)).

Adherent Rat-1 cultures are incubated in DMEM-HG supplemented with 0-1% FBS or bovine serum albumin (low-protein) for 12 hours to precondition the cell for IGF-1. After 12 hours, the medium is replaced with serum-free DMEM or low-serum DMEM containing at least 250 ng/ml of insulin growth factor-1 (IGF-1; Sigma) for at least about 20 min to activate the Akt/(PKB) pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Differentiation into Other Cell Types

Re-differentiation of the treated cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 35

### Preparation of Cells Having the Ability to Differentiate into Other Cell Types by Induction of the NF-κB Pathway: Method 1

The data in examples 1 to 7 also suggest to the inventor that agonism of the NF-κB pathway may produce equivalent or improved results as the combined action of incubation in the presence of a modulator of 5′AMP-activated protein kinase or AMPK and incubation in the presence of a protease such as trypsin to detach cells. Without being bound by any theory or mode of action, the inventor reasoned that modulation of AMPK and detachment of the cells to induce optimum plasticity of fibroblasts coincided with induction of the NF-κB pathway, and that the responses of cells to the combined of AMPK and trypsinization conditions is likely to induce the NF-κB pathway. Accordingly, the inventor sought to test whether or not the effect of modulation of AMPK and incubation in the presence of a protease such as trypsin could be reproduced or improved upon by incubation in the presence of one or more agonists of the NF-κB pathway. An advantage of using an agonist to induce the NF-κB pathway, in concert with or as opposed to with a modulator of 5′AMP-activated protein kinase or AMPK followed by trypsinization, is enhancing the proportion of cells achieving optimum plasticity. By enhancing induction of the NF-κB pathway using an agonist, differentiated primary cells and cell lines that would normally enter a quiescent state or undergo apoptosis following modulation of AMPK can be used to produce cells capable of differentiating into different cell types.

In one example to show that NF-κB pathway induction confers or enhances plasticity of cells generally, primary human dermal fibroblasts are incubated in the presence of TNF-α for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with serum-free DMEM or low-serum DMEM-HG containing at least 20 ng/ml of TNF-α (Roche) for at least 60 min to activate the NF-κB pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 36

### Preparation of Cells Having the Ability to Differentiate into Other Cell Types by Induction of the NF-κB Pathway: Method 2

In a further example to show that NF-κB pathway induction confers or enhances plasticity of cells generally, primary human dermal fibroblasts are incubated in the presence of interleukin-1α for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with DMEM supplemented with 0.25% FBS of BSA for 50 hours to precondition the cells to interleukin-1α. After 50 hours, the cells are treated with recombinant human IL-1α at a concentration of least 0.27 ng/ml to activate the NF-κB pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Differentiation into Other Cell Types

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 37

### Preparation of Cells Having the Ability to Differentiate into Other Cell Types by Induction of the NF-κB Pathway: Method 3

In a further example to show that NF-κB pathway induction confers or enhances plasticity of fibroblasts, primary fibroblasts are incubated in the presence of sodium pyruvate for a time and under conditions sufficient to induce the NF-κB pathway.

Production of Cells Capable of Differentiating into Different Cell Types

Fresh human dermal fibroblasts derived from adult skin or from foreskin are are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or BSA (low-protein) for 24 hours to precondition the cells to sodium pyruvate treatment. After 24 hours, the medium is replaced with serum-free or low-serum DMEM-HG with sodium pyruvate containing 50 to 200 mg/L of cell culture grade sodium pyruvate (e.g., Lonza), and preferably, at 110 mg/L for at least 1 h to activate the NF-κB pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG with sodium pyruvate (e.g., Lonza Cat #12-604) (10% FBS) and maintained in this medium until required for re-differentiation.

Differentiation into Other Cell Types

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 38

### Preparation of Cells Having the Ability to Differentiate into Other Cell Types by Induction of the NF-κB Pathway: Method 4

In a further example to show that NF-κB pathway induction confers or enhances plasticity of cells generally, mouse embryo fibroblasts are incubated in the presence of L-alpha-Lysophosphatidic acid (C18:1, [cis]-9), LPA for a time and under conditions sufficient to induce the NF-κB pathway.

Production of Cells Capable of Differentiating into Different Cell Types

Swiss 3T3 mouse embryo fibroblasts are obtained from the American Type Culture Collection (CCL-92, Rockville, Md.) and are cultured at 37 C under a humidified atmosphere of 10% CO₂ in Dulbecco's modified Eagle's medium (DMEM) containing 10% (v/v) fetal calf serum.

Adherent 3T3 fibroblast cultures are incubated in DMEM-HG supplemented with 1% FBS or BSA (low-protein) for 18 hours to precondition the cells to L-alpha-Lysophosphatidic acid (C18:1, [cis]-9), LPA treatment. After 18 hours, L-α-Lysophosphatidic acid (C18:1,[cis]-9), LPA (Calbiochem; prepared as a stock of 1 mg/ml in phosphate-buffered saline containing 10 mg/ml essentially fatty acid-free bovine serum albumin (Sigma) is added to adherent cultures at 40-100 μM final concentration for about 40-120 min to activate the NF-κB pathway. As a control, TNF-α (Roche) is added to separate parallel cultures at a final concentration of 30 ng/ml for the same time period to activate the NF-κB pathway e.g., as described in Example 13.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 mM or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Differentiation into Other Cell Types

Re-differentiation of the adrenal into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 81 to 84.

### Example 39

### Preparation of Cells Having the Ability to Differentiate into Other Cell Types by Induction of the NF-κB Pathway: Method 5

In a further example to show that NF-κB pathway induction confers or enhances plasticity of cells generally, human myometrial microvascular endothelial cells (HUMEC) are incubated in the presence of Lipopolysaccharide (LPS) for a time and under conditions sufficient to induce the NF-κB pathway.

Production of Cells Capable of Differentiating into Different Cell Types

Human myometrial microvascular endothelial cells (HUMEC) are obtained from Technoclone GmbH (Vienna, Austria) and are cultured at 37 C in endothelial growth medium according to the specifications supplied by Technoclone GmbH.

Adherent HUMEC cultures are then incubated in endothelial medium, preferably serum free or containing low-serum concentration, and supplemented with 10-100 ng/ml of Lipopolysaccharide (LPS; Sigma) for at least 45 min to activate the NF-κB pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or 10⁻⁷ M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cells types are known in the art and described herein e.g., Examples 1, 2 and 81 to 84.

### Example 40

### Preparation of Cells Having the Ability to Differentiate into Other Cell Types by Induction of the NF-κB Pathway: Method 6

In a further example to show that NF-κB pathway induction confers or enhances plasticity of cells generally, synovial fibroblasts are incubated in the presence of Lipopolysaccharide (LPS) for a time and under conditions sufficient to induce the NF-κB pathway.

Production of Cells Capable of Differentiating into Different Cell Types

Primary cultures of synovial fibroblasts are obtained and maintained in culture as described previously (Brinckerhoff, and Mitchell, Journal of Cellular Physiology, 136 (1):72-80 (2005)). Adherent synovial fibroblast cultures are then incubated in growth medium, preferably serum free or containing low-serum concentration, and supplemented with 10-100 ng/ml of Lipopolysaccharide (LPS; Sigma) for at least 45 min to activate the NF-κB pathway.

The medium is then replaced with DMEM-LG containing 0-3 mM glucose supplemented about 0.5 mM to about 1.0 mM AICAR for 24 hours. Alternatively, the medium is replaced with DMEM-HG supplemented with 2 mM AICAR for 60 minutes, and incubated as described in Example 22. Control cells are incubated with the same medium as test cells without AICAR. Alternatively, the medium is replaced with DMEM-LG containing 0-3 mM glucose supplemented with 2 mM Metformin for 18 hours, and incubated as described in example 23. Control cells are incubated with the same medium as test cells without Metformin. Alternatively, the medium is replaced with DMEM-LG containing 0-3 mM glucose supplemented with 10 μM Compound C for 18 hours or 20 mM of Compound C for 60 minutes, and incubated as described in Example 24. Control cells are incubated with the same medium as test cells without Compound C. Alternatively, the medium is replaced with DMEM-HG containing supplemented with 2 U/ml Thrombin for 15 min, and incubated as described in Example 25. Control cells are incubated with the same medium as test cells without Thrombin. Alternatively, the medium is replaced with DMEM-HG supplemented with 10⁻⁶ M Ghrelin for 60 min or M Ghrelin for 90 min or with 10⁻⁹ M Ghrelin for 6 hours, and incubated as described in Example 26. Control cells are incubated with the same medium as test cells without Ghrelin.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cells types are known in the art and described herein e.g., Examples 1, 2 and 81 to 84.

### Example 41

### Preparation of Cells Having the Ability to Differentiate into Other Cell Types by Incubation in Medium Containing a Phorbol Ester or Active Derivative Thereof with or without Treatment with Protease: Method 1

In a this set of experiments for producing cells having the ability to differentiate into different cell types, fibroblasts are cultured and then either incubated in media with or without PMA [4β-12-O-tetradecanoylphorbol-13-acetate] and then are either incubated in media without trypsin or containing trypsin. The cells produced by this method are then tested for their ability to differentiate into adipocytes, as determined by the accumulation of fat.

### Preparation of PMA

Stock solution of PMA (Sigma Chemical Co., St Louis, Mo., USA) are prepared by dissolving in ethanol or dimethyl sulfoxide (DMSO), such that the final diluent concentration of DMSO s 0.1% (v/v) in all experiments. Stock solutions are stored at −20° C., until required.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) supplemented with about 100 to 200 nM final concentration of PMA (Sigma) dissolved ethanol or DMSO for about 3 hr to 16 hrs or about 1 μM final concentration of PMA (Sigma) dissolved in ethanol or DMSO for about 10 min to 1 hr. Control cells are incubated with the same medium as test cells without PMA but in the same final diluent concentration of carrier i.e., ethanol or DMSO.

At the conclusion of the incubation period in media containing PMA, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), cells are then detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Alternatively, at the conclusion of the incubation period in media containing PMA, cells are washed in PBS and the medium is replaced with DMEM-HG supplemented with 10% FBS, without protease, and incubated at 37° C. without detaching the cells. Cells are maintained in this medium until required for re-differentiation. Control cells are either detached or not detached as above, and are used directly in the differentiation assay as described below. Differentiation into adipocytes and assessment of adipogenesis is carried out as described in Example 2.

For example, differentiation of the cell product into other cell types is achieved by reseeding the cells described above into differentiation media for adipocytes, or as described herein e.g., Examples 1, 2 and 81 to 84.

### Example 42

### Preparation of Cells Having the Ability to Differentiate into Other Cell Types by Incubation in Medium Containing a Phorbol Ester or Active Derivative Thereof and Treatment with Protease: Method 2

In a this set of experiments for producing cells having the ability to differentiate into different cell types, fibroblasts are cultured and then either incubated in media with or without PDBu [4β-phorbol-12,13-dibutyrate] and then are either incubated in media without trypsin or containing trypsin. The cells produced by this method are then tested for their ability to differentiate into adipocytes, as determined by the accumulation of fat.

### Preparation of PDBu

Stock solution of PDBu (Sigma Chemical Co., St Louis, Mo., USA) are prepared by dissolving in ethanol or dimethyl sulfoxide (DMSO), such that the final diluent concentration of DMSO 0.1% (v/v) in all experiments. Stock solutions are stored at −20° C., until required.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) supplemented with about 100 to 200 nM final concentration of PDBu (Sigma) dissolved ethanol or DMSO for 48 to 72 hrs or 1 μM final concentration of PDBu (Sigma) dissolved in ethanol or DMSO for about 30 min to about 1 hr. Control cells are incubated with the same medium as test cells without PDBu but in the same final diluent concentration of carrier i.e., ethanol or DMSO.

At the conclusion of the incubation period in media containing PDBu, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Alternatively, at the conclusion of the incubation period in media containing PDBu, cells are washed in PBS and the medium is replaced with DMEM-HG supplemented with 10% FBS, without protease, and incubated at 37° C. without detaching the cells. Cells are maintained in this medium until required for re-differentiation. Control cells are either detached or not detached as described above, and are used directly in the differentiation assay as described in Example 2. Differentiation into adipocytes and assessment of adipogenesis is carried out as described in Example 2.

For example, differentiation of the cell product into other cell types is achieved by reseeding the cells described in this example into differentiation media for adipocytes, or as described herein e.g., Examples 1, 2 and 81 to 84.

### Example 43

### Preparation of Cells Having the Abilit to Differentiate into Other Cell Types by Incubation in Medium Containing a Phorbol Ester or Active Derivative Thereof and with or without Treatment with protease: Method 3

In a this set of experiments for producing cells having the ability to differentiate into different cell types, fibroblasts are cultured and then either incubated in media with or without phorbol sapintoxin D or in a medium with or without phorbol sapintoxin A and then are either incubated in media without trypsin or containing trypsin. The cells produced by this method are then tested for their ability to differentiate into adipocytes, as determined by the accumulation of fat.

### Preparation of Phorbol Sapintoxin A or Phorbol Sapintoxin D

Stock solution of phorbol sapintoxin A or phorbol sapintoxin D (e.g., purchased from Calbiochem, San Diego, Calif., USA or LC Laboratories, Woburn, Mass. USA) are prepared by dissolving in ethanol or dimethyl sulfoxide (DMSO), such that the final diluent concentration of DMSO 0.1% (v/v) in all experiments. Stock solutions are stored at −20° C., until required.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) supplemented with about 100 to 200 nM final concentration of sapintoxin A (Calbiochem) or sapintoxin D (LC Laboratories) dissolved ethanol or DMSO for about 7 hr to about 24 hrs. Control cells are incubated with the same medium as test cells without phorbol sapintoxin A or D but in the same final diluent concentration of carrier i.e., ethanol or DMSO.

At the conclusion of the incubation period in media containing phorbol sapintoxin A or phorbol sapintoxin D, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 p. 1 with DMEM-HG (e.g., Lonza, Cat #12-604) (with 10% FBS) and maintained in this medium until required for re-differentiation. Alternatively, at the conclusion of the incubation period in media containing phorbol sapintoxin A or phorbol sapintoxin D, cells are washed in PBS and the medium is replaced with DMEM-HG supplemented with 10% FBS, without protease, and incubated at 37° C. without detaching the cells. Cells are maintained in this medium until required for re-differentiation. Control cells are either detached or not detached as described above, and are used directly in the differentiation assay as described in Example 2. Differentiation into Adipocytes and assessment of adipogenesis is carried out as described in Example 2.

For example, differentiation of the cell product into other cell types is achieved by reseeding the cells described in Example 1 into differentiation media for adipocytes, or as described herein e.g., Examples 1, 2 and 81 to 84.

### Example 44

### Preparation of Cells Having the Ability to Differentiate into Other Cell Types by Incubation in Medium Containing a Phorbol Ester or Active Derivative Thereof with or without Treatment with Protease, and with Additional Incubation in Low-Serum

The inventor sought to test whether or not the additional step of incubating cells in a low serum media may produce equivalent or improved results as the combined action of incubation with a phorbol ester or active derivative thereof and incubation with a protease or alternatively by agonism of the Akt/(PKB) and/or the NF-κB pathway using an agonist compound. Without being bound by any theory or mode of action, the inventors reasoned that low-serum incubation conditions for 5-9 days further induces/enhances activation of the Akt/(PKB) and/or the NF-κB pathway. A possible advantage of using low-serum incubation for 5-9 days in concert together with incubation with a phorbol ester or active derivative thereof and detachment of cells e.g., by incubation with a protease such as trypsin and/or inducing the Akt/(PKB) and/or the NF-κB pathway using an agonist compound, is an increase in proportion of cells achieving optimum plasticity and/or enhanced survival under incubation conditions with a phorbol ester or active derivative thereof.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per mm² surface area. Once all cells are attached, the medium is replaced with medium 199 (M199) (e.g., Sigma) supplemented with 0-1% FBS (low-serum) for different periods of time, from 1 to 11 days.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

At the conclusion of the incubation period in low serum media, test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with serum-free DMEM-HG (e.g., Lonza, Cat #12-604) (0% FBS) and maintained in serum-free medium until required for re-differentiation. Alternatively, at the conclusion of the incubation period in media containing a phorbol ester, cells are washed in PBS and the medium is replaced with DMEM-HG supplemented with 10% FBS, without protease, and incubated at 37° C. without detaching the cells. Cells are maintained in this medium until required for re-differentiation. Control cells are either detached or not detached as described above, and are used directly in the differentiation assay as described in Example 2. Differentiation into Adipocytes and assessment of adipogenesis is carried out as described in Example 2. The person skilled in the art would appreciate that differentiation of test cells into adipocytes may continue albeit at below optimum even after the 11-day period incubation at low serum.

For example, differentiation of the cell product into other cell types is achieved by reseeding the cells into differentiation media for adipocytes, or as described herein e.g., Examples 1, 2 and 81 to 84.

### Example 45

### Preparation of Cells Having the Ability to Differentiate into Other Cell Types by Incubation in Medium Containing a Phorbol Ester or Active Derivative Thereof and Treatment with Protease and with Additional Incubation at High Cell Density Conditions

To improve the yield of cells having the ability to differentiate into other cell types, the inventor sought to investigate the effect of high density cultures on plasticity. Specifically, the inventor sought to test whether or not the additional step of incubating cells at high density in a suitable differentiation media may produce equivalent or improved results as incubation with a phorbol ester or active derivative thereof and incubation in the presence of protease or alternatively by agonism of the Akt/(PKB) and/or the NF-κB pathway using an agonist compound. Without being bound by any theory or mode of action, the inventor reasoned that culturing protease treated cells at high cell density in a high density plating medium, further induces activation of the NF-κB pathway. A possible advantage of using a high cell density following protease treatment to induce the NF-κB pathway, in concert with incubating cells with a phorbol ester or active derivative thereof and in the presence of a protease and/or inducing the Akt/(PKB) and/or the NF-κB pathway using an agonist compound, is an increase in proportion of cells achieving optimum plasticity.

In this set of experiments for producing cells having the ability to differentiate into different cell types, fresh human dermal fibroblasts derived from adult skin or from foreskin fibroblasts are cultured and detached by incubation with trypsin essentially as described in any one of examples 41 to 44.

Test cells are then recovered from culture immediately after trypsinization and are diluted to about 100,000 cells in 100 μl in high density plating medium (e.g., Medium 199 containing 170 nM insulin, 0.5 mM 3-isobutyl-1-methylxanthine, 0.2 mM indomethacin, 1 μM dexamethasone, and 15% rabbit serum). Within about 4 to 6 hours after trypsinization, test cells are recovered from culture and seeded at concentrations of about 100,000 cells per well/plate or at about 3703.7 cells per mm² surface area of the well/plate before attachment of the cells to the plate/well directly in 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum) for a time and under conditions sufficient for an optimum number of progenitor cells to be produced e.g., for up to about 24 hours or until adherence is achieved i.e., a shorter time than required for cells to become adherent and/or as determined by analysis of cell marker expression and/or by the ability of aliquots of cells to subsequently undergo differentiation.

As a negative control for the production of progenitor cells, trypsinized cells are seeded at a reduced density i.e., about 740.1 cells per mm² surface area, in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and incubated as for samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation.

Differentiation into Adipocytes

For differentiation into adipocytes, cells are incubated in adipogenic medium (Medium 199 containing 170 nM insulin, 0.5 mM 3-isobutyl-1-methylxanthine, 0.2 mM indomethacin, 1 μM dexamethasone, and 15% rabbit serum) at high density and allowed to expand for about 10-21 days.

As a negative control for differentiation, trypsinized cells are seeded at high density in high density plating medium (e.g., DMEM-HG supplemented with 10% FBS) and incubated as for test samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation. The high density plating medium is then replaced with 200 to 400 μl DMEM-HG (10% FCS) medium and cells are allowed to expand for about 10-21 days.

As positive control for differentiation, rat bone marrow stromal/stem cells (rBMSCs) are expanded in DMEM medium containing L-Glutamine and 10% FCS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 400 μl DMEM-HG containing 10% FCS for up to about 24 hours or until adherent. The medium is replaced from adherent culture with 200 to 400 μl adipogenic medium (Medium 199 containing 170 nM insulin, 0.5 mM 3-isobutyl-1-methylxanthine, 0.2 mM indomethacin, 1 μM dexamethasone, and 15% rabbit serum) and cells are allowed to expand for about 10-21 days.

Medium is replaced every 3 days for both test cells and negative and positive control cells.

### Assessment of Adipogenesis

After incubation for 12-21 days in adipogenic medium, differentiation potential of test cells compared to control cells at each day of incubation at high density post incubation optionally with low-serum and trypsinization is measured by an assessment of adipogenesis as described in example 2.

### Example 46

### Preparation of Cells Having the Ability to Differentiate into Other Cell Types by Induction of the Akt/(PKB) Pathway: Method 1

The data in Examples 41 to 45 suggest to the inventor that agonism of the Akt/(PKB) pathway and/or NF-κB pathway may produce equivalent or improved results as the combined action incubation in the presence of a phorbol ester or active derivative thereof and incubation in the presence of a protease such as trypsin to detach cells. Without being bound by any theory or mode of action, the inventor reasoned that incubation with a phorbol ester or active derivative thereof and detachment of the cells to induce optimum plasticity of fibroblasts coincided with the induction of the Akt/(PKB) pathway, and that the responses of cells to the combined phorbol ester and trypsinization conditions is likely to induce the Akt/(PKB) pathway. Accordingly, the inventor sought to test whether or not incubation with a phorbol ester or active derivative thereof and incubation in the presence of a protease such as trypsin could be reproduced or improved upon by incubation in the presence of one or more agonists of the Akt/(PKB) pathway. A possible advantage of using an agonist to induce the Akt/(PKB) pathway, as opposed to incubating cells a phorbol ester or active derivative thereof followed by trypsinization, or in concert with such a process, is enhancement of cell survival and the proportion of cells achieving optimum plasticity. By enhancing induction of the Akt/(PKB) pathway using an agonist compound, differentiated primary cells and cell lines that would normally enter a quiescent state or undergo apoptosis as a result of exposure to a phorbol ester or active derivative thereof can be used to produce cells capable of differentiating into different cell types.

In one example to show that Akt/(PKB) pathway induction confers or enhances plasticity of fibroblasts, primary human foreskin fibroblasts are incubated in the presence of human recombinant PDGF-BB for a time and under conditions sufficient to induce the Akt/(PKB) pathway.

Production of Cells Capable of Differentiating into Different Cell Types

Fresh human dermal fibroblasts that are derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA (low-protein) for 24 hours to precondition the cells for PDGF-BB. After 24 hours, the medium is replaced with low-serum or serum-free DMEM containing 10 to 100 ng/ml of human recombinant PDGF-BB (Invitrogen) for 5 to 15 min to activate the Akt/(PKB) pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

In one example, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (e.g., Lonza) (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 47

### Preparation of Cells Having the Ability to Differentiate into Other Cell Types by Induction of the Akt/(PKB) Pathway: Method 2

In a further example to show that Akt/(PKB) pathway induction confers or enhances plasticity of fibroblasts, primary fibroblasts are incubated in the presence of TGF-(3 for a time and under conditions sufficient to induce the Akt/(PKB) pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA) (low-protein) for 24 hours to precondition the cells for TGF-β. After 24 hours, the medium is replaced with serum-free or low-serum DMEM-HG (e.g., Lonza) containing 1 to 10 ng/ml of TGF-β (R&D systems) for at least 60 min to activate the Akt/(PKB) pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, then diluted to 200 μl with DMEM-HG (e.g., Lonza) (10% FBS) and maintained in this medium until required for re-differentiation. For example, differentiation of the cell product into other cell types is achieved by reseeding the cells described above into differentiation media for adipocytes, or as described herein e.g., Examples 18 to 21.

### Example 48

### Preparation of Cells Having the Ability to Differentiate into Other Cell Types by Induction of the Akt/(PKB) Pathway: Method 3

In a further example to show that Akt/(PKB) pathway induction confers or enhances plasticity of fibroblasts, primary fibroblasts are incubated in the presence of sodium pyruvate for a time and under conditions sufficient to induce the Akt/(PKB) pathway.

Production of Cells Capable of Differentiating into Different Cell Types

Fresh human dermal fibroblasts derived from adult skin or from foreskin are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or bovine serum albumin (BSA) (low-protein) for 24 hours to precondition the cells for sodium pyruvate. After 24 hours, the medium is replaced with serum-free or low-serum DMEM-HG containing 50 to 200 mg/L of cell culture grade sodium pyruvate (e.g., Lonza), and preferably, at 110 mg/L for at least 1 h to activate the Akt/(PKB) pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG with sodium pyruvate (e.g., Lonza Cat #12-604) (10% FBS) and maintained in serum-free medium until required for re-differentiation.

For example, differentiation of the cell product into other cell types is achieved by reseeding the cells described above into differentiation media for adipocytes, or as described herein e.g., Examples 1, 2 and 81 to 84.

### Example 49

### Preparation of Cells Having the Ability to Differentiate into Other Cell Types by Induction of the Akt/(PKB) Pathway: Method 4

In a further example to show that Akt/(PKB) pathway induction confers or enhances plasticity of fibroblasts, mouse dermal primary fibroblasts are incubated in the presence of PDGF-BB for a time and under conditions sufficient to induce the Akt/(PKB) pathway.

Production of Cells Capable of Differentiating into Different Cell Types

Mouse dermal fibroblast cells are prepared from 8-12 week-old C57BL/6 mice. Briefly, mice are anesthetized with pentobarbital (50 mg/kg body weight), and a full thickness of the back skin is cut out by scissors. The skin tissues are cut into small pieces and are implanted into plastic tissue culture dishes containing DMEM-HG (e.g., Lonza) with 10% FBS. The fibroblast cultures are used after three to seven passages.

Adherent fibroblast cultures are incubated in DMEM-HG supplemented with 0-1% FBS or bovine serum albumin (BSA) (low-protein) for 48 hours to precondition the cells for PDGF-BB. After 48 hours, the medium is replaced with serum-free DMEM-HG or low-serum DMEM-HG containing 10 to 100 ng/ml of human recombinant PDGF-BB (Invitrogen) for 15 to 60 min to activate the Akt/(PKB)/(PKB) pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

For example, differentiation of the cell product into other cell types is achieved by reseeding the cells described above into differentiation media for adipocytes, or as described herein e.g., Examples 1, 2 and 81 to 84.

### Example 50

### Preparation of Cells Having the Ability to Differentiate into Other Cell Types by Induction of the Akt/(PKB) Pathway: Method 5

In a further example to show that Akt/(PKB) pathway induction confers or enhances plasticity of cells generally, rat adrenal cells are incubated in the presence of Carbachol or NGF for a time and under conditions sufficient to induce the Akt/(PKB) pathway.

Production of Cells Capable of Differentiating into Different Cell Types

PC12 cells are obtained from the American Type Culture Collection (CRL-1721, Rockville, Md.). PC12 cells are cultured in DMEM-HG supplemented with 5% (v/v) fetal calf serum and 10% (v/v) heat-inactivated horse serum, and grown at 37° C. in an environment of 7.5% CO₂ as described previously (Yu et al, Neurosignals 13: p248 (2004) incorporated herein by reference in its entirety.

Adherent PC12 cultures are incubated in DMEM supplemented with 0-1% FBS or bovine serum albumin (BSA) (low-protein) for 24 hours to precondition the cells for Carbachol or NGF. After 24 hours, the medium is replaced with serum-free DMEM-HG or low-serum DMEM containing 200-1000 μM Carbachol (Calbiochem) or at least 50 ng/ml purified NGF (2.5S) (Alomone Labs Ltd) for 5 to 10 min to activate the Akt/(PKB) pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

For example, differentiation of the cell product into other cell types is achieved by reseeding the cells described above into differentiation media for adipocytes, or as described herein e.g., Examples 1, 2 and 81 to 84.

### Example 51

### Preparation of Cells Having the Ability to Differentiate into Other Cell Types by Induction of the Akt/(PKB) Pathway: Method 6

In a further example to show that Akt/(PKB) pathway induction confers or enhances plasticity of cells generally, embryo fibroblasts are incubated in the presence of insulin growth factor-1 (IGF-1) for a time and under conditions sufficient to induce the Akt/(PKB) pathway.

Production of Cells Capable of Differentiating into Different Cell Types

Non-transformed rat embryo fibroblasts (Rat-1) are prepared and maintained as previously described (Peterson, et al., J. Biol. Chem. 271:31562-31571 (1996)).

Adherent Rat-1 cultures are incubated in DMEM-HG supplemented with 0-1% FBS or bovine serum albumin (low-protein) for 12 hours to precondition the cell for IGF-1. After 12 hours, the medium is replaced with serum-free DMEM or low-serum DMEM containing at least 250 ng/ml of insulin growth factor-1 (IGF-1; Sigma) for at least about 20 min to activate the Akt/(PKB) pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

For example, differentiation of the cell product into other cell types is achieved by reseeding the cells described above into differentiation media for adipocytes, or as described herein e.g., Examples 1, 2 and 81 to 84.

### Example 52

### Preparation of Cells Having the Ability to Differentiate into Other Cell Types by Induction of the NF-κB Pathway: Method 1

The data in examples 41 to 45 also suggest to the inventor that agonism of the NF-κB pathway may produce equivalent or improved results as the combined action of incubation in the presence of a phorbol ester or active derivative thereof and incubation in the presence of a protease such as trypsin to detach cells. Without being bound by any theory or mode of action, the inventor reasoned that incubation in the presence of a phorbol ester or active derivative thereof and detachment of the cells to induce optimum plasticity of fibroblasts coincided with induction of the NF-κB pathway, and that the responses of cells to the combined phorbol ester and trypsinization conditions is likely to induce the NF-κB pathway. Accordingly, the inventor sought to test whether or not the effect of incubation in the presence of a phorbol ester or active derivative thereof and incubation in the presence of a protease such as trypsin could be reproduced or improved upon by incubation in the presence of one or more agonists of the NF-κB pathway. A possible advantage of using an agonist to induce the NF-κB pathway, in concert with or as opposed to incubating cells with a phorbol ester or active derivative thereof followed by trypsinization, is enhancing the proportion of cells achieving optimum plasticity. By enhancing induction of the NF-κB pathway using an agonist, differentiated primary cells and cell lines that would normally enter a quiescent state or undergo apoptosis as a result of exposure to phorbol ester or active derivative thereof can be used to produce cells capable of differentiating into different cell types.

In one example to show that NF-κB pathway induction confers or enhances plasticity of cells generally, primary human dermal fibroblasts are incubated in the presence of TNF-α for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with serum-free DMEM or low-serum DMEM-HG containing at least 20 ng/ml of TNF-α (Roche) for at least 60 min to activate the NF-κB pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 53

### Preparation of Cells Having the Ability to Differentiate into Other Cell Types by Induction of the NF-κB Pathway: Method 2

In a further example to show that NF-κB pathway induction confers or enhances plasticity of cells generally, primary human dermal fibroblasts are incubated in the presence of interleukin-1α for a time and under conditions sufficient to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with DMEM supplemented with 0.25% FBS of BSA for 50 hours to precondition the cells to interleukin-1α. After 50 hours, the cells are treated with recombinant human IL-1α at a concentration of least 0.27 ng/ml to activate the NF-κB pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 54

### Preparation of Cells Having the Ability to Differentiate into Other Cell Types by Induction of the NF-κB Pathway: Method 3

In a further example to show that NF-κB pathway induction confers or enhances plasticity of fibroblasts, primary fibroblasts are incubated in the presence of sodium pyruvate for a time and under conditions sufficient to induce the NF-κB pathway.

Production of Cells Capable of Differentiating into Different Cell Types

Fresh human dermal fibroblasts derived from adult skin or from foreskin are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or BSA (low-protein) for 24 hours to precondition the cells to sodium pyruvate treatment. After 24 hours, the medium is replaced with serum-free or low-serum DMEM-HG with sodium pyruvate containing 50 to 200 mg/L of cell culture grade sodium pyruvate (e.g., Lonza), and preferably, at 110 mg/L for at least 1 h to activate the NF-κB pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG with sodium pyruvate (e.g., Lonza Cat #12-604) (10% FBS) and maintained in this medium until required for re-differentiation.

Re-differentiation of the treated fibroblasts into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 55

### Preparation of Cells Having the Ability to Differentiate into Other Cell Types by Induction of the NF-κB Pathway: Method 4

In a further example to show that NF-κB pathway induction confers or enhances plasticity of cells generally, mouse embryo fibroblasts are incubated in the presence of L-alpha-Lysophosphatidic acid (C18:1, [cis]-9), LPA for a time and under conditions sufficient to induce the NF-κB pathway.

Production of Cells Capable of Differentiating into Different Cell Types

Swiss 3T3 mouse embryo fibroblasts are obtained from the American Type Culture Collection (CCL-92, Rockville, Md.) and are cultured at 37 C under a humidified atmosphere of 10% CO₂ in Dulbecco's modified Eagle's medium (DMEM) containing 10% (v/v) fetal calf serum.

Adherent 3T3 fibroblast cultures are incubated in DMEM-HG supplemented with 1% FBS or BSA (low-protein) for 18 hours to precondition the cells to L-alpha-Lysophosphatidic acid (C18:1, [cis]-9), LPA treatment. After 18 hours, L-α-Lysophosphatidic acid (C18:1,[cis]-9), LPA (Calbiochem; prepared as a stock of 1 mg/ml in phosphate-buffered saline containing 10 mg/ml essentially fatty acid-free bovine serum albumin (Sigma) is added to adherent cultures at 40-100 μM final concentration for about 40-120 min to activate the NF-κB pathway. As a control, TNF-α (Roche) is added to separate parallel cultures at a final concentration of 30 ng/ml for the same time period to activate the NF-κB pathway e.g., as described in Example 53.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at room temperature until cells lifted from the plates. Treated cells are recovered from culture, then diluted to 200 μl with DMEM-HG (10% FBS) and maintained in this medium until required for re-differentiation.

Re-differentiation of the treated cell product into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1, 2 and 18 to 21.

### Example 56

### Preparation of Cells Having the Ability to Differentiate into Other Cell Types by Induction of the NF-κB Pathway: Method 5

In a further example to show that NF-κB pathway induction confers or enhances plasticity of cells generally, human myometrial microvascular endothelial cells (HUMEC) are incubated in the presence of Lipopolysaccharide (LPS) for a time and under conditions sufficient to induce the NF-κB pathway.

Production of Cells Capable of Differentiating into Different Cell Types

Human myometrial microvascular endothelial cells (HUMEC) are obtained from Technoclone GmbH (Vienna, Austria) and are cultured at 37 C in endothelial growth medium according to the specifications supplied by Technoclone GmbH.

Adherent HUMEC cultures are then incubated in endothelial medium, preferably serum free or containing low-serum concentration, and supplemented with 10-100 ng/ml of Lipopolysaccharide (LPS; Sigma) for at least 45 min to activate the NF-κB pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated 37° C. until cells lifted from the plates. Treated cells are recovered from culture, then diluted to 200 μl with DMEM-HG (10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 57

### Preparation of Cells Having the Ability to Differentiate into Other Cell Types by Induction of the NF-κB Pathway: Method 6

In a further example to show that NF-κB pathway induction confers or enhances plasticity of cells generally, synovial fibroblasts are incubated in the presence of Lipopolysaccharide (LPS) for a time and under conditions sufficient to induce the NF-κB pathway.

Production of Cells Capable of Differentiating into Different Cell Types

Primary cultures of synovial fibroblasts are obtained and maintained in culture as described previously (Brinckerhoff, and Mitchell, Journal of Cellular Physiology, 136 (1):72-80 (2005)).

Adherent synovial fibroblast cultures are then incubated in growth medium, preferably serum free or containing low-serum concentration, and supplemented with 10-100 ng/ml of Lipopolysaccharide (LPS; Sigma) for at least 45 min to activate the NF-κB pathway.

The medium is then replaced with DMEM-HG supplemented with about 100 to 200 nM PMA (Sigma) dissolved ethanol or in DMSO for about 3 hr to about 16 hrs or about 1 μM PMA (Sigma) dissolved in ethanol or DMSO for about 10 min to about 1 hr, as described in Example 41. Control cells are incubated with the same medium as test cells without PMA. Alternatively, the medium is replaced with DMEM-HG supplemented with about 100 to 200 nM of PDBu (Sigma) dissolved ethanol or in DMSO for about 48 to about 72 hrs or about 1 μM of PDBu (Sigma) dissolved in ethanol or DMSO for about 30 min to about 1 hr, as described in Example 42. Control cells are incubated with the same medium as test cells without PDBu. Alternatively, the medium is replaced with DMEM-HG supplemented 100 to 200 nM sapintoxin A (Calbiochem) or sapintoxin D (LC Laboratories) dissolved either in ethanol or DMSO for about 7 hr to about 24 hrs, as described in Example 43. Control cells are incubated with the same medium as test cells without phorbol sapintoxin A or D.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated 37° C. until cells lifted from the plates. Treated cells are recovered from culture, then diluted to 200 μl with DMEM-HG (10% FBS) and maintained in this medium until required for re-differentiation.

Re-differentiation of the treated fibroblasts cells into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 58

### Preparation of Cells Having the Ability to Differentiate into Other Cell Types by Incubation in Medium Comprising a Retinoid and Treatment with Protease: Method 1

In this set of experiments for producing cells having the ability to differentiate into different cell types, fibroblasts are cultured and then incubated in M199 medium (e.g. Sigman Cat #2154) which naturally had a constituent retinoic acid (retinol) at final concentration of about 10⁻⁸ M and then are either incubated in media without trypsin or containing trypsin. The cells produced by this method are then tested for their ability to differentiate into adipocytes.

Production of Cells Capable of Differentiating into Other Cell Types

Fresh human dermal fibroblasts derived from adult skin or from foreskin are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area.

Once all cells are attached, the medium is replaced with medium 199 (M199) (e.g., Sigma Cat #2154) containing retinoic acid as one of its constituents at final concentration of about 10⁻⁸ M and cells are incubated for about 7 days to about 9 days at 37° C. Control cells are incubated at 37° C. for the same period of time but in growth medium Dulbecco's Modified Eagle Medium High Glucose (DMEM-HG) (0% serum) which does not have retinoic acid, and without being supplemented with any retinoid. Media are changed every 48 hours for both the test and control cells.

At the conclusion of the incubation period, cells are washed in PBS and the medium is replaced with growth medium Dulbecco's Modified Eagle Medium High Glucose (DMEM-HG; e.g., Lonza Cat #12-604), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells described above into differentiation media for adipocytes, or as described herein e.g., Examples 1, 2 and 81 to 84.

### Example 59

### Preparation of Cells Having the Ability to Differentiate into Other Cell Types by Incubation in Medium Comprising a Retinoid and Treatment with Protease: Method 2

In this set of experiments for producing cells having the ability to differentiate into different cell types, fibroblasts are cultured and then incubated in a medium supplemented with serum. The inventor reasons that undiluted serum has a constituent retinoic acid (retinol) at final concentration of about 0.51×10⁻⁶ M (Ishida et al., Allergy. 58:1044-1052, which incorporated herein by reference in its entirety). The cells are then either incubated in media without trypsin or containing trypsin. The cells produced by this method are then tested for their ability to differentiate into adipocytes.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG), which does not comprise retinoic acid and is not supplemented with any retinoid, and the cells are incubated at 37° C. for about 7 days. Test cells are then washed with PBS and incubated in DMEM-HG supplemented with about 5% to about 50% FBS (fetal bovine serum) containing retinoic acid at final concentration of about 0.255×M to about 0.255×10⁻⁶M and incubated for a further about 5 to about 7 days at 37° C. Control cells are washed with PBS and incubated in DMEM-HG without any serum and without any retinoid for the same period of time as the test cells. Media are changed every 48 hrs for both control and test cells.

At the conclusion of the incubation period, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. No detachment control cells are not trypsinized, and are used directly in the differentiation assay as described in Example 2. Differentiation into adipocytes and assessment of adipogenesis is carried out as described in Example 2.

For example, differentiation of the cell product into other cell types is achieved by reseeding the cells described in this example into differentiation media for adipocytes, or as described herein e.g., Examples 1, 2 and 81 to 84.

### Example 60

### Preparation of Cells Having the Ability to Differentiate into Other Cell Types by Incubation in Medium Comprising a Retinoid and Treatment with Protease: Method 3

In this set of experiments for producing cells having the ability to differentiate into different cell types, fibroblasts are cultured and then either incubated in media with or without all-trans-retinoic acid (ATRA) and then are either incubated in media without trypsin or containing trypsin. The cells produced by this method are then tested for their ability to differentiate into adipocytes.

### Preparation of ATRA

Stock solution of ATRA (Sigma Chemical Co., St Louis, Mo., USA) are prepared by dissolving initially in dimethyl sulfoxide (DMSO) and then in ethanol, and finally in DMEM at a concentration of 10⁻⁵ M for use at a final concentration of 10⁻⁸ M to 10⁻⁶ M such that the final diluent concentration of ethanol is 0.1% (v/v) or less in all experiments. As ATRA is light sensitive, all experiments are performed in the dark and prepared fresh for each experiment.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10⁻⁸ M to 10⁻⁶M final concentration of ATRA (Sigma) dissolved ethanol for about 72 hr to 168 hrs, i.e., for about 3 to 7 days, with a change of medium every 48 hours. Control cells are incubated with the same medium as test cells without ATRA but in the same final diluent concentration of carrier i.e., ethanol.

At the conclusion of the incubation period in media containing ATRA, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells described in this example into differentiation media for adipocytes, or as described herein e.g., Examples 1, 2 and 81 to 84.

### Example 61

### Preparation of Cells Having the Ability to Differentiate into Other Cell Types by Incubation in Medium Comprising a Retinoid and Treatment with Protease: Method 4

In this set of experiments for producing cells having the ability to differentiate into different cell types, fibroblasts are cultured and then either incubated in media with or without 9-cis retinoic acid (9CRA) and then are either incubated in media without trypsin or containing trypsin. The cells produced by this method are then tested for their ability to differentiate into adipocytes.

### Preparation of 9CRA

Stock solution of 9CRA (Sigma Chemical Co., St Louis, Mo., USA) are prepared by dissolving initially in dimethyl sulfoxide (DMSO) and then in ethanol, and finally in DMEM at a concentration of 10⁻⁵ M for use at a final concentration of 10⁻⁸ M to 10⁻⁶ M such that the final diluent concentration of ethanol is 0.1% ON or less in all experiments. Stock solutions are prepared fresh for each experiment.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 0% to 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10⁻⁸M to 10⁻⁶ M final concentration of 9CRA (Sigma) dissolved ethanol for about 72 hr to 168 hrs, i.e., for about 3 to about 7 days, with a change of medium every 48 hours. Control cells are incubated with the same medium as test cells without 9CRA but in the same final diluent concentration of carrier i.e., ethanol.

At the conclusion of the incubation period in media containing 9CRA, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells into differentiation media for adipocytes, or as described herein e.g., Examples 1, 2 and 81 to 84.

### Example 62

### Preparation of Cells Having the Ability to Differentiate into Other Cell Types by Incubation in Medium Comprising a Retinoid and Treatment with Protease: Method 5

In this set of experiments for producing cells having the ability to differentiate into different cell types, fibroblasts are cultured and then either incubated in media with or without Am80 and then are either incubated in media without trypsin or containing trypsin. The cells produced by this method are then tested for their ability to differentiate into adipocytes, as determined by the accumulation of fat.

### Preparation of Am80

Stock solution of Am80 (e.g., Galderma Laboratories, Sophia, France) are prepared by dissolving in ethanol or dimethyl sulfoxide (DMSO), such that the final diluent concentration of DMSO 0.1% (v/v) in all experiments. Stock solutions are stored at −20° C., until required.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10⁻⁹ M to 10×10⁻⁶ M final concentration of Am80 (Galderma Laboratories) dissolved ethanol for about 72 hr to 168 hrs, i.e., for about 3 to about 7 days, with a change of medium every 48 hours. Control cells are incubated with the same medium as test cells without Am80 but in the same final diluent concentration of carrier i.e., ethanol.

At the conclusion of the incubation period in media containing Am80, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. Control cells are not detached, and are used directly in the differentiation assay as described in Example 2. Differentiation into Adipocytes and assessment of adipogenesis is carried out as described in Example 2.

For example, differentiation of the cell product into other cell types is achieved by reseeding the cells into differentiation media for adipocytes, or as described herein e.g., Examples 1, 2 and 81 to 84.

### Example 63

### Preparation of Cells Having the Ability to Differentiate into Other Cell Types by Incubation in Medium Comprising a Retinoid and Treatment with Protease: Method 6

In this set of experiments for producing cells having the ability to differentiate into different cell types, fibroblasts are cultured and then either incubated in media with or without BMS188649 and then are either incubated in media without trypsin or containing trypsin. The cells produced by this method are then tested for their ability to differentiate into adipocytes, as determined by the accumulation of fat.

### Preparation of BMS188649

Stock solution of BMS188649 (Bristol-Myers Squibb, Buffalo, N.Y.) are prepared by dissolving initially in dimethyl sulfoxide (DMSO) and then in ethanol, and finally in DMEM at a concentration of 10⁻⁵M for use at a final concentration of about 10⁻⁸M to 10⁻⁶M such that the final diluent concentration of ethanol is 0.1% (v/v) or less in all experiments. Stock solutions are prepared fresh for each experiment.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10⁻⁹ M to about 10⁻⁶M final concentration of BMS188649 (Bristol-Myers Squibb) dissolved ethanol for about 72 hr to 168 hrs, i.e., for about 3 to about 7 days, with a change of medium every 48 hours. Control cells are incubated with the same medium as test cells without BMS188649 but in the same final diluent concentration of carrier i.e., ethanol.

At the conclusion of the incubation period in media containing BMS188649, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells into differentiation media for adipocytes, or as described herein e.g., Examples 1, 2 and 81 to 84.

### Example 64

### Preparation of Cells Having the Ability to Differentiate into Other Cell Types by Incubation in Medium Comprising a Retinoid and Treatment with Protease: Method 7

In this set of experiments for producing cells having the ability to differentiate into different cell types, fibroblasts are cultured and then either incubated in media with or without CD336/Am580 and then are either incubated in media without trypsin or containing trypsin. The cells produced by this method are then tested for their ability to differentiate into adipocytes, as determined by the accumulation of fat.

### Preparation of CD336/Am580

Stock solution of CD336/Am580 (e.g., Galderma Laboratories, Sophia, France) are prepared by dissolving initially in dimethyl sulfoxide (DMSO) and then in ethanol, and finally in DMEM at a concentration of about 10⁻⁵M for use at a final concentration of about 10⁻⁸M to about 10⁻⁶M such that the final diluent concentration of ethanol is 0.1% (v/v) or less in all experiments. Stock solutions are prepared fresh for each experiment.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10⁻⁹M to about 10⁻⁶M final concentration of CD336/Am580 (Galderma Laboratories) dissolved ethanol for about 72 hr to 168 hrs, i.e., for about 3 to about 7 days, with a change of medium every 48 hours. Control cells are incubated with the same medium as test cells without CD336/Am580 but in the same final diluent concentration of carrier i.e., ethanol.

At the conclusion of the incubation period in media containing CD336/Am580, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells into differentiation media for adipocytes, or as described herein e.g., Examples 1, 2 and 81 to 84.

### Example 65

### Preparation of Cells Having the Ability to Differentiate into Other Cell Types by Incubation in Medium Comprising a Retinoid and Treatment with Protease: Method 8

In a this set of experiments for producing cells having the ability to differentiate into different cell types, fibroblasts are cultured and then either incubated in media with or without AGN193109 and then are either incubated in media without trypsin or containing trypsin. The cells produced by this method are then tested for their ability to differentiate into adipocytes, as determined by the accumulation of fat.

### Preparation of AGN193109

Stock solution of AGN193109 (e.g., Galderma Laboratories, Sophia, France) are prepared by dissolving initially in dimethyl sulfoxide (DMSO), and finally in DMEM at a concentration of 10⁻³ M for use at a final concentration of about 10⁻⁸ M to 10⁻⁶ M such that the final diluent concentration of ethanol is 0.1% (v/v) or less in all experiments. Stock solutions are prepared fresh for each experiment.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10 nM to about 1 μM final concentration of AGN193109 (Galderma Laboratories) dissolved ethanol for about 24 hr to 168 hrs, i.e., for about 1 day to about 7 days, with a change of medium every 48 hours. Control cells are incubated with the same medium as test cells without AGN193109 but in the same final diluent concentration of carrier i.e., DMSO.

At the conclusion of the incubation period in media containing AGN193109, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0 to 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells into differentiation media for adipocytes, or as described herein e.g., Examples 1, 2 and 81 to 84.

### Example 66

### Preparation of Cells Having the Ability to Differentiate into Other Cell Types by Incubation in Medium Comprising a Retinoid and Treatment with Protease: Method 9

The inventor sought to test whether or not incubating cells with at least two retinoid may produce equivalent or improved results as incubation with a single retinoid. In this set of experiments for producing cells having the ability to differentiate into different cell types, fibroblasts are cultured and then either incubated in media with or without Am80 and BMS188649 and then are either incubated in media without trypsin or containing trypsin. The cells produced by this method are then tested for their ability to differentiate into adipocytes, as determined by the accumulation of fat.

Production of Cells Capable of Differentiating into Other Cell Types

Am80 and BMS188649 are prepared as described in Example 62 and in Example 63, respectively. Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium High Glucose (DMEM-HG) and about 10⁻⁹ M to 10×10⁻⁶ M final concentration of Am80 (Galderma Laboratories) dissolved ethanol and with about 10⁻⁹ M to 10⁻⁶ M final concentration of BMS188649 (Bristol-Myers Squibb) dissolved ethanol for about 74 hr to 168 hrs, i.e., for about 3-7 days, with a change of medium every 48 hours. Control cells are incubated with the same medium as test cells without Am80 and BMS18 but in the same final diluent concentration of carrier i.e., ethanol.

At the conclusion of the incubation period in media containing Am80 and BMS18, cells are washed in PBS and the medium is replaced with growth medium Dulbecco's Modified Eagle Medium High Glucose (DMEM-HG; e.g., Lonza Cat #12-604) supplemented with 0% to 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 0% to 10% FBS) and maintained in this medium until required for re-differentiation. Control cells are not detached, and are used directly in the differentiation assay as described in Example 2. Differentiation into adipocytes and assessment of adipogenesis is carried out as described in Example 2.

For example, differentiation of the cell product into other cell types is achieved by reseeding the cells into differentiation media for adipocytes, or as described herein e.g., Examples 1, 2 and 81 to 84.

### Example 67

### Preparation of Cells Having the Ability to Differentiate into Other Cell Types by Incubation in Medium Comprising a Retinoid and Treatment with Protease, with Additional Incubation in Low-Serum

The inventor sought to test whether or not the additional step of incubating cells in a low serum media may produce equivalent or improved results as the combined action of incubation with a retinoid and incubation with a protease or alternatively by agonism of the Akt/(PKB) and/or the NF-κB pathway using an agonist compound. Without being bound by any theory or mode of action, the inventors reasoned that low-serum incubation conditions for 5-9 days further induces/enhances activation of the Akt/(PKB) and/or the NF-κB pathway. A possible advantage of using low-serum incubation for 5-9 days in concert together with incubation with a retinoid and detachment of cells e.g., by incubation with a protease such as trypsin and/or inducing the Akt/(PKB) and/or the NF-κB pathway using an agonist compound, is an increase in proportion of cells achieving optimum plasticity and/or enhanced survival under incubation conditions with a a retinoid.

Production of Cells Capable of Differentiating into Other Cell Types

Fresh human dermal fibroblasts derived from adult skin or from foreskin are are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per mm² surface area. Once all cells are attached, the medium is replaced with medium 199 (M199) (e.g., Sigma) supplemented with 0-1% FBS (low-serum) for different periods of time, from 1 to 11 days.

The medium is then replaced with M199 supplemented with 0-1% FBS (low-serum) and 10⁻⁸M to 10⁻⁶ M final concentration of ATRA for about 72 hr to 168 hrs, i.e., for about 3-7 days, as described in Example 60. Alternatively, the medium is replaced with M199 supplemented with 0-1% FBS (low-serum) and 10⁻⁸M to 10⁻⁶M final concentration of 9CRA for about 72 hr to 168 hrs, i.e., for about 3-7 days, as described in Example 61. Alternatively, the medium is replaced with M199 with 0-1% FBS (low-serum) and 10⁻⁹ M to 10×10⁻⁶ M final concentration of Am80 for about 72 hr to 168 hrs, i.e., for about 3-7 days, as described in Example 62. Alternatively, the medium is replaced with M199 supplemented with 0-1% FBS (low-serum) and 10⁻⁹M to 10⁻⁶M final concentration of BMS188649 for about 72 hr to 168 hrs, i.e., for about 3-7 days, as described in Example 63. Alternatively, the medium is replaced with M199 supplemented with 0-1% FBS (low-serum) and 10⁻⁹M to 10⁻⁶M final concentration of CD336/Am580 for about 72 hr 20 to 168 hrs, i.e., for about 3-7 days, as described in Example 64. Alternatively, the medium is replaced with M199 supplemented with 0-1% FBS (low-serum) and 10 nM to 1 μM final concentration of AGN193109 for about 24 hr to 168 hrs, i.e., for about 1 day to 7 days, as described in Example 65. Alternatively, the medium is replaced with M199 supplemented with 0-1% FBS (low-serum) and 10⁻⁹M to 10×10⁻⁶M final concentration of Am80 and with 10⁻⁹M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

At the conclusion of the incubation period in low serum media, test cells are detached by the addition of 20 μl of detachment solution containing 0.12% trypsin, 0.02% EDTA and 0.04% glucose and incubated at 37° C. until cells lifted from the plates. Test cells are recovered from culture, then diluted to 200 μl with serum-free DMEM-HG (0% FBS) and maintained in serum-free medium until required for re-differentiation. Control cells are not detached, and are used directly in the differentiation assay as described in Example 2. Differentiation into Adipocytes and assessment of adipogenesis is carried out as described in Example 2. The person skilled in the art would appreciate that differentiation of test cells into adipocytes may continue albeit at below optimum even after the 11-day period incubation at low serum.

For example, differentiation of the cell product into other cell types is achieved by reseeding the cells into differentiation media for adipocytes, or as described herein e.g., Examples 1, 2 and 81 to 84.

### Example 68

### Preparation of Cells Having the Ability to Differentiate into Other Cell Types by Incubation in Medium Comprising a Retinoid and Treatment with Protease and Additional Incubation at High Cell Density Conditions

To improve the yield of cells having the ability to differentiate into other cell types, the inventor sought to investigate the effect of high density cultures on plasticity. Specifically, the inventor sought to test whether or not the additional step of incubating cells at high density in a suitable differentiation media may produce equivalent or improved results as the combined action of incubation with a retinoid and incubation in the presence of protease or alternatively by agonism of the Akt/(PKB) and/or the NF-κB pathway using an agonist compound. Without being bound by any theory or mode of action, the inventor reasoned that culturing protease treated cells at high cell density in a high density plating medium, further induces activation of the NF-κB pathway. A possible advantage of using a high cell density following protease treatment to induce the NF-κB pathway, in concert with incubating cells with a retinoid and in the presence of a protease and/or inducing the Akt/(PKB) and/or the NF-κB pathway using an agonist compound, is an increase in proportion of cells achieving optimum plasticity.

In this set of experiments for producing cells having the ability to differentiate into different cell types, fresh human dermal fibroblasts derived from adult skin or from foreskin fibroblasts are cultured and detached by incubation with trypsin essentially as described in any one of examples 58 to 66.

Test cells are then recovered from culture immediately after trypsinization and are diluted to about 100,000 cells in 100 p. 1 in high density plating medium (e.g., Medium 199 containing 170 nM insulin, 0.5 mM 3-isobutyl-1-methylxanthine, 0.2 mM indomethacin, 1 μM dexamethasone, and 15% rabbit serum). Within about 4 to 6 hours after trypsinization, test cells are recovered from culture and seeded at concentrations of about 100,000 cells per well/plate or at about 3703.7 cells per mm² surface area of the well/plate before attachment of the cells to the plate/well directly in 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum) for a time and under conditions sufficient for an optimum number of progenitor cells to be produced e.g., for up to about 24 hours or until adherence is achieved i.e., a shorter time than required for cells to become adherent and/or as determined by analysis of cell marker expression and/or by the ability of aliquots of cells to subsequently undergo differentiation.

As a negative control for the production of progenitor cells, trypsinized cells are seeded at a reduced density i.e., about 740.1 cells per mm² surface area, in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and incubated as for samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation.

Differentiation into Adipocytes

For differentiation into adipocytes, cells are incubated in adipogenic medium (Medium 199 containing 170 nM insulin, 0.5 mM 3-isobutyl-1-methylxanthine, 0.2 mM indomethacin, 1 μM dexamethasone, and 15% rabbit serum) at high density and allowed to expand for about 10-21 days.

As a negative control for differentiation, trypsinized cells are seeded at high density in high density plating medium (e.g., DMEM-HG supplemented with 10% FBS) and incubated as for test samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation. The high density plating medium is then replaced with 200 to 400 μl DMEM-HG (10% FCS) medium and cells are allowed to expand for about 10-21 days.

As positive control for differentiation, rat bone marrow stromal/stem cells (rBMSCs) are expanded in DMEM medium containing L-Glutamine and 10% FCS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 400 μl DMEM-HG containing 10% FCS for up to about 24 hours or until adherent. The medium is replaced from adherent culture with 200 to 400 μl adipogenic medium (Medium 199 containing 170 nM insulin, 0.5 mM 3-isobutyl-1-methylxanthine, 0.2 mM indomethacin, 1 μM dexamethasone, and 15% rabbit serum) and cells are allowed to expand for about 10-21 days.

Medium is replaced every 3 days for both test cells and negative and positive control cells.

### Assessment of Adipogenesis

After incubation for 12-21 days in adipogenic medium, differentiation potential of test cells compared to control cells at each day of incubation at high density post incubation optionally with low-serum and trypsinization is measured by an assessment of adipogenesis as described in example 2.

### Example 69

### Preparation of Cells Having the Ability to Differentiate into Other Cell Types by Induction of the Akt/(PKB) Pathway: Method 1

The data in Examples 58-66 suggest to the inventor that agonism of the Akt/(PKB) pathway and/or NF-κB pathway may produce equivalent or improved results as the combined action incubation in the presence of a retinoid and incubation in the presence of a protease such as trypsin to detach cells. Without being bound by any theory or mode of action, the inventor reasoned that incubation with a retinoid and detachment of the cells to induce optimum plasticity of fibroblasts coincided with the induction of the Akt/(PKB) pathway, and that the responses of cells to the combined retinoid and trypsinization conditions is likely to induce the Akt/(PKB) pathway. Accordingly, the inventor sought to test whether or not incubation with a retinoid and incubation in the presence of a protease such as trypsin could be reproduced or improved upon by incubation in the presence of one or more agonists of the Akt/(PKB) pathway. A possible advantage of using an agonist to induce the Akt/(PKB) pathway, as opposed to incubating cells a retinoid followed by trypsinization, or in concert with such a process, is enhancement of cell survival and the proportion of cells achieving optimum plasticity. By enhancing induction of the Akt/(PKB) pathway using an agonist compound, differentiated primary cells and cell lines that would normally enter a quiescent state or undergo apoptosis as a result of exposure to a retinoid can be used to produce cells capable of differentiating into different cell types.

In one example to show that Akt/(PKB) pathway induction confers or enhances plasticity of fibroblasts, primary human foreskin fibroblasts are incubated in the presence of human recombinant PDGF-BB for a time and under conditions sufficient to induce the Akt/(PKB) pathway.

Production of Cells Capable of Differentiating into Different Cell Types

Fresh human dermal fibroblasts derived from adult skin or from foreskin are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA (low-protein) for 24 hours to precondition the cells for PDGF-BB. After 24 hours, the medium is replaced with low-serum or serum-free DMEM containing 10 to 100 ng/ml of human recombinant PDGF-BB (Invitrogen) for 5 to 15 min to activate the Akt/(PKB) pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹ M to 10⁻⁶ M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 (Bristol-Myers Squibb) for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (e.g., Lonza) (0% to 10% FBS) and maintained in this medium until required for re-differentiation.

Differentiation into Other Cell Types

Re-differentiation of the treated fibroblasts into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 70

### Preparation of Cells Having the Ability to Differentiate into Other Cell Types by Induction of the Akt/(PKB) Pathway: Method 2

In a further example to show that Akt/(PKB) pathway induction confers or enhances plasticity of fibroblasts, primary fibroblasts are incubated in the presence of TGF-β for a time and under conditions sufficient to induce the Akt/(PKB) pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or bovine serum albumin (BSA) (low-protein) for 24 hours to precondition the cells for TGF-β. After 24 hours, the medium is replaced with serum-free or low-serum DMEM-HG (e.g., Lonza) containing 1 to 10 ng/ml of TGF-β (R&D systems) for at least 60 min to activate the Akt/(PKB) pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (e.g., Lonza) (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 71

### Preparation of Cells Having the Ability to Differentiate into Other Cell Types by Induction of the Akt/(PKB) Pathway: Method 3

In a further example to show that Akt/(PKB) pathway induction confers or enhances plasticity of fibroblasts, primary fibroblasts are incubated in the presence of sodium pyruvate for a time and under conditions sufficient to induce the Akt/(PKB) pathway.

Production of Cells Capable of Differentiating into Different Cell Types

Fresh human dermal fibroblasts derived from adult skin or from foreskin are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or bovine serum albumin (BSA) (low-protein) for 24 hours to precondition the cells for sodium pyruvate. After 24 hours, the medium is replaced with serum-free or low-serum DMEM-HG containing 50 to 200 mg/L of cell culture grade sodium pyruvate (e.g., Lonza), and preferably, at 110 mg/L for at least 1 h to activate the Akt/(PKB) pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹ to 10⁻⁶ M final concentration of BMS188649 for about 72 hr to 168 hrs, i.e., for about 3-7 days, as described in Example 63. Alternatively, the medium is replaced with DMEM-HG and about 10⁻⁹ M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG with sodium pyruvate (e.g., Lonza Cat #12-604) (0% to 10% FBS) and maintained in serum-free medium until required for re-differentiation.

Re-differentiation of the treated fibroblasts into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 72

### Preparation of Cells Having the Ability to Differentiate into Other Cell Types by Induction of the Akt/(PKB) Pathway: Method 4

In a further example to show that Akt/(PKB) pathway induction confers or enhances plasticity of fibroblasts, mouse dermal primary fibroblasts are incubated in the presence of PDGF-BB for a time and under conditions sufficient to induce the Akt/(PKB) pathway.

Production of Cells Capable of Differentiating into Different Cell Types

Mouse dermal fibroblast cells are prepared from 8-12 week-old C57BL/6 mice. Briefly, mice are anesthetized with pentobarbital (50 mg/kg body weight), and a full thickness of the back skin is cut out by scissors. The skin tissues are cut into small pieces and are implanted into plastic tissue culture dishes containing DMEM-HG (e.g., Lonza) with 10% FBS. The fibroblast cultures are used after three to seven passages.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶M final concentration of BMS188649 for about 72 hr to 168 hrs, i.e., for about 3-7 days, as described in Example 63. Alternatively, the medium is replaced with DMEM-HG and about 10⁻⁹ M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the treated fibroblasts into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 73

### Preparation of Cells Having the Ability to Differentiate into Other Cell Types by Induction of the Akt/(PKB) Pathway: Method 5

In a further example to show that Akt/(PKB) pathway induction confers or enhances plasticity of cells generally, rat adrenal cells are incubated in the presence of Carbachol or NGF for a time and under conditions sufficient to induce the Akt/(PKB) pathway.

Production of Cells Capable of Differentiating into Different Cell Types

PC12 cells are obtained from the American Type Culture Collection (CRL-1721, Rockville, Md.). PC12 cells are cultured in DMEM-HG supplemented with 5% (v/v) fetal calf serum and 10% (v/v) heat-inactivated horse serum, and grown at 37° C. in an environment of 7.5% CO₂ as described previously (Yu et al, Neurosignals 13: p248 (2004).

Adherent PC12 cultures are incubated in DMEM supplemented with 0-1% FBS or bovine serum albumin (BSA) (low-protein) for 24 hours to precondition the cells for Carbachol or NGF. After 24 hours, the medium is replaced with serum-free DMEM-HG or low-serum DMEM containing 200-1000 μM Carbachol (Calbiochem) or at least 50 ng/ml purified NGF (2.5S) (Alomone Labs Ltd) for 5 to 10 min to activate the Akt/(PKB) pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶ M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 74

### Preparation of Cells Having the Ability to Differentiate into Other Cell Types by Induction of the Akt/(PKB) Pathway: Method 6

In a further example to show that Akt/(PKB) pathway induction confers or enhances plasticity of cells generally, embryo fibroblasts are incubated in the presence of insulin growth factor-1 (IGF-1) for a time and under conditions sufficient to induce the Akt/(PKB) pathway.

Production of Cells Capable of Differentiating into Different Cell Types

Non-transformed rat embryo fibroblasts (Rat-1) are prepared and maintained as previously described (Peterson, et al., J. Biol. Chem. 271:31562-31571 (1996)).

Adherent Rat-1 cultures are incubated in DMEM-HG supplemented with 0-1% FBS or bovine serum albumin (low-protein) for 12 hours to precondition the cell for IGF-1. After 12 hours, the medium is replaced with serum-free DMEM or low-serum DMEM containing at least 250 ng/ml of insulin growth factor-1 (IGF-1; Sigma) for at least about 20 min to activate the Akt/(PKB) pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of for about 72 hr to 168 hrs, i.e., for about 3-7 days, as described in Example 62. Alternatively, the medium is replaced with DMEM-HG and about 10⁻⁹M to 10⁻⁶M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 75

### Preparation of Cells Having the Ability to Differentiate into Other Cell Types by Induction of the NF-κB Pathway: Method 1

The data in Examples 58-64 also suggested to the inventor that agonism of the NF-κB pathway may produce equivalent or improved results as the combined action of incubation in the presence of a retinoid and incubation in the presence of a protease such as trypsin to detach cells. Without being bound by any theory or mode of action, the inventor reasoned that incubation in the presence of a retinoid and detachment of the cells to induce optimum plasticity of fibroblasts coincided with induction of the NF-κB pathway, and that the responses of cells to the combined retinoid and trypsinization conditions is likely to induce the NF-κB pathway. Accordingly, the inventor sought to test whether or not the effect of incubation in the presence of a retinoid and incubation in the presence of a protease such as trypsin could be reproduced or improved upon by incubation in the presence of one or more agonists of the NF-xB pathway. A possible advantage of using an agonist to induce the NF-κB pathway, in concert with or as opposed to incubating cells with a retinoid followed by trypsinization, is enhancing the proportion of cells achieving optimum plasticity. By enhancing induction of the NF-κB pathway using an agonist, differentiated primary cells and cell lines that would normally enter a quiescent state or undergo apoptosis as a result of exposure to retinoid can be used to produce cells capable of differentiating into different cell types.

In one example to show that NF-κB pathway induction confers or enhances plasticity of cells generally, primary human dermal fibroblasts are incubated in the presence of TNF-α for a time and under conditions sufficient to induce the NF-κB pathway.

Production of Cells Capable of Differentiating into Different Cell Types

Fresh human dermal fibroblasts derived from adult skin or from foreskin are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with serum-free DMEM or low-serum DMEM-HG containing at least 20 ng/ml of TNF-α (Roche) for at least 60 min to activate the NF-κB pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹ M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶ M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 76

### Preparation of Cells Having the Ability to Differentiate into Other Cell Types by Induction of the NF-κB Pathway: Method 2

In a further example to show that NF-κB pathway induction confers or enhances plasticity of cells generally, primary human dermal fibroblasts are incubated in the presence of interleukin-1α for a time and under conditions sufficient to induce the NF-κB pathway.

Production of Cells Capable of Differentiating into Different Cell Types

Fresh human dermal fibroblasts derived from adult skin or from foreskin are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with DMEM supplemented with 0.25% FBS of BSA for 50 hours to precondition the cells to interleukin-1α. After 50 hours, the cells are treated with recombinant human IL-1α at a concentration of least 0.27 ng/ml to activate the NF-κB pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA (Sigma) for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Differentiation into Other Cell Types

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 77

### Preparation of Cells Having the Ability to Differentiate into Other Cell Types by Induction of the NF-κB Pathway: Method 3

In a further example to show that NF-κB pathway induction confers or enhances plasticity of fibroblasts, primary fibroblasts are incubated in the presence of sodium pyruvate for a time and under conditions sufficient to induce the NF-κB pathway.

Production of Cells Capable of Differentiating into Different Cell Types

Fresh human dermal fibroblasts derived from adult skin or from foreskin are plated in cell culture flasks, or plates, in growth medium (DMEM-HG without sodium pyruvate; for example Lonza Cat. #12-741) supplemented with 10% FBS (fetal bovine serum), and incubated at 37° C. in a humidified atmosphere of 5% CO₂ in air until adherent. Once all cells are attached, the medium is replaced with DMEM-HG without sodium pyruvate (e.g., Lonza) supplemented with 0-1% FBS or BSA (low-protein) for 24 hours to precondition the cells to sodium pyruvate treatment. After 24 hours, the medium is replaced with serum-free or low-serum DMEM-HG with sodium pyruvate containing 50 to 200 mg/L of cell culture grade sodium pyruvate (e.g., Lonza), and preferably, at 110 mg/L for at least 1 h to activate the NF-κB pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹ M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG with sodium pyruvate (e.g., Lonza Cat #12-604) (0% to 10% FBS) and maintained in this medium until required for re-differentiation.

Differentiation into Other Cell Types

Re-differentiation of the treated fibroblasts into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 78

### Preparation of Cells Having the Ability to Differentiate into Other Cell Types by Induction of the NF-κB Pathway: Method 4

In a further example to show that NF-κB pathway induction confers or enhances plasticity of cells generally, mouse embryo fibroblasts are incubated in the presence of L-alpha-Lysophosphatidic acid (C18:1, [cis]-9), LPA for a time and under conditions sufficient to induce the NF-κB pathway.

Production of Cells Capable of Differentiating into Different Cell Types

Swiss 3T3 mouse embryo fibroblasts are obtained from the American Type Culture Collection (CCL-92, Rockville, Md.) and are cultured at 37 C under a humidified atmosphere of 10% CO₂ in Dulbecco's modified Eagle's medium (DMEM) containing 10% (v/v) fetal calf serum.

Adherent 3T3 fibroblast cultures are incubated in DMEM-HG supplemented with 1% FBS or BSA (low-protein) for 18 hours to precondition the cells to L-alpha-Lysophosphatidic acid (C18:1, [cis]-9), LPA treatment. After 18 hours, L-α-Lysophosphatidic acid (C18:1,[cis]-9), LPA (Calbiochem; prepared as a stock of 1 mg/ml in phosphate-buffered saline containing 10 mg/ml essentially fatty acid-free bovine serum albumin (Sigma) is added to adherent cultures at 40-100 μM final concentration for about 40-120 min to activate the NF-κB pathway. As a control, TNF-α (Roche) is added to separate parallel cultures at a final concentration of 30 ng/ml for the same time period to activate the NF-κB pathway e.g., as described in Example 583.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA (Sigma) for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 firs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 79

### Preparation of Cells Having the Ability to Differentiate into Other Cell Types by Induction of the NF-κB Pathway: Method 5

In a further example to show that NF-κB pathway induction confers or enhances plasticity of cells generally, human myometrial microvascular endothelial cells (HUMEC) are incubated in the presence of Lipopolysaccharide (LPS) for a time and under conditions sufficient to induce the NF-κB pathway.

Production of Cells Capable of Differentiating into Different Cell Types

Human myometrial microvascular endothelial cells (HUMEC) are obtained from Technoclone GmbH (Vienna, Austria) and are cultured at 37 C in endothelial growth medium according to the specifications supplied by Technoclone GmbH.

Adherent HUMEC cultures are then incubated in endothelial medium, preferably serum free or containing low-serum concentration, and supplemented with 10-100 ng/ml of Lipopolysaccharide (LPS; Sigma) for at least 45 min to activate the NF-κB pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA (Sigma) for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹M to 10⁻⁶ M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 80

### Preparation of Cells Having the Ability to Differentiate into Other Cell Types by Induction of the NF-κB Pathway: Method 6

In a further example to show that NF-κB pathway induction confers or enhances plasticity of cells generally, synovial fibroblasts are incubated in the presence of Lipopolysaccharide (LPS) for a time and under conditions sufficient to induce the NF-κB pathway.

Production of Cells Capable of Differentiating into Different Cell Types

Primary cultures of synovial fibroblasts are obtained and maintained in culture as described previously (Brinckerhoff, and Mitchell, Journal of Cellular Physiology, 136 (1):72-80 (2005)).

Adherent synovial fibroblast cultures are then incubated in growth medium, preferably serum free or containing low-serum concentration, and supplemented with 10-100 ng/ml of Lipopolysaccharide (LPS; Sigma) for at least 45 min to activate the NF-κB pathway.

The medium is then replaced with M199 comprising retinoic acid at about 10⁻⁸ M final concentration of for about 72 hr to 168 hrs, i.e., for about 7-9 days, as described in Example 58. Alternatively, the medium is replaced with DMEM-HG supplemented with about 5% to about 50% FBS (fetal bovine serum) comprising retinoic acid at final concentration of about 0.255×10⁻⁷ M to about 0.255×10⁻⁶ M for about 5-7 days, as described in Example 59. Alternatively, the medium is then replaced with DMEM-HG and about 10⁻⁸ M to 10⁻⁶ M final concentration of ATRA (Sigma) for about 72 hr to 168 hrs, i.e., for about 3-7 days, as described in Example 60. Alternatively, the medium is replaced with DMEM-HG and about 10⁻⁸ M to 10⁻⁶ M final concentration of 9CRA for about 72 hr to 168 hrs, i.e., for about 3-7 days, as described in Example 61. Alternatively, the medium is replaced with DMEM-HG and about 10⁻⁹M to 10×10⁻⁶ M final concentration of Am80 for about 72 hr to 168 hrs, i.e., for about 3-7 days, as described in Example 62. Alternatively, the medium is replaced with DMEM-HG and about 10⁻⁹ M to 10⁻⁶ M final concentration of BMS188649 for about 72 hr to 168 hrs, i.e., for about 3-7 days, as described in Example 63. Alternatively, the medium is replaced with DMEM-HG and about 10⁻⁹M to 10⁻⁶ M final concentration of CD336/Am580 for about 72 hr to 168 hrs, i.e., for about 3-7 days, as described in Example 64. Alternatively, the medium is replaced with DMEM-HG and about 10 nM to 1 μM final concentration of AGN193109 for about 24 hr to 168 hrs, i.e., for about 1 day to 7 days, as described in Example 65. Alternatively, the medium is replaced with DMEM-HG and about 10⁻⁹ M to 10×10⁻⁶ M final concentration of Am80 and with 10⁻⁹ M to 10⁻⁶ M final concentration of BMS188649 for about 74 hr to 168 hrs, i.e., for about 3-7 days, as described in Example 66. Control cells are incubated with the same medium as test cells without any retinoid but with 0.1% ethanol or DMSO carrier.

Preferably, treated adherent cells are detached from larger plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (0% to 10% FBS) and maintained in this medium until required for re-differentiation.

Re-differentiation of the cell product into other cell types is achieved by reseeding the treated cells described above into differentiation media, preferably after trypsinization and before reattachment. Methods suitable for differentiation of these cells into other cell types are known in the art and described herein e.g., Examples 1, 2 and 81 to 84.

### Example 81

### Differentiation of Cells into Cells of Osteogenic Lineage

This example describes methods for producing cells of osteogenic lineage from the cell product of any one of Examples 22 through to 80 that is capable of being differentiated into a different cell type. This example also describes methods for testing that osteogenic cells are produced.

### Differentiation Conditions

Cells capable of producing other cell types are prepared as in any one of Examples 1 to 17 and counted.

To produce cells of osteogenic lineage from such cells, the cells are incubated in complete osteogenic media (+DEX: DMEM-low glucose containing 10% FBS, 20 μg/ml ascorbic acid phosphate-magnesium salt, 1.5 mg/ml beta glycerophosphate and 40 ng/ml dexamethasone) for differentiation to the osteogenic lineage, or in incomplete osteogenic media (−DEX: DMEM-low glucose containing 10% FBS, 20 μg/ml ascorbic acid phosphate-magnesium salt, 1.5 mg/ml beta glycerophosphate), as a control. The cells are then plated in their respective media onto 96-well plates at about 20,000 cells per well or about 740.74 cells per mm² surface area of the well for alkaline phosphatase assays (ALP) or at 50,000 cells per well or about 1851.85 cells per mm² surface area of the well for mineral deposition assays as described below. Alternatively, the cells are plated in their respective differentiation media onto 96-well plates at about 100,000 cells per well or at about 3703.7 cells per mm² surface area of the well where optional high density plating step is employed e.g., as in Example 5. Complete or incomplete osteogenic media is replaced every 3 days.

### Assessment of Osteogenesis Using an Alkaline Phosphatase (ALP) Assay

After incubation for 12-21 days in either complete or incomplete osteogenic media as described above, alkaline phosphatase is assessed. The media is removed from cells; cells are washed in phosphate buffered saline and lysed with 40 μl of Passive Lysis Buffer (Promega). The lysate is sonicated. After sonication, the lysate is split into two equal samples of 20 μl, each. One sample is placed into a separate 48 well plate, Add 180 μL of Hoescht 33258 in buffer (5 μg/mL in 2M NaCl or 20×SSC) (i.e 1:9 ratio of PLB to Hoescht) is added, and the sample is read at Excitation 350 nm/Emission 460 on Molecular Probes fluorescent scanner. p-Nitrophenyl phosphate (pNPP) 75 μL is added to the remaining sample and incubated for 30 minutes at 37° C. One hundred (100) μl of 2M NaOH is subsequently added which will turn into yellow p-Nitrophenylene anion-pNP. An aliquot of 100 μl is transferred to a 96 well plate for plate reading. The absorbance of pNP (yellow) is read on an optical plate reader at 405 nm. A comparison of +Dex to −Dex controls of Absorbance/ng DNA using a PNPP standard curve is made.

### Assessment of Mineral Deposition

After incubation for 21 days in either complete or incomplete osteogenic media as described above, mineral deposition is assessed. To test for mineral deposition, cells are stained with Von Kossa. A comparison of staining intensity is performed on +Dex differentiated cells to −Dex treated controls.

### Example 82

### Differentiation of Cells into Cells of Chondrogenic Lineage

This example describes methods for producing cells of chondrogenic lineage from the cell product of any one of Examples 22 through 80 that is capable of being differentiated into a different cell type. This example also described methods for testing that chondrogenic cells are produced.

### Differentiation Conditions

Cells capable of producing other cell types are prepared as in any one of Examples 22 to 80 and counted.

To produce cells of chondrogenic lineage from such cells, the cells incubated in chondrogenic media (DMEM-HG containing ITS+supplement at a 1 fold concentration (final concentrations of 6.25 μg/ml bovine insulin; 6.25 μg/ml transferrin; 6.25 μg/ml selenous acid; 5.33 μg/ml linoleic acid; 1.25 mg/ml BSA) 50 μs/ml ascorbic acid-2-phosphate, 40 μg/ml L-proline, 100 μg/ml pyruvate, 100 nM dexamethasone, 10 ng/ml TGF-(3, and 500 ng/ml BMP-2) for differentiation to the chondrogenic lineage; or in DMEM-HG containing 1.25 mg/ml BSA, as a control. The cells are then plated in their respective media onto 96-well plates at about 20,000-50,000 cells per well or at about 740.74-1851.85 cells per mm² surface area of the well. Alternatively, the cells are plated in their respective differentiation media onto 96-well plates at about 100,000 cells per well or at about 3703.7 cells per mm² surface area of the well where optional high density plating step is employed. Chondrogenic media or control media is replaced every 3 days.

### Assessment of Chondrogenesis

After incubation for 12-21 days in either chondrogenic media or control media as described above, cells are assessed by observation for the appearance of chondrocyte morphology. Analysis of the accumulation of sulfated glycosaminoglycans (GAG) is carried out by measuring the amount of 1,9-dimethylmethylene blue-reactive material in extracts of cells treated with chondrogenic media and compared with extracts of control cells. The 1,9-dimethylmethylene blue assay is performed essentially as described in Sabiston et al, Analytical Biochemistry 149: 543-548 (1985) incorporated herein by reference in its entirety.

### Example 83

### Differentiation of Cells into Haematopoietic Cells

This example describes methods for producing haematopoietic cells from the cell product of any one of Examples 22 through 80 that is capable of being differentiated into a different cell type. This example also described methods for testing that haematopoietic cells are produced.

### Differentiation Conditions

Cells capable of producing other cell types are prepared as in any one of Examples 22 to 80 and counted.

To produce haematopoietic cells from such cells, the cells are mixed with DMEM supplemented with Granulocyte macrophage colony-stimulating factor (GM-CSF; 50 ng/ml) and stem cell factor (SCF; 50 ng/ml), plated onto 35-mm tissue culture dishes and are incubated at 37° C. in a humidified atmosphere of 5% CO₂ in air for 2 days. The cells are harvested and analyzed for cells expressing the hematopoietic marker CD45 by flow cytometry.

To detect the presence of the cell surface CD45 antigen, cells are incubated for 30 min. at 37° C. with anti-CD45 antibodies (Becton Dickinson), washed in PBS and analysed by flow cytometry. Flow cytometric analysis is performed using a FACSCalibur flow cytometer and the CellQuest software program (Becton Dickinson Immunocytometry Systems, San Jose, Calif.). Data analysis is performed using CellQuest and the Modfit LT V2.0 software program (Verity Software House, Topsham, Me.).

### Example 84

### Differentiation of Cells into Insulin-Secreting Cells

This example describes methods for producing insulin-secreting cells from the cell product of any one of Examples 22 through 80 that is capable of being differentiated into a different cell type. This example also described methods for testing that insulin-secreting cells are produced.

### Differentiation Conditions

Cells capable of producing other cell types are prepared as in any one of Examples 22 to 80, and counted.

To produce insulin-secreting cells from such cells, the cells are plated into serum free medium to enrich for nestin-positive cells (see Lumelsky et al., Science, 292:1389, 2001). The nestin-positive cells are then sub-subcultured and expanded for 6 to 7 days in serum-free N2 media supplemented with 1 μg/ml laminin, 10 ng/ml bFGF, 500 ng/ml N-terminal fragment of murine or human SHH (sonic hedge hog) 100 ng/ml FGF8 and B27 media supplement, as described in Lee et al. Nature Biotechnology, 18: 675 (2000) and Lumelsky (supra), which are herein incorporated by reference. After the nestin-positive cells are expanded, the growth factors (FGF, SHH) are removed from the media and nicotinamide is added to the media at a final concentration of 10 mM, to promote the cessation of cell proliferation and induce the differentiation of insulin-secreting cells. After approximately 6 days of growth factor starvation, aggregates of insulin-secreting cells are formed (islet-like cell clusters), which are autologous to the individual from whom they are derived.

### Example 85

### Multipotency of Cells Produced in Accordance with the Invention

### Retinoic Acid-Induced Differentiation of the Cells

Cells are tested for an ability to regenerate their telomeres, as determined by expression of telomerase. The expression of relatively high levels of telomerase in a cell culture is indicative of a stem cell-like phenotype. Furthermore, retinoic acid (RA)-induced differentiated cells down-regulate the expression of telomerase and express genes indicative of differentiating cells of various lineages. For example, Schuldiner et al., PNAS 97:11307 (2000) demonstrated the increased expression of tissue specific lineage markers, e.g., brain-specific neurofilament (ectodermal), heart-specific cardiac actin (mesodermal) and liver-specific α1-antitrypsin (endodermal), in cultures of human embryonic stem cells treated with RA.

Cells prepared as described in any one of Examples 1 through 80 hereof are cultured in the presence of approximately 1-2 μM RA (Sigma, St. Louis) for 5 to 10 days, preferably under high cell density incubation conditions preferably before reattachment in high density plating medium and/or in the presence of one or more agonists of the Akt/(PKB) pathway and/or NF-κB pathway to maintain their plasticity.

### RNA Extraction and RT-PCR

To monitor the differential expression of various genes in the cells, reverse transcription-polymerase chain reaction (RT-PCR) is performed. RNA is extracted from untreated cells and cells treated with RA, e.g., using Perfect RNA™ Eukaryotic Kit (Eppendorf A G, Hamburg, D E), essentially according to the manufacturer's instructions. The extracted RNA is dissolved in RNase-free water e.g., provided in the Perfect RNA™ Eukaryotic Kit.

RT-PCR is performed using the QIAGEN® OneStep RT-PCR Kit (Qiagen Inc., Valencia, Calif.) according to the manufacturer's instructions. PCR amplification is preformed using the following protocol: 94° C. for 1 min., 55° C. for 1 min., 72° C. for 1 min., for 45 cycles.

The oligonucleotide primers set out below are used to detect the following mRNAs: human telomerase (“TRT”), neurofilament heavy chain (“NF”), alpha-antitrypsin (“aAT”) and cardiac actin (“cACT”). To control for the quality of the extracted RNA and to serve as an internal quantification marker, human glyceraldehyde 3-phosphate dehydrogenase (“GAPDH”) oligonucleotide primers are included in the RT-PCR reaction.

RT-PCR primer sets:

| | |
|---|---|
| GAPDH | 5′-GGGGAGCCAAAAGGGTCATCATCT-3-′; |
| | 5′-GACGCCTGCTTCACCACCTTCTTG-3′ |
| TRT | 5′-CGGAGGTCATCGCCAGCATCATCA-3-′ |
| | 5′-GTCCCGCCGAATCCCCGCAAACAG-3′ |
| NF | 5′-TGAACACAGACGCTATGCGCTCAG-3′ |
| | 5′-CACCTTTATGTGAGTGGACACAGAG-3′ |
| αAT | 5′-AGACCCTTTGAAGTCAAGGACACCG-3′ |
| | 5′-CCATTGCTGAAGACCTTAGTGATGC-3′ |
| cACT | 5′-TCTATGAGGGCTAGCCTTTG-3′ |
| | 5′-CCTGACTGGAAGGTAGATGG-3′ |

The RT-PCR products are electrophoresed on 2% (w/v) agarose gels stained with ethidium bromide. The intensities of the DNA product bands are quantified e.g., using PHORETIX™ TotalLab densitometry software package developed by Nonlinear USA (Durham, N.C.). To determine the approximate relative percent change in the expression of TRT, NF, aAT and cACT in each of the experimental groups relative to the untreated fibroblasts the following equation is applied (Eq. 1):

x=([(a′/b′)/(a/b)]−1)100% Eq. 1

wherein x is the relative percent change in expression of the gene of interest; b is the intensity of the GAPDH band in untreated fibroblasts; b′ is the intensity of the GAPDH band obtained from the experimental cells; a is the intensity of the gene-of-interest band obtained from the untreated fibroblasts; and a′ is the intensity of the gene-of-interest band obtained from the experimental cells.

Cells that are ectodermal-like, or mesodermal-like or endodermal-like can be differentiated into specialized tissues normally derived from each embryonic layer and subsequently used for treatment and/or therapy of disease.

### Example 86

### Therapy Using Differentiated Cells Produced in Accordance with the Invention

This example describes therapeutic applications of progenitor cells produced from differentiated cells in accordance with the inventive method.

### Diabetes

To treat human patients suffering from diabetes, progenitor cells are produced from differentiated cells and then differentiated into insulin-secreting cells as described in Example 6. Preferably, the differentiated cells used as starting material in this process are derived from the same patient or a matched patient to minimize or eliminate the risk of graft rejection. The insulin-secreting cells are grafted subcutaneously into a subject suffering from diabetes, wherein the cells are either encapsulated in a polymer matrix or non-encapsulated and containing a suitable isotonic buffer, or surgically infused into the patient's pancreas. A therapeutic amount of insulin-secreting cells are implanted in the patient subcutaneously. The skilled practitioner may determine a therapeutic amount based upon the age, weight and general health of the patient and the amount of insulin secreted by said insulin-secreting cells in response to glucose administration. Blood glucose levels of the patient are monitored on a regular basis and the amount of implanted cells are adjusted accordingly.

### Osteoarthritis

To treat human patients suffering from degenerative osteoarthritis, progenitor cells are produced from differentiated cells and then differentiated into chondrocytes as described in Example 4. Preferably, the differentiated cells used as starting material in this process are derived from the same patient or a matched patient to minimize or eliminate the risk of graft rejection. Cells of chondrocyte lineage are grafted into the diseased joints of a patient by implantation with a needle, or by orthoscopic surgical methods, wherein the cells are either encapsulated in a polymer matrix or non-encapsulated and containing a suitable isotonic buffer. Again, a therapeutic amount of chondrocytes are implanted in the patient's degenerated joints. The skilled practitioner may determine a therapeutic amount based upon the age, weight and general health of the patient and the disease progression in the patient.

1. A method for producing a progenitor cell capable of being differentiated into a plurality of different cell types, said method comprising incubating culturing differentiated cells and detaching the cells, wherein said method produces progenitor cells capable of being differentiated into a plurality of different cell types.

2. The method of claim 1, comprising producing progenitor cells capable of being differentiated into a plurality of different cell types until re-attachment or adherence or contact of the cells to the culture vessel and/or to each other.

3. The method of claim 1, wherein detaching the cells is capable of inducing trans-differentiation of the differentiated cells into the progenitor cells.

4. The method according to claim 1, wherein said method further comprises incubating or maintaining or culturing the cells in high cell-density conditions.

5. The method according to claim 1, wherein incubating or maintaining or culturing the cells in high cell-density conditions comprising incubating or maintaining or culturing the cells until confluence or cell-to-cell contact is achieved.

6. The method according to claim 1, wherein the high cell-density conditions comprise a minimum density between about 1500 cells/mm² plating surface area to about 10,000 cells/mm² plating surface area.

7.-10. (canceled)

11. The method according to claim 1, comprising detaching the cells after incubating the cells in high cell density conditions.

12. The method according to claim 1, further comprising incubating differentiated cells in a medium comprising a modulator of 5′AMP-activated protein kinase or AMPK for a time and under conditions sufficient to produce a progenitor cell that is capable of being differentiated into a plurality of different cell types.

13. The method of claim 12, comprising incubating differentiated cells in a medium comprising a modulator of 5′AMP-activated protein kinase or AMPK for a period of time sufficient for phosphorylation and/or activation and/or stabilization of tumor suppressor p53 protein that delays or inhibits or represses cell cycle progression or cell division.

14. The method of claim 12, wherein the 5′ AMPK is selected from the group consisting of AICAR, a phosphorylated ZMP, Metformin, Compound C, thrombin, ghrelin, 3PG, extracellular AMP, a long chain fatty acyl analogs, acyl-CoA thioester, Dorsomorphin, glycogen, a PP ARa agonist (αA), a PPARα/γ dual agonist and phosphocreatine.

15. The method according to claim 1, further comprising incubating differentiated cells in a medium comprising a phorbol ester or active derivative thereof for a time and under conditions sufficient to produce a progenitor cell that is capable of being differentiated into a plurality of different cell types.

16. (canceled)

17. The method of claim 15, comprising incubating differentiated cells with an agent comprising as an active ingredient a phorbol ester derivative of formula (I): 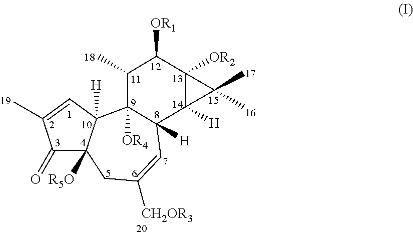
wherein R₁ is a hydrogen, or a butyryl, or a decanoyl, or a tetradecanoyl, or a N-methylaminobenzoyl group; R₂ is a formyl, or acetyl, or propionyl, or butyryl or pentanoyl, or hexanoyl, or benzoyl, or phenylacetyl group; R₃ is hydrogen or linoleic acid; R₄, and R₅ are each hydrogen.

18.-22. (canceled)

23. The method according to claim 1, further comprising incubating differentiated cells in a medium comprising a retinoid for a time and under conditions sufficient to produce a progenitor cell that is capable of being differentiated into a plurality of different cell types.

24. The method of claim 23, comprising incubating differentiated cells in a medium comprising an agonist and/or antagonist of a receptor or ligand of a retinoic acid or an isoform thereof.

25. The method of claim 23, wherein the retinoid is selected from the group consisting of ATRA, 9CRA, 13-cis retinoic acid, 11-cis retinoic acid, Am80, BMS189452, CD666, BMS188649, BMS185411, BMS188649, CD336/Am580, CD2019, CD437/AHPN, CD2665, CD2503, CD367, CD2314, CD 3640, AGN193109 and any combination thereof.

26. The method according to claim 23, comprising incubating differentiated cells in a medium comprising a retinoid capable of inducing trans-differentiation of the differentiated cells into the progenitor cells.

27. The method according to claim 23, comprising incubating differentiated cells in a medium comprising a retinoid at a final concentration of about 10⁻¹⁰ M to about 10⁻² M.

28.-34. (canceled)

35. The method according to claim 1, comprising detaching the cells by incubating the cells in a medium comprising EDTA, wherein said medium is substantially Ca²⁺-free and substantially Mg⁺-free so as to not interfere with detachment.

36. (canceled)

37. The method according to claim 1, comprising incubating the differentiated cells for a period of time sufficient to induce and/or increase expression of one or more gene products that delay or inhibit or repress cell cycle progression.

38. The method according to claim 37, wherein the one or more gene products that delay or inhibit or repress cell cycle progression are selected from p27^{Kip1}, p57^{Kip2} and p18.

39.-53. (canceled)

54. A method for producing and/or repairing and/or regenerating a tissue or an organ comprising incubating a progenitor cell, differentiated cell or cell culture, wherein the progenitor cell, differentiated cell or cell culture is a product of a method of claim 1 for a time and under conditions sufficient to produce and/or repair and/or regenerate one or more tissues or organs from the cell or cell culture, and culturing or perfusing the cells or cell culture onto or into a biocompatible scaffold or matrix for a time and under conditions sufficient for the cell or cell culture to produce and/or repair and/or regenerate one or more tissues or organs.

55. (canceled)

56. The method according to claim 54, wherein the scaffold or matrix comprises a decellularized tissue or organ or a derivative thereof.

57.-58. (canceled)

59. The method according to claim 54, further comprising providing the progenitor cells an agent selected from the group consisting of a neuropeptide Y (NPY), a fragment of neuropeptide Y, a variant of neuropeptide Y, a compound capable of inducing expression of a gene encoding a neuropeptide Y protein or fragment or variant thereof, a cell that produces a neuropeptide Y and an agonist or antagonist of a neuropeptide Y receptor, a neurotrophin, a fragment of a neurotrophin, a compound capable of inducing expression of a neurotrophin gene, and/or an agonist or antagonist of a receptor for a neurotrophin, a neuregulin, a fragment of a neuregulin, a compound capable of inducing expression of a neuregulin gene, and an agonist or antagonist of a receptor for neuregulin, and combinations thereof, wherein said agent induces regeneration, repair or building of a tissue or organ.

60.-62. (canceled)

63. A pharmaceutical composition comprising a progenitor cell, differentiated cell or cell culture, wherein the progenitor cell, differentiated cell or cell culture is a product of the method of claim 1 and a pharmaceutically acceptable carrier.

64.-70. (canceled)

71. A kit for regenerating and/or repairing and/or building a tissue or an organ, wherein said kit comprises:
(i) a progenitor cell, differentiated cell or cell culture, wherein the progenitor cell differentiated cell or a cell culture is a product of method claim 1;
(ii) a biocompatible scaffold or matrix;
(iii) optionally, at least one growth factor or mitogen or functional fragment thereof or nucleic acid encoding said growth factor, mitogen, morphogen or functional fragment thereof;
(iv) optionally, an agent selected from the group consisting of a neuropeptide Y (NPY), a fragment of neuropeptide Y, a variant of neuropeptide Y, a compound capable of inducing expression of a gene encoding a neuropeptide Y protein or fragment or variant thereof, a cell that produces a neuropeptide Y and an agonist or antagonist of a neuropeptide Y receptor, a neurotrophin, a fragment of a neurotrophin, a compound capable of inducing expression of a neurotrophin gene, and/or an agonist or antagonist of a receptor for a neurotrophin, a neuregulin, a fragment of a neuregulin, a compound capable of inducing expression of a neuregulin gene, and an agonist or antagonist of a receptor for neuregulin, and combinations thereof; and
(iv) optionally, directions for preparing, maintaining and/or using the cells or the scaffold material or matrix including any cell culture or tissue or organ derived therefrom.
